(19) United States
(12) Patent Application Publication
(10) Pub. No.: US 2010/0216813 A1
(43) Pub. Date: Aug. 26, 2010

(54) PYRROLIDINEANILINES

(75) Inventor: James S. Frazee, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: Smithkline Beecham Corporation

(21) Appl. No.: 12/095,505

(22) PCT Filed: Nov. 29, 2006
(86) PCT No.: PCT/US06/61317

§ 371 Date: May 30, 2008

Related U.S. Application Data
(60) Provisional application No. 60/740,965, filed on Nov. 30, 2005.

(51) Int. Cl. A61K 31/4025; C07D 207/14; C07D 405/10; C07D 409/06; C07D 401/06; C07D 403/06; A61K 31/40; A61K 31/4439; A61K 31/4178; A61K 31/454; A61K 31/506; A61K 31/497; A61P 15/00

(52) U.S. Cl. 514/255.05; 548/557; 548/526; 548/527; 546/279.1; 548/314.7; 548/542; 544/311; 544/336; 514/426; 514/422; 514/343; 514/397; 514/326; 514/274

(57) ABSTRACT

The present invention relates to a compound represented by the following formula I or a pharmaceutically acceptable salt thereof; wherein R¹, R², and y are defined herein. The present invention further relates to compositions that include the compound of the present invention as well as a method of treating a patient from endometreosis or uterine fibroids.

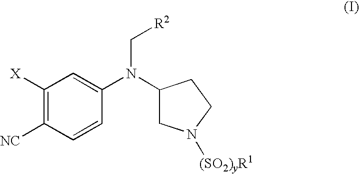

### BACKGROUND OF THE INVENTION

The present invention relates to a pyrrolidineaniline that is useful as a progesterone receptor modulator.

Endometriosis is a disease characterized by the growth of endometrial tissue (called lesions) at extrauterine sites. This lesion attachment can result in pain, dysmenorrhea, dyspareunia, and infertility. It is estimated that greater than 80% of patients presenting with chronic pelvic pain are eventually diagnosed with endometriosis. The prevalence of the disease is about 7-10% of women of reproductive years with a familial association risk increase of 10-fold. Definitive diagnosis is only reached by laparoscopy, but typically there is about a ten year delay from disease onset to conclusive diagnosis. Consistent with their uterine origins, it is believed that the endometriotic lesions are hormonally dependent upon estrogen; consequently, therapies that functionally antagonize estrogen production or action, such as drugs containing progesterone receptor (PR) modulators, are efficacious in alleviating symptoms. Current therapeutic goals include reducing pain with anti-inflammatory agents and suspending the ovarian cycle using hormonal modulation drugs.

Another disease believed to be hormonally responsive to estrogen is uterine leiomyomas (fibroids), which appear as benign uterine smooth muscle tumors occurring primarily in women of reproductive age. Fibroids occur at rates of 20-25% and are the leading indication for hysterectomies. The most common symptoms are menorrhagia, pelvic pain/discomfort, bladder and bowel compression symptoms, and possibly infertility. Medical treatments for leiomyomas consist of those commonly prescribed for endometriosis, with treatments containing progesterone receptor modulators being most common due to safety, tolerability, ease of use and cost.

Most drug development has focused on modulation by full agonism or antagonism of progesterone receptors. For example, progestins are molecules that interact with progesterone receptor to activate or repress gene expression in target cells in a manner presumed to be progesterone-like. Though progestins are used in oral contraception, hormone therapy, and treatment of reproductive disorders, such as endometriosis and leiomyomas, these agents cause a number of adverse effects, including breakthrough bleeding, mood altering, acne, weight gain, and breast tenderness. Paradoxically, progesterone receptor antagonists such as mifepristone have been suggested as potential therapies, but the data are limited with few patients and no placebo-controlled randomized trials.

D. DeManno et al. (Steroids 68 (2003) 1019-1032), report that asoprisnil is a progesterone receptor modulator with mixed agonist/antagonistic activities. While the efficacy of the agent in treatment of endometriosis or fibroids is uncertain, early data from healthy female subjects indicate that the agent induces endometrial atrophy and amenorrhea, which suggests a predominantly progesterone receptor antagonist action in humans. Unfortunately, PR antagonists such as RU-486 tend to be abortifacient.

Accordingly, it would be desirable to discover a way to suppress estrogen-dependent endometriotic growth while reducing the systemic effects associated with current progesterone receptor modulating therapy.

### SUMMARY OF THE INVENTION

In a first aspect, the present invention provides a compound represented by the following formula:

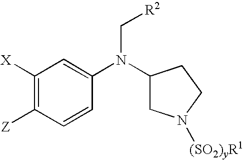

or a pharmaceutically acceptable salt thereof;
wherein Z is Cl, NO₂, OCH₃, or CN;

### X is H, F, Cl, Br, or CF₃;

R¹ is H, C₁-C₆-alkyl, CF₃, C₁-C₆-alkoxycarbonyl-C₁-C₆-alkyl, C₃-C₆-cycloalkyl, heterocycloalkyl, C₁-C₆-alkyl-heterocycloalkyl, heteroaryl-(R³)ₙ, phenyl-(R^{3′})ₙ, or —CH₂R⁴;
y is 0 or 1, with the proviso that when R¹ is H, y is 0; and
R² is C₁-C₅-alkyl, C₁-C₆-alkoxycarbonyl, C₁-C₆-alkoxycarbonyl-C₁-C₅-alkyl, C₁-C₆-alkyloxy-C₁-C₅-alkyl, C₃-C₆-cycloalkyl, heterocycloalkyl, C₁-C₆-alkyl-heterocycloalkyl, C₂-C₄-alkenyl, naphthyl, heteroaryl-(R³)ₙ, or phenyl-(R^{3′})ₙ, where n is 0, 1, 2, or 3;
each R³ is independently CH₃, F, Cl, Br, CF₃, C₁-C₆-alkoxy, C₁-C₆-alkoxycarbonyl, dimethylamino, C₂-C₄-alkenyl, or CN, or where 2 of the R³ groups, together with the heteroaryl ring to which they are attached form a fused bicyclic ring;
each R³ is independently C₁-C₆-alkyl, F, Cl, Br, CF₃, C₁-C₆-alkoxy, dimethylamino, amido, C₂-C₄-alkenyl, nitro, —COO—C₁-C₆-alkyl, OH, COOH, or CN, or where 2 of the R^{3′} groups, together with the phenyl ring to which they are attached form a fused bicyclic ring; and
R⁴ is F, Cl, Br, C₁-C₆-alkyloxy-C₁-C₆-alkyl, CF₃, CH₂CF₃, COOH, CH₂CN, CN, C₁-C₆-alkylcarbonyl, heterocycloalkyl, C₁-C₆-alkyl-heterocycloalkyl, heterocycloalkyl-CH₂—, aminocarbonyl, aminocarbonyl-CH₂—, di-C₁-C₆-alkylaminocarbonyl, C₂-C₄-alkenyl, hydroxy-C₁-C₆-alkyl, naphthyl, heteroaryl-(R³)ₙ, phenyl-(R^{3′})ₙ, or CH₂— phenyl-(R^{3′})ₙ.

Compounds of the present invention are useful as progesterone receptor modulators.

### DETAILED DESCRIPTION OF THE INVENTION

The present invention is a compound represented by the following formula:

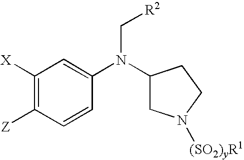

or a pharmaceutically acceptable salt thereof;
wherein Z is Cl, NO₂, OCH₃, or CN;

### X is H, F, Cl, Br, or CF₃;

R¹ is H, C₁-C₆-alkyl, CF₃, C₁-C₆-alkoxycarbonyl-C₁-C₆-alkyl, C₃-C₆-cycloalkyl, heterocycloalkyl, C₁-C₆-alkyl-heterocycloalkyl, heteroaryl-(R³)ₙ, phenyl-(R^{3′})ₙ, or —CH₂R⁴;
y is 0 or 1, with the proviso that when R¹ is H, y is 0; and
R² is C₁-C₅-alkyl, C₁-C₆-alkoxycarbonyl, C₁-C₆-alkoxycarbonyl-C₁-C₅-alkyl, C₁-C₆-alkyloxy-C₁-C₅-alkyl, C₃-C₆-cycloalkyl, heterocycloalkyl, C₁-C₆-alkyl-heterocycloalkyl, C₂-C₄-alkenyl, naphthyl, heteroaryl-(R³)ₙ, or phenyl-(R^{3′})ₙ, where n is 0, 1, 2, or 3;
each R³ is independently CH₃, F, Cl, Br, CF₃, C₁-C₆-alkoxy, C₁-C₆-alkoxycarbonyl, dimethylamino, C₂-C₄-alkenyl, or CN, or where 2 of the R³ groups, together with the heteroaryl ring to which they are attached form a fused bicyclic ring;
each R³ is independently C₁-C₆-alkyl, F, Cl, Br, CF₃, C₁-C₆-alkoxy, dimethylamino, amido, C₂-C₄-alkenyl, nitro, —COO—C₁-C₆-alkyl, OH, COOH, or CN, or where 2 of the R^{3′} groups, together with the phenyl ring to which they are attached form a fused bicyclic ring; and
R⁴ is F, Cl, Br, C₁-C₆-alkyloxy-C₁-C₆-alkyl, CF₃, CH₂CF₃, COOH, CH₂CN, CN, C₁-C₆-alkylcarbonyl, heterocycloalkyl, C₁-C₆-alkyl-heterocycloalkyl, heterocycloalkyl-CH₂—, aminocarbonyl, aminocarbonyl-CH₂—, di-C₁-C₆-alkylaminocarbonyl, C₂-C₄-alkenyl, hydroxy-C₁-C₆-alkyl, naphthyl, heteroaryl-(R³)ₙ, phenyl-(R^{3′})ₙ, or CH₂— phenyl-(R^{3′})ₙ.

As used herein, “C₁₋₆-alkyl” refers to a straight or branched chain monovalent radical of 1 to 6 carbon atoms, including, methyl, ethyl, n-propyl, isopropyl, n-butyl, isobutyl, t-butyl, n-pentyl, isopentyl, neopentyl, and n-hexyl and isomers thereof.

Examples of suitable C₁-C₆-alkoxy groups include methoxy and ethoxy groups; examples of suitable C₁-C₆-alkoxycarbonyl-C₁-C₆-alkyl groups include —C(CH₃)₂C(O)OCH₂CH₃ and —CH₂CH₂C(O)O— t-butyl groups; an example of a suitable C₁-C₆-alkoxy-C₁-C₆-alkyl group is CH₃OCH₂— (methoxymethyl); examples of suitable C₁-C₆-alkoxycarbonyl groups include —CO₂CH₂CH₃ and —CO₂-t-butyl groups; examples of suitable C₃-C₆-cycloalkyl groups include cyclopentyl and cyclohexyl groups.

As used herein, “heterocycloalkyl” refers to a 3-6-membered ring that contains at least one heteroatom selected from N, O, and S. Examples of suitable heterocycloalkyl groups include piperidinyl, pyrrolidinyl, pyrazinyl, morpholino, and 1,3-dioxolan-2-yl groups. Similarly, “C₁-C₆-alkyl-heterocycloalkyl” refers to a heterocycloalkyl group substituted with a C₁-C₆-alkyl group. An example of a C₁-C₆-alkyl-heterocycloalkyl group is N-methylpiperidinyl.

The term “heteroaryl” is used herein to describe an aromatic group that contains at least one heteroatom selected from N, O, and S. Examples of suitable heteroaryl groups include pyridinyl, oxidopyridinyl, furyl, thienyl, imidazolyl, pyrrolyl, pyrazolyl, oxazolyl, isoxazolyl, thiazolyl, isothiazolyl, triazolyl, tetrazolyl, pyrimidinyl, and benzothiadiazolyl groups. The heteroaryl and phenyl groups may also be substituted as described herein. Heteroaryl also includes more than one heteroaryl groups, for example, pyridinylthienyl and methoxypyridinylthienyl groups.

Examples of C₂₋₄-alkenyl groups include vinyl, allyl, and isopropenyl groups.

When R² is heteroaryl-(R³)ₙ or phenyl-(R^{3′})ₙ and n is 2 or 3, two of the R³ or R^{3′} groups can, together with the heteroaryl or phenyl groups respectively to which they are attached, form a fused bicyclic group. Examples of such fused bicyclic groups include benzodioxinyl and benzodioxolyl groups.

The term “IC₅₀” is used herein to refer to the molar concentration of a compound required to inhibit binding of 50% of Fluormone PL Red to the progesterone receptor. Furthermore, pIC₅₀ is the negative log of the molar IC₅₀.

The terms “a compound of the present invention” and “the compound of the present invention” are used herein to refer to one or more compounds of the present invention. The present invention includes compounds as well as their pharmaceutically acceptable salts alternatively or collectively. Accordingly, the word “or” in the context of a compound or a pharmaceutically acceptable salt thereof, is understood to include the alternative (either a compound or a pharmaceutically acceptable salt) as well as the collective (both the compound and its pharmaceutically acceptable salt).

Pharmaceutically acceptable salts of the compounds of the present invention include salts formed by the addition of an inorganic acid such as hydrochloric acid, hydrobromic acid, sulfuric acid, nitric acid, or phosphoric acid; or by the addition of an organic acid such as acetic acid, fumaric acid, succinic acid, maleic acid, citric acid, benzoic acid, p-toluenesulfonic acid, methanesulfonic acid, naphthalenesulfonic acid, or tartaric acid.

A skilled artisan will appreciate that the compound and/or pharmaceutically acceptable salt of the present invention includes all of its manifestations including amorphous and one or more crystalline forms or any combinations thereof. Crystalline forms include anhydrous forms as well as aqueous and non-aqueous solvate forms.

The compounds of the present invention may exist as optical isomers including diastereoisomers and enantiomers, and mixtures of isomers in all ratios including racemic mixtures. Indeed, another aspect of the present invention is a compound of the formula:

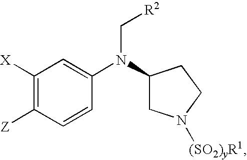

or a pharmaceutically acceptable salt thereof; where X, Z, R¹, R², and y are as previously defined.

The present invention also relates to a pharmaceutical composition comprising the compound of the formula of the present invention or a pharmaceutically-acceptable salt thereof; and a pharmaceutically acceptable earner therefor. The composition may be formulated for administration by any route, such as oral, topical or parenteral. The compositions may be in the form of tablets, capsules, powders, granules, lozenges, creams or liquid preparations, such as oral or sterile parenteral solutions or suspensions.

The present invention also relates to a method comprising administering to a patient in need thereof an effective amount of the compound of the formula of the present invention or a pharmaceutically-acceptable salt thereof to treat endometreosis or uterine fibroids. In addition the compound of the present invention can be combined with one or more exogenous estrogens prescribed for hormone therapy to reduce the risk of estrogen-dependent cancers such as endometrial cancer.

### Biological Assays

### Abbreviations

Acquest/Biosystems is a multi-mode reader (FP reader); CHAPS refers to 3-cholamidopropyl-dimethylammonio 1-propanesulfonate; DTT refers to dithiothreitol.

PR Binding Assay—The assay was performed according to the manufacturers protocol (PR Competitor Assay Kit, Red—(Invitrogen—Product No. P2962)) with minor amendments. Briefly, 40 nM PR-Ligand Binding Domain, 2 nM Fluormone PL Red and 1 mM DTT were dissolved and mixed in Complete PR RED Buffer supplemented with 2 mM CHAPS. 10 μL of the mix was dispensed to each well of Greiner low volume plates, containing compounds at the required concentration. The plates were spun for 1 min at 200 g, covered to protect the reagents from light, and then incubated at room temperature for approximately 2 hours. Plates were read on an Acquest using a 530-25 nm excitation and 580-10 nm emission interference filter and a 561 nm Dichroic mirror.

### Data Analysis

All data was normalized to the mean of 16 high and 16 low control wells on each plate. A four parameter curve fit of the following form was then applied

$$y = \frac{a - d}{1 + {( \frac{x}{c} )}^{b}} + d$$

Where a is the minimum, b is the Hill slope, c is the XC50 and d is the maximum. Data is presented as the mean pIC₅₀ with the standard deviation of the mean of n experiments.

### Methods of Use

The compounds of the present invention are useful as modulators of progesterone receptors and may be useful in the treatment of disease associated with endometreosis and uterine fibroids. Thus, the present invention further relates to a method of treating a patient comprising administering to the patient an effective amount of a compound of formula I or a pharmaceutically-acceptable salt thereof or combination thereof to treat endometreosis or uterine fibroids.

### Synthetic Schemes

Compounds of the present invention can be prepared, for example, in accordance with the following schemes. As used herein, Boc refers to t-butoxycarbonyl; DMSO refers to dimethyl sulfoxide; DMF refers to dimethylformamide; TFA refers to trifluoroacetic acid; DCE refers to dichloroethane; and Et₂O refers to diethyl ether.

In a first step, illustrated in Scheme 1, nucleophilic addition of pyrrolidine 1a to a phenyl fluoride forms aniline 1b or 1f. In the case where X=Cl, aniline 1b can be hydrogenated to form aniline 1c. Alternatively, reductive amination of aniline 1 h with ketone 1i yields secondary aniline 1j. Compounds 1b, 1c, 1f, or 1j can be further modified by treatment with strong base and electrophile to form the respective tertiary anilines and subsequent removal of the Boc protecting group under acidic conditions forms compounds 1e, 1g, and 1k.

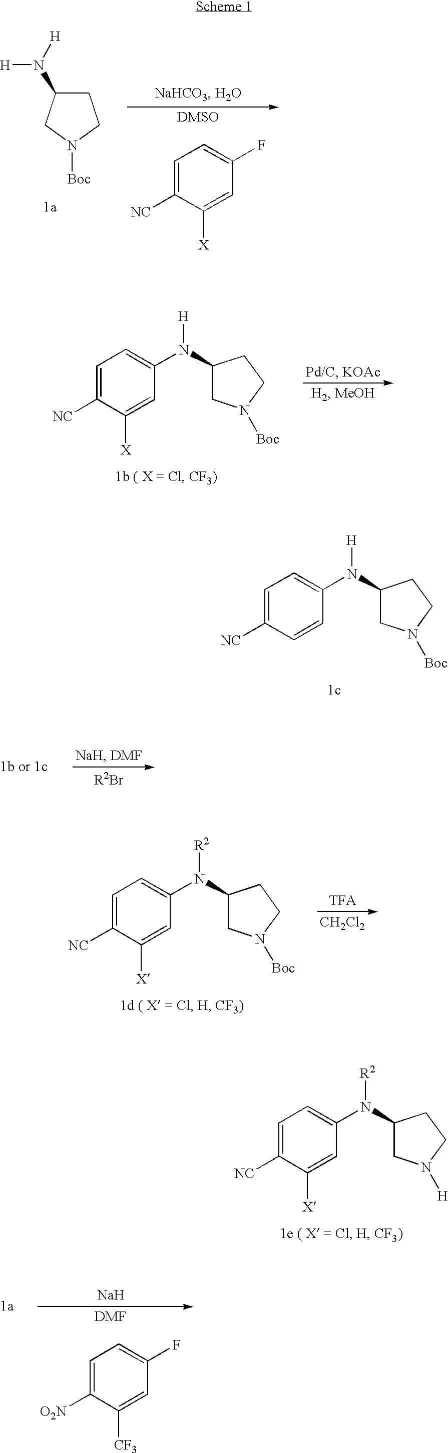

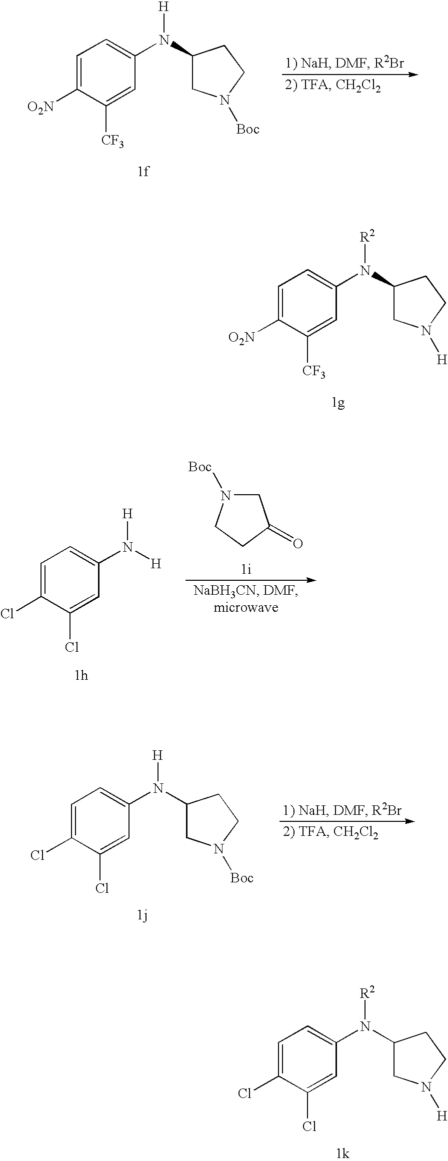

Compounds 1e, 1g, and 1k can be further functionalized in accordance with Scheme 2:

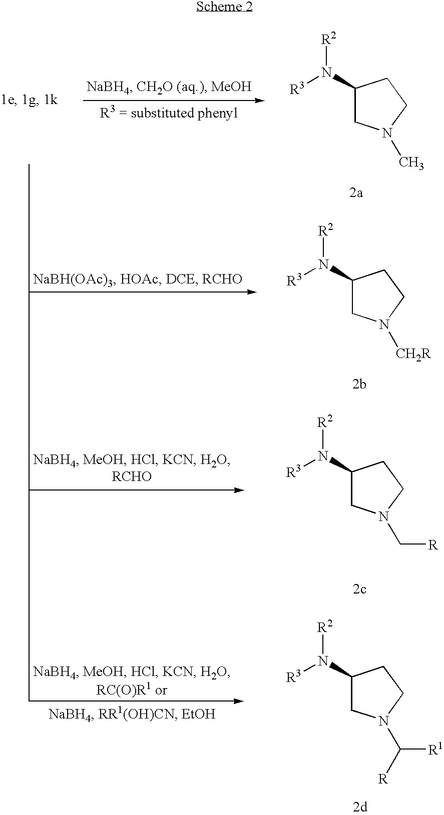

Treatment of the pyrrolidine 1e with alkyl or benzyl bromides or t-butyl acrylate (Scheme 3) under basic conditions can afford the desired pyrrolidines 3a and 3b (Method E or F) while reaction of 1e with McSO₂Cl and pyridine yields the desired sulfonamide 3c (Method G).

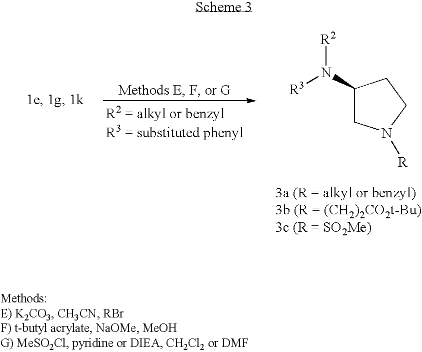

Pyrrolidine 1b can be deprotected under acidic conditions to form 4a and the pyrrolidine nitrogen can be selectively functionalized to form 4b-4e (Scheme 4).

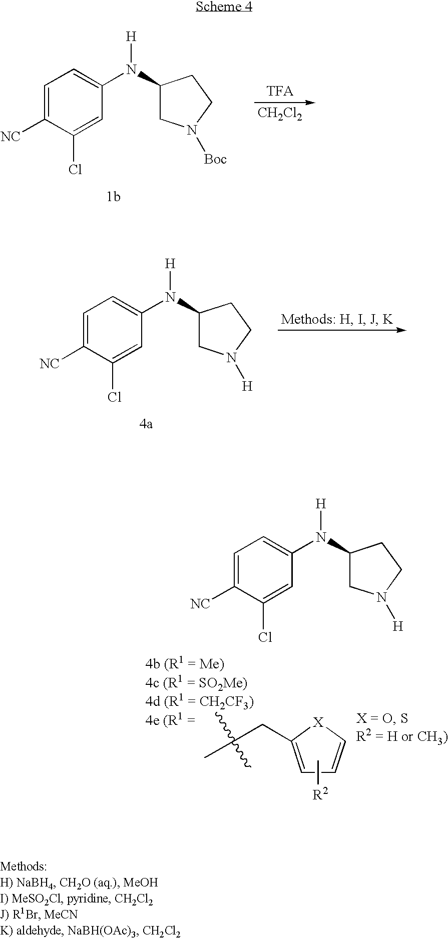

The aniline nitrogen can then be modified, for example, as shown in Scheme 5.

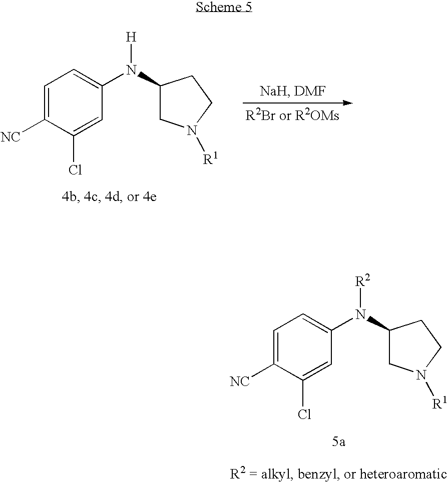

Scheme 6 illustrates the synthesis of various electrophiles that can be used to make compounds of the present invention:

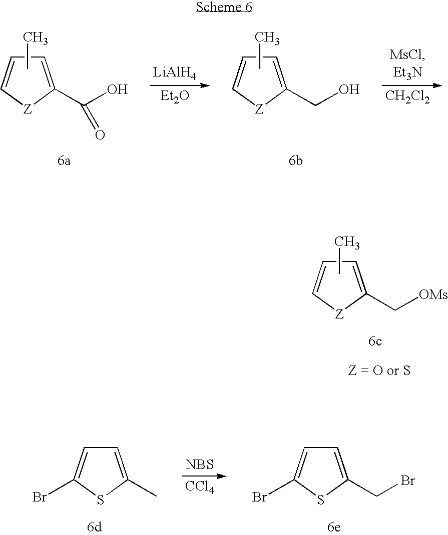

It may be desirable to further derivatize a compound of the present invention. For example, thiophene bromide 7a can undergo a Suzuki reaction to yield 7b or the methyl ester 7c can be hydrolyzed to the acid 7d (Scheme 7). Moreover, as illustrated in Scheme 8, the ethyl ester 8a can be hydrolyzed under basic conditions to the acid 8b, which then can be reacted with LiAlH₄ to yield the primary alcohol 8c.

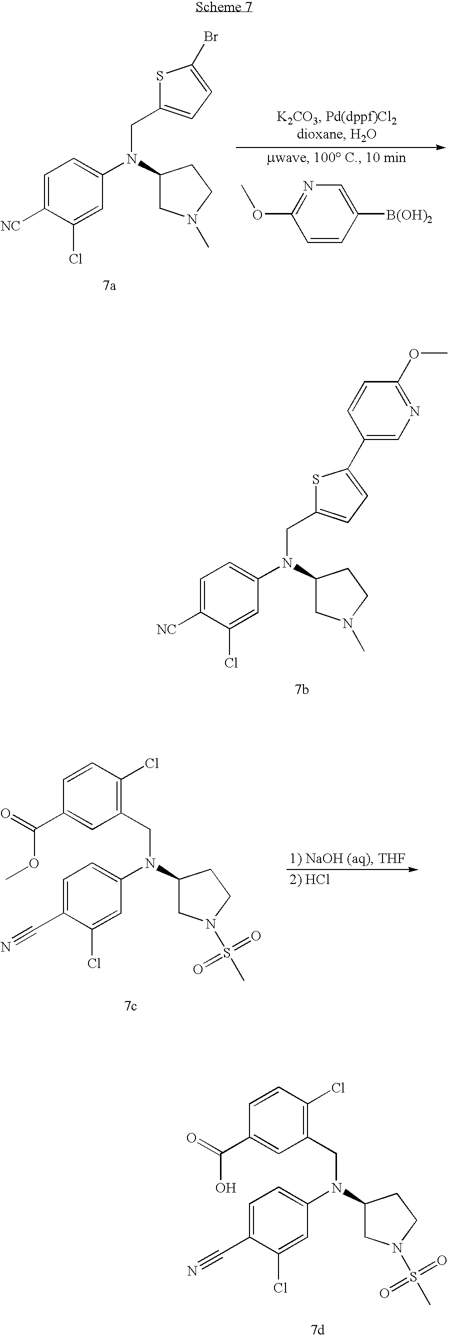

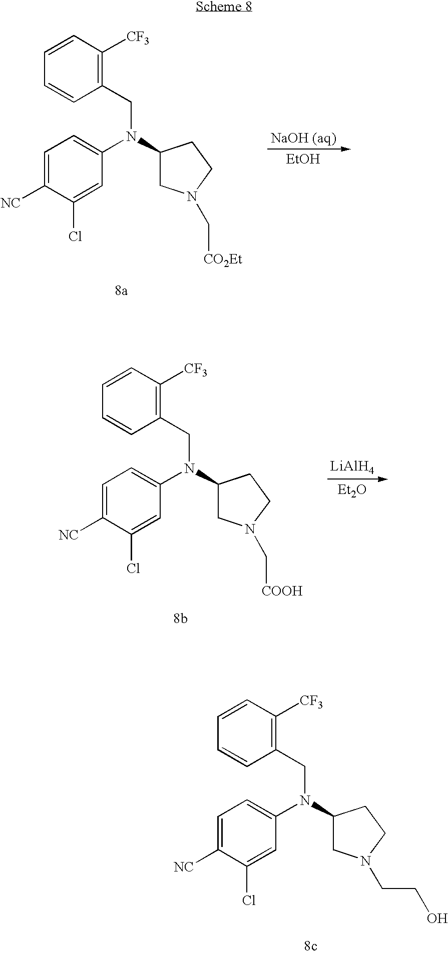

Treatment of pyrrolidine 9a with PtO₂ and H₂ forms the desired compound 9b (Scheme 9). Moreover, as shown in Scheme 10, reaction of 10a or 10b under similar conditions yields the desired functionalized pyrrolidines 10c or 10d, respectively.

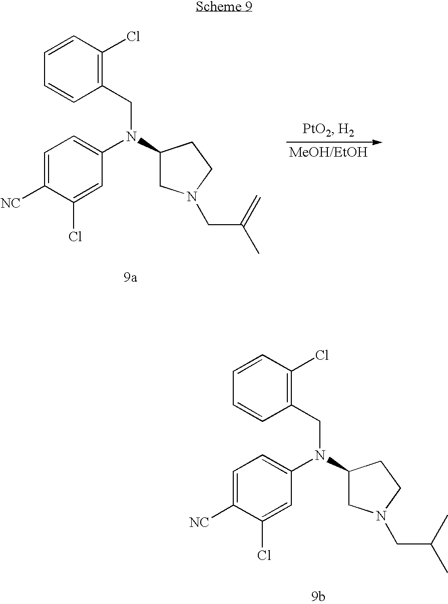

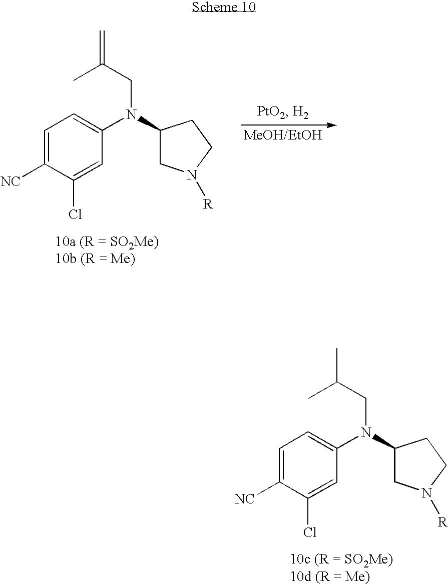

### Examples

The following Examples are for illustrative purposes only and are not intended to limit the scope of the invention. The compounds from these Examples exhibit a pIC₅₀ of greater than 5 (i.e., an IC₅₀ of less than 10 μM). The phrase “IC₅₀ of less than 10 μM” refers to an IC₅₀ of less than 10 μM as measured using the PR binding assay described herein.

### Abbreviations

RT=room temperature

### THF=Tetrahydrofuran

EtOAc=Ethyl acetate
Et₂O=Diethyl ether
DMSO=Dimethyl sulfoxide

### DMF=N,N-Dimethylformamide

### MeOH=Methanol

### EtOH=Ethanol

TFA=Trifluoroacctic acid

### MeCN=Acetonitrile

NaBH(OAc)₃=Sodium triacetoxyborohydride

### TEA=Triethylamine

KOAc=Potassium acetate
AcOH=Acetic acid
KCN=Potassium cyanide
NaOMe=Sodium methoxide

### NBS=N-Bromosuccinimide

AIBN=2,2′-Azobis(2-methylpropionitrile)

### DIEA=N,N-Diisopropylethylamine

Pd(dppf)Cl₂=[1,1′-Bis(diphenylphosphino)ferrocene]dichloropalladium(II) complex

### General

Proton nuclear magnetic resonance (¹H NMR) spectra were recorded at 400 MHz, and chemical shifts are reported in parts per million (ppm) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform, DMSO-d₆ is hexadeuteriodimethylsulfoxide, and CD₃OD or d₄-CH₃OH is tetradeuteriomethanol. Mass spectra were obtained using electrospray (ES) or atmospheric pressure chemical ionization (APCI) techniques. E. Merck Silica Gel 60 F-254 thin layer plates were used for thin layer chromatography. Flash chromatography was carried out on E. Merck Kieselgel 60 (230-400 mesh) silica gel or on an ISCO Combi-flash purification system using pre-filled silica gel cartridges. Preparative HPLC was performed using Gilson chromatography systems using a 30×100 mm Xterra Prep RP column at a flow rate of 40 mL/min. The solvent system used was a variable gradient of 18% to 90% acetonitrile/water using either 0.1% TFA or ammonium hydroxide to adjust the pH to 10. Celite® is a filter aid composed of acid-washed diatomaceous silica, and is a registered trademark of Manville Corp., Denver, Colo.

### Example 1

### 2-chloro-4-((3S)-3-pyrrolidinyl{[2-(trifluoromethyl)phenyl]methyl}amino)benzonitrile

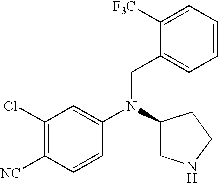

### a) 1,1-dimethylethyl (3S)-3-[(3-chloro-4-cyanophenyl)amino]-1-pyrrolidinecarboxylate

A mixture of 2-chloro-4-fluorobenzonitrile (4.26 g, 27.5 mmol), 1,1-dimethylethyl (3S)-3-amino-1-pyrrolidinecarboxylate (5.11 g, 27.5 mmol) and NaHCO₃ (4.62 g, 55 mmol) in 45 mL of DMSO and 5 mL of H₂O was heated with stirring at 96° C. for 6 h and 86° C. for 16 h. The reaction was diluted with 200 mL of H₂O and extracted with Et₂O (3×). The extracts were washed with H₂O (2×), dried over Na₂SO₄, filtered, and concentrated. The residue was crystallized from Et₂O hexane to yield the titled compound (6.88 g, 78%). LC-MS (ES) m/e 322 [M+H]⁺.

### b) 1,1-dimethylethyl (3S)-3-((3-chloro-4-cyanophenyl){[2-(trifluoromethyl)phenyl]methyl}amino)-1-pyrrolidinecarboxylate

NaH (60% dispersion in mineral oil, 1.95 g, 48.7 mmol) was washed free of mineral oil with hexane, suspended in 100 mL of DMF stirred, and cooled in an ice bath. A solution of 1,1-dimethylethyl (3S)-3-[(3-chloro-4-cyanophenyl)amino]-1-pyrrolidinecarboxylate (10.43 g, 32.5 mmol) in 40 mL of DMF was added dropwise over 20 min. The reaction was stirred an additional 40 min, and a solution of 1-(bromomethyl)-2-(trifluoromethyl)benzene (11.65 g, 48.7 mmol) in 25 mL of DMF was rapidly added. The reaction mixture was stirred for 30 min at 0° C. and 30 min at RT. The mixture was poured into 200 mL of cold aqueous NH₄Cl, and extracted with Et₂O (3×). The extracts were washed with H₂O (2×), dried over NaSO₄, filtered, and concentrated. The residue was purified by column chromatography (eluted with 25% EtOAc/hexane) to yield the titled compound (14.28 g, 92%). LC-MS (ES) m/e 480 [M+H]⁺.

### c) 2-chloro-4-((3S)-3-pyrrolidinyl{[2-(trifluoromethyl)phenyl]methyl}amino)benzonitrile

A solution of 1,1-dimethylethyl (3S)-3-((3-chloro-4-cyanophenyl){[2-(trifluoromethyl)phenyl]methyl}amino)-1-pyrrolidinecarboxylate (14.0 g, 29 mmol) in 16 mL of CH₂Cl₂ was treated with TFA (15 mL) and stirred for 1.5 h. The reaction mixture was concentrated. The residue was triturated with Et₂O and 0.1 mL of MeOH to yield the trifluoroacetate salt as a white solid. The heterogeneous mixture was filtered, washed with Et₂O, and dried. The trifluoroacetate salt was dissolved in 20 mL of MeOH and added to a stirred mixture of aqueous K₂CO₃ and Et₂O. The Et₂O was separated and the aqueous phase was extracted with Et₂O (2×). The combined Et₂O extracts were washed with H₂O and saturated NaCl, dried over Na₂SO₄, filtered, and concentrated to yield the titled compound (10.74 g, 98%). LC-MS (ES) m/e 380 [M+H]⁺.

### Example 2

### 2-chloro-4-{[(2-chlorophenyl)methyl][(3S)-3-pyrrolidinyl]amino}benzonitrile

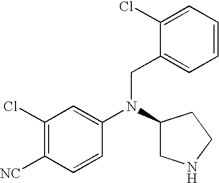

This compound was made according to general procedures of example 1 (part b and c) from 1-(bromomethyl)-2-chlorobenzene. LC-MS (ES) m/e 346 [M+H]⁺.

### Example 3

### 2-chloro-4-{(phenylmethyl)[(3S)-3-pyrrolidinyl]amino}benzonitrile

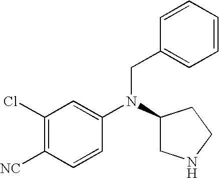

This compound was made according to general procedures of example 1 (part b and c) from benzyl bromide. LC-MS (ES) m/e 312 [M+H]⁺.

### Example 4

### 2-chloro-4-{[(2-methylphenyl)methyl][(3S)-3-pyrrolidinyl]amino}benzonitrile

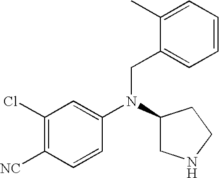

This compound was made according to general procedures of example 1 (part b and c) from 1-(bromomethyl)-2-methylbenzene. LC-MS (ES) m/e 326 [M+H]⁺.

### Example 5

### 2-chloro-4-{[(2-fluorophenyl)methyl][(3S)-3-pyrrolidinyl]amino}benzonitrile

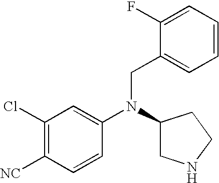

This compound was made according to general procedures of example 1 (part b and c) from 1-(bromomethyl)-2-fluorobenzene. LC-MS (ES) m/e 330 [M+H]⁺.

### Example 6

### 4-((3S)-3-pyrrolidinyl{[2-(trifluoromethyl)phenyl]methyl}amino)-2-(trifluoromethyl)benzonitrile

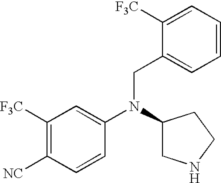

### a) 1,1-dimethylethyl (3S)-3-[(3-trifluoromethyl-4-cyanophenyl)amino]-1-pyrrolidinecarboxylate

This compound was made according to general procedure of example 1 (part a) from 4-fluoro-2-trifluoromethylbenzonitrile. LC-MS (ES) m/e 356 [M+H]⁺.

### b) 1,1-dimethylethyl (3S)-3-([4-cyano-3-(trifluoromethyl)phenyl]{[2-(trifluoromethyl)phenyl]methyl}amino)-1-pyrrolidinecarboxylate

This compound was made according to general procedure of example 1 (part b) from 1,1-dimethylethyl (3S)-3-[(3-trifluoromethyl-4-cyanophenyl)amino]-1-pyrrolidinecarboxylate. LC-MS (ES) m/e 514 [M+H]⁺.

### c) 4-((3S)-3-pyrrolidinyl{[2-(trifluoromethyl)phenyl]methyl}amino)-2-(trifluoromethyl)benzonitrile

This compound was made according to general procedure of example 1 (part c) from 1,1-dimethylethyl (3S)-3-([4-cyano-3-(trifluoromethyl)phenyl]{[2-(trifluoromethyl)phenyl]methyl}amino)-1-pyrrolidinecarboxylate LC-MS (ES) m/e 414 [M+H]⁺.

### Example 7

### 4-{[(2-chlorophenyl)methyl][(3S)-3-pyrrolidinyl]amino}-2-(trifluoromethyl)benzonitrile

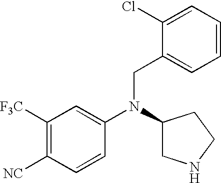

This compound was made according to general procedure of example 1 (part b) from 1-(bromomethyl)-2-chlorobenzene and 1,1-dimethylethyl (3S)-3-([4-cyano-3-(trifluoromethyl)phenyl]{[2-(trifluoromethyl)phenyl]methyl}amino)-1-pyrrolidinecarboxylate.

LC-MS (ES) m/e 380 [M+H]⁺.

### Example 8

### 4-((3S)-3-pyrrolidinyl{[2-(trifluoromethyl)phenyl]methyl}amino)benzonitrile

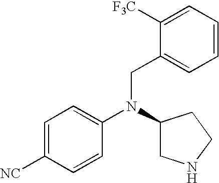

### a) 1,1-dimethylethyl (3S)-3-[(4-cyanophenyl)amino]-1-pyrrolidinecarboxylate

A solution of 1,1-dimethylethyl (3S)-3-[(3-chloro-4-cyanophenyl)amino]-1-pyrrolidinecarboxylate (400 mg, 1.25 mmol) and KOAc (400 mg) in 25 mL of MeOH was treated with 5% Pd/C (40 mg) and the mixture was hydrogenated at 1 atmosphere H₂ pressure for 25 min. The reaction mixture was filtered through Celite (to remove the catalyst) and the filtrate was concentrated. The residue was dissolved in Et₂O, washed with H₂O, dried over Na₂SO₄, filtered, and concentrated. The residue was crystallized from a mixture of CH₂Cl₂ and hexane to yield the titled compound (320 mg, 89%). LC-MS (ES) m/e 288 [M+H]⁺.

### b) 1,1-dimethylethyl (3S)-3-([4-cyanophenyl]{[2-(trifluoromethyl)phenyl]methyl}amino)-1-pyrrolidinecarboxylate

This compound was made according to general procedure of example 1 (part b) from 1,1-dimethylethyl (3S)-3-[4-cyanophenyl)amino]-1-pyrrolidinecarboxylate and 2-trifluoromethylbenzyl bromide. LC-MS (ES) m/e 446 [M+H]⁺.

### c) 4-((3S)-3-pyrrolidinyl{[2-(trifluoromethyl)phenyl]methyl}amino)benzonitrile

This compound was made according to general procedure of example 1 (part c) from 1,1-dimethylethyl (3S)-3-((4-cyanophenyl){[2-(trifluoromethyl)phenyl]methyl}amino)-1-pyrrolidinecarboxylate. LC-MS (ES) m/c 346 [M+H]⁺.

### Example 9

### 2-chloro-4-([(3S)-1-methyl-3-pyrrolidinyl]{[2-(trifluoromethyl)phenyl]methyl}amino)benzonitrile

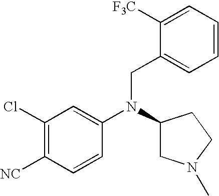

A solution of 2-chloro-4-((3S)-3-pyrrolidinyl{[2-(trifluoromethyl)phenyl]methyl}amino)benzonitrile (9.0 g, 24 mmol) in 125 mL of McOH was treated with formaldehyde (10.9 mL of a 37% aqueous solution, 142 mmol). After 30 min, the solution was cooled in an ice bath. NaBH₄ (2.0 g, 52 mmol) was slowly added and the reaction stirred for 30 min. Successively added were 200 mL of cold H₂O and aqueous NH₄Cl until the excess NaBH₄ was decomposed. The mixture was extracted with Et₂O. The combined extracts were washed with H₂O and concentrated. The residue was purified on a short Al₂O₃ (neutral, Brockman 2.8) column (eluted with Et₂O) to yield the titled compound (8.31 g, 88%). LC-MS (ES) m/e 394 [M+H]⁺.

### Example 10

### 2-chloro-4-{[(2-chlorophenyl)methyl][(3S)-1-methyl-3-pyrrolidinyl]amino}benzonitrile

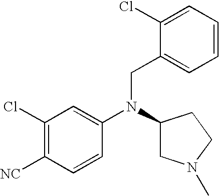

This compound was made according to general procedure of example 9 from 2-chloro-4-{[(2-chlorophenyl)methyl][(3S)-3-pyrrolidinyl]amino}benzonitrile. LC-MS (ES) m/e 360 [M+H]⁺.

### Example 11

### 4-([(3S)-1-methyl-3-pyrrolidinyl]{[2-(trifluoromethyl)phenyl]methyl}amino)-2-(trifluoromethyl)benzonitrile

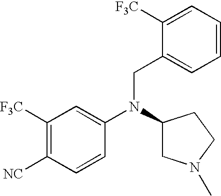

This compound was made according to general procedure of example 9 from 4-((3S)-3-pyrrolidinyl{[2-(trifluoromethyl)phenyl]methyl}amino)-2-(trifluoromethyl)benzonitrile. LC-MS (ES) m/e 428 [M+H]⁺.

### Example 12

### 4-{[(2-chlorophenyl)methyl][(3S)-1-methyl-3-pyrrolidinyl]amino}-2-(trifluoromethyl)benzonitrile

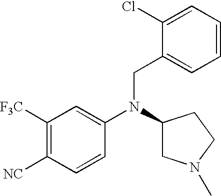

This compound was made according to general procedure of example 9 from 4-{[(2-chlorophenyl)methyl][(3S)-3-pyrrolidinyl]amino}-2-(trifluoromethyl)benzonitrile. LC-MS (ES) m/e 394 [M+H]⁺.

### Example 13

### 2-chloro-4-[[(3S)-1-methyl-3-pyrrolidinyl](phenylmethyl)amino]benzonitrile

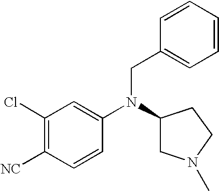

This compound was made according to general procedures of example 9 from 2-chloro-4-{(phenylmethyl)[(3S)-3-pyrrolidinyl]amino}benzonitrile. LC-MS (ES) m/e 326 [M+H]⁺.

### Example 14

### 2-chloro-4-{[2-methylphenyl)methyl][(3S)-1-methyl-3-pyrrolidinyl]-amino}benzonitrile

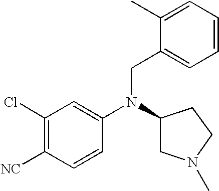

This compound was made according to general procedures of example 9 from 2-chloro-4-{[(2-methylphenyl)methyl][(3S)-3-pyrrolidinyl]amino}benzonitrile. LC-MS (ES) m/e 340 [M+H]⁺.

### Example 15

### 2-chloro-4-{[2-fluorophenyl)methyl][(3S)-1-methyl-3-pyrrolidinyl]amino}benzonitrile

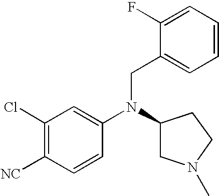

This compound was made according to general procedures of example 9 from 2-chloro-4-{[(2-fluorophenyl)methyl][(3S)-3-pyrrolidinyl]amino}benzonitrile. LC-MS (ES) m/e 344 [M+H]⁺.

### Example 16

### 4-([(3S)-1-methyl-3-pyrrolidinyl]{[2-(trifluoromethyl)phenyl]methyl}amino)benzonitrile

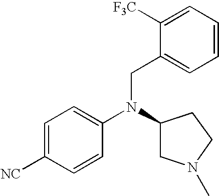

This compound was made according to general procedures of example 9 from 4-((3S)-3-pyrrolidinyl{[2-(trifluoromethyl)phenyl]methyl}amino)benzonitrile. LC-MS (ES) m/e 360 [M+H]⁺.

### Example 17

### 2-chloro-4-({(3S)-1-[2-cyanophenyl)methyl]-3-pyrrolidinyl}{[2(trifluoromethyl)phenyl]methyl}amino)benzonitrile

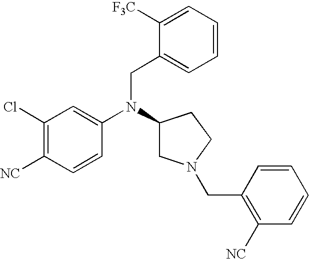

A solution of 2-chloro-4-((3S)-3-pyrrolidinyl{[2-(trifluoromethyl)phenyl]methyl}amino)benzonitrile (169 mg, 0.45 mmol) and 2-cyanobenzaldehyde (59 mg, 0.45 mmol) in 10 mL of 1,2-dichloroethane and 1 drop of AcOH was treated with NaBH(OAc)₃ (143 mg, 0.68 mmol), and the reaction was stirred 2 h. The reaction mixture was concentrated and the residue was dissolved in a mixture of H₂O and Et₂O. The organic solution was separated, washed with H₂O, dried over Na₂SO₄, filtered, and concentrated. The residue was purified by column chromatography on an Al₂O₃ column (neutral, Brockman 2.8) (eluted with Et₂O) to yield the titled compound (196 mg, 88%). LC-MS (ES) m/e 495 [M+H]⁺.

### Example 18

### 2-chloro-4-({(3S)-1-[(4-fluorophenyl)methyl]-3-pyrrolidinyl}{[2-(trifluoromethyl)phenyl]methyl}amino)benzonitrile

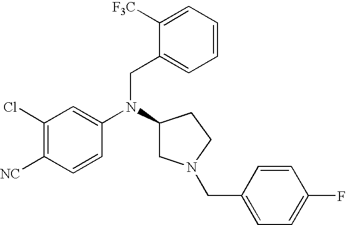

This compound was made according to general procedure of example 17 from 4-fluorobenzaldehyde. LC-MS (ES) m/e 488 [M+H]⁺.

### Example 19

### 2-chloro-4-({[2-(trifluoromethyl)phenyl]methyl}{(3S)-1-[(2,3,5-trifluorophenyl)methyl]-3-pyrrolidinyl}amino)benzonitrile

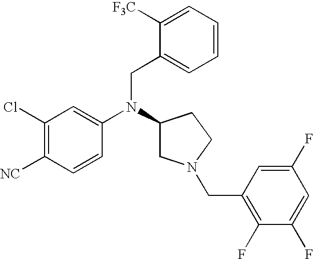

This compound was made according to general procedure of example 17 from 2,3,5-trifluorobenzaldehyde. LC-MS (ES) m/e 524 [M+H]⁺.

### Example 20

### 2-chloro-4-({(3S)-1-[(2,5-difluorophenyl)methyl]-3-pyrrolidinyl}{[2-(trifluoromethyl)phenyl]methyl}amino)benzonitrile

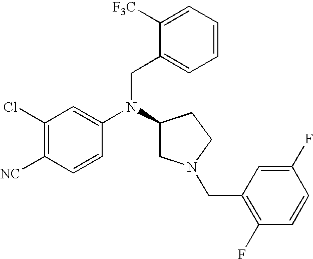

This compound was made according to general procedure of example 17 from 2,5-difluorobenzaldehyde. LC-MS (ES) m/e 506 [M+H]⁺.

### Example 21

### 2-chloro-4-({(3S)-1-[(1-oxido-4-pyridinyl)methyl]-3-pyrrolidinyl}{[2-(trifluoromethyl)phenyl]methyl}amino)benzonitrile

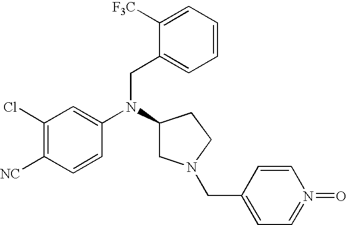

This compound was made according to general procedure of example 17 from pyridine-4-carboxaldehyde-N-oxide. LC-MS (ES) m/e 487 [M+H]⁺.

### Example 22

### 2-chloro-4-([(3S)-1-(2,3-dihydro-1,4-benzodioxin-6-ylmethyl)-3-pyrrolidinyl]{[2-(trifluoromethyl)phenyl]methyl}amino)benzonitrile

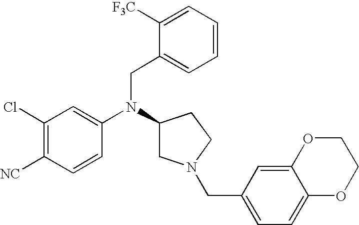

This compound was made according to general procedure of example 17 from 2,3-dihydro-1,4-benzodioxin-6-carboxaldehyde. LC-MS (ES) m/e 528 [M+H]⁺.

### Example 23

### 2-chloro-4-([(2-methylphenyl)methyl]{(3S)-1-[(5-methyl-2-thienyl)methyl]pyrrolidinyl}amino)benzonitrile

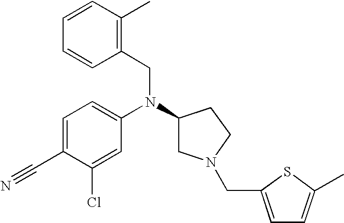

This compound was made according to general procedure of example 17 from 2-chloro-4-{[(2-methylphenyl)methyl][(3S)-3-pyrrolidinyl]amino}benzonitrile and 5-methyl-2-thiophenecarbaldehyde. LC-MS (ES) m/e 436.2 [M+H]⁺.

### Example 24

### 2-chloro-4-{[(2-methylphenyl)methyl][(3S)-1-(2-thienylmethyl)-3-pyrrolidinyl]amino}benzonitrile

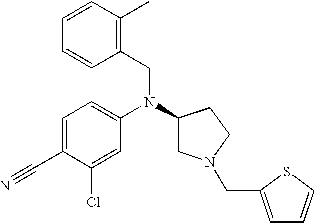

This compound was made according to general procedure of example 17 from 2-chloro-4-{[(2-methylphenyl)methyl][(3S)-3-pyrrolidinyl]amino}benzonitrile and 2-thiophenecarbaldehyde. LC-MS (ES) m/e 422.2 [M+H]⁺.

### Example 25

### 2-chloro-4-([(2-methylphenyl)methyl]{(3S)-1-[(3-methyl-2-thienyl)methyl]-3-pyrrolidinyl}amino)benzonitrile

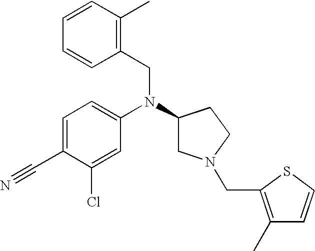

This compound was made according to general procedure of example 17 from 2-chloro-4-{[(2-methylphenyl)methyl][(3S)-3-pyrrolidinyl]amino}benzonitrile and 3-methyl-2-thiophenecarbaldehyde. LC-MS (ES) m/e 436.2 [M+H]⁺.

### Example 26

### 2-chloro-4-{[(2-methylphenyl)methyl][(3S)-1-(4-pyridinylmethyl)-3-pyrrolidinyl]amino}benzonitrile

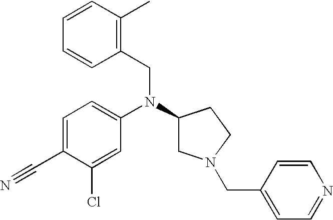

This compound was made according to general procedure of example 17 from 2-chloro-4-{[(2-methylphenyl)methyl][(3S)-3-pyrrolidinyl]amino}benzonitrile and 4-pyridinecarbaldehyde. LC-MS (ES) m/e 417.6 [M+H]⁺.

### Example 27

### 2-chloro-4-{[(3S)-1-(2-furanylmethyl)-3-pyrrolidinyl][(2-methylphenyl)methyl]amino}benzonitrile

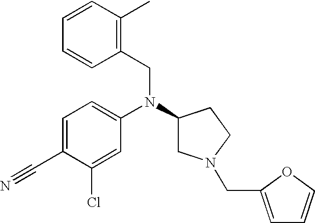

This compound was made according to general procedure of example 17 from 2-chloro-4-{[(2-methylphenyl)methyl][(3S)-3-pyrrolidinyl]amino}benzonitrile and 2-furancarbaldehyde. LC-MS (ES) m/e 406.4 [M+H]⁺.

### Example 28

### 2-chloro-4-{{(3S)-1-[(5-methyl-2-furanyl)methyl]-3-pyrrolidinyl}[2-methylphenyl)methyl]amino}benzonitrile

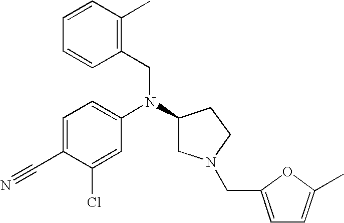

This compound was made according to general procedure of example 17 from 2-chloro-4-{[(2-methylphenyl)methyl][(3S)-3-pyrrolidinyl]amino}benzonitrile and 5-methyl-2-furancarbaldehyde.

LC-MS (ES) m/e 420.4 [M+H]⁺.

### Example 29

### 2-chloro-4-{((3S)-1-{[4-(methyloxy)phenyl]methyl}-3-pyrrolidinyl)[(2-methylphenyl)methyl]amino}benzonitrile

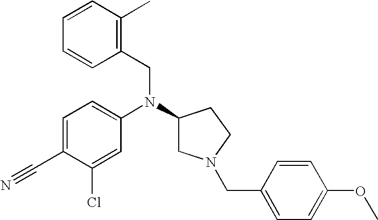

This compound was made according to general procedure of example 17 from 2-chloro-4-{[(2-methylphenyl)methyl][(3S)-3-pyrrolidinyl]amino}benzonitrile and 4-(methyloxy)benzaldehyde.

LC-MS (ES) m/e 445.8 [M+H]⁺.

### Example 30

### 2-chloro-4-([(2-chlorophenyl)methyl]{(3S)-1-[(1-methyl-1H-imidazol-2-yl)methyl]-3-pyrrolidinyl}amino)benzonitrile

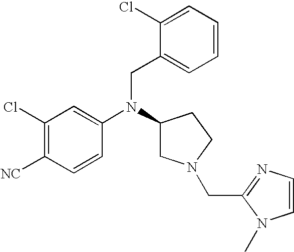

A solution of 2-chloro-4-{[(2-chlorophenyl)methyl][(3S)-3-pyrrolidinyl]amino}benzonitrile (180 mg, 0.52 mmol) and 1-methylimidazole-2-carboxaldehyde (58 mg, 0.52 mmol) in 5 mL of EtOH was treated with a solution of KCN (34 mg, 0.52 mmol) in 0.3 mL of H₂O and 1.0 N HCl (0.52 mL), and stirred for 16 h. NaBH₄ (200 mg) was added and the reaction heated to 50° C. for 15 min. The reaction was diluted with H₂O and extracted with Et₂O. The extracts were washed with H₂O, dried over Na₂SO₄, and concentrated to yield the titled compound (225 mg, 100%). LC-MS (ES) m/e 440 [M+H]⁺.

### Example 31

### 2-chloro-4-([(3S)-1-(1H-imidazol-2-ylmethyl)-3-pyrrolidinyl]{[2-(trifluoromethyl)phenyl]methyl}amino)benzonitrile

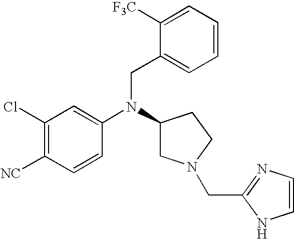

This compound was made according to general procedure of example 30 from 2-chloro-4-((3S)-3-pyrrolidinyl{[2-(trifluoromethyl)phenyl]methyl}amino)benzonitrile and imidazole-2-carboxaldehyde. LC-MS (ES) m/c 460 [M+H]⁺.

### Example 32

### 2-chloro-4-({(3S)-1-[(2-fluorophenyl)methyl]-3-pyrrolidinyl}{[2-(trifluoromethyl)phenyl]methyl}amino)benzonitrile

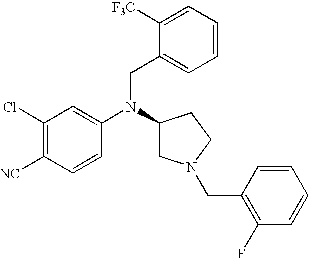

This compound was made according to general procedure of example 30 from 2-chloro-4-((3S)-3-pyrrolidinyl{[2-(trifluoromethyl)phenyl]methyl}amino)benzonitrile and 2-fluorobenzaldehyde.

LC-MS (ES) m/e 488 [M+H]⁺.

### Example 33

### 2-chloro-4-(((3S)-1-{[4-(methyloxy)phenyl]methyl}-3-pyrrolidinyl){[2-(trifluoromethyl)phenyl]methyl}amino)benzonitrile

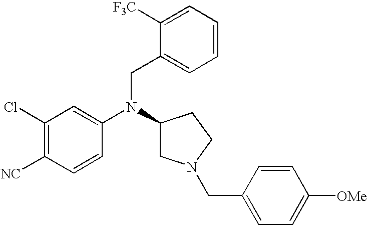

This compound was made according to general procedure of example 30 from 2-chloro-4-((3S)-3-pyrrolidinyl{[2-(trifluoromethyl)phenyl]methyl}amino)benzonitrile and p-anisaldehyde. LC-MS (ES) m/e 500 [M+H]⁺.

### Example 34

### 2-chloro-4-(((3S)-1-{[4-(dimethylamino)phenyl]methyl}-3-pyrrolidinyl) {[2-(trifluoromethyl)phenyl]methyl}amino)benzonitrile

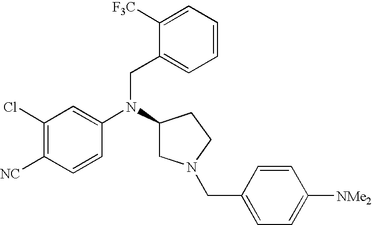

This compound was made according to general procedure of example 30 from 2-chloro-4-((3S)-3-pyrrolidinyl{[2-(trifluoromethyl)phenyl]methyl}amino)benzonitrile and p-dimethylaminobenzaldehyde. LC-MS (ES) m/e 513 [M+H]⁺.

### Example 35

### 2-chloro-4-([(3S)-1-cyclopentyl-3-pyrrolidinyl]{[2-(trifluoromethyl)phenyl]methyl}amino)benzonitrile

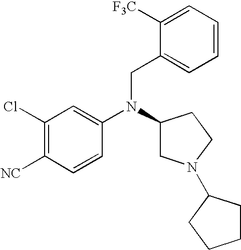

This compound was made according to general procedure of example 30 from 2-chloro-4-((3S)-3-pyrrolidinyl{[2-(trifluoromethyl)phenyl]methyl}amino)benzonitrile and cyclopentanone. LC-MS (ES) m/e 448 [M+H]⁺.

### Example 36

### 4-([(3S)-1-cyclopentyl-3-pyrrolidinyl]{[2-(trifluoromethyl)phenyl]methyl}amino)benzonitrile

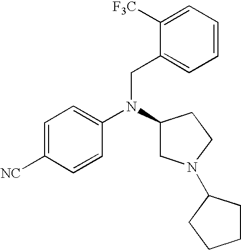

This compound was made according to general procedure of example 30 from 4-((3S)-3-pyrrolidinyl{[2-(trifluoromethyl)phenyl]methyl}amino)benzonitrile and cyclopentanone. LC-MS (ES) m/e 414 [M+H]⁺.

### Example 37

### 2-chloro-4-{[(2-chlorophenyl)methyl][(3S)-1-(1-methylethyl)-3-pyrrolidinyl]amino}benzonitrile

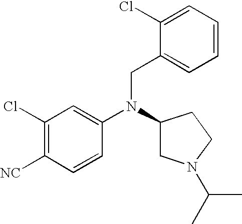

To a solution of 2-chloro-4-{[(2-chlorophenyl)methyl][(3S)-3-pyrrolidinyl]amino}benzonitrile (128 mg, 0.37 mmol) and acetone cyanohydrin (32 mg, 0.37 mmol) in 5 mL of EtOH was added 4 Å molecular sieves (crushed, activated, 500 mg). The mixture was stirred at 22° C. for 3 h and 60° C. for 2 h. The reaction mixture was cooled to 22° C. and NaBH₄ (100 mg) was added. The reaction mixture was stirred 16 h. The reaction mixture was diluted with H₂O and extracted with Et₂O. The extracts were washed with H₂O, dried over Na₂SO₄, filtered, and concentrated. The residue was purified by chromatography on an Al₂O₃ column (neutral, Brockman 2.8) (eluted with EtOAc) to yield the titled compound (140 mg, 99%). This product was converted to the HCl salt and crystallized from EtOAc, 100 mg. LC-MS (ES) m/e 388 [M+H]⁺.

### Example 38

### 2-chloro-4-{[(2-chlorophenyl)methyl][(3S)-1-(1-methyl-4-piperidinyl)-3-pyrrolidinyl]amino}benzonitrile

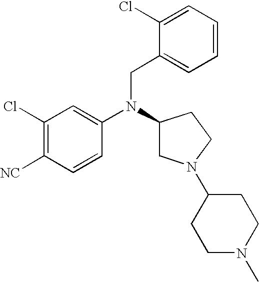

This compound was made according to general procedure of example 37 from 4-hydroxy-1-methyl-4-piperidinecarbonitrile. LC-MS (ES) m/e 443 [M+H]⁺.

### Example 39

### 2-chloro-4-([(3S)-1-(1-methylethyl)-3-pyrrolidinyl]{[2-(trifluoromethyl)phenyl]methyl}amino)benzonitrile

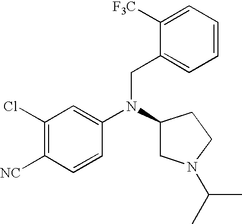

This compound was made according to general procedure of example 37 from 2-chloro-4-((3S)-3-pyrrolidinyl{[2-(trifluoromethyl)phenyl]methyl}amino)benzonitrile and acetone cyanohydrin. LC-MS (ES) m/e 422 [M+H]⁺.

### Example 40

### 4-([(3S)-1-(1-methylethyl)-3-pyrrolidinyl]{[2-(trifluoromethyl)phenyl]methyl}amino)benzonitrile

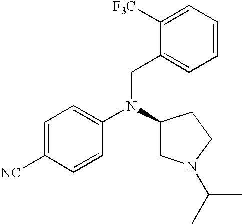

This compound was made according to general procedure of example 37 from 4-((3S)-3-pyrrolidinyl{[2-(trifluoromethyl)phenyl]methyl}amino)benzonitrile and acetone cyanohydrin. LC-MS (ES) m/e 388 [M+H]⁺.

### Example 41

### 2-chloro-4-({(3S)-1-[(5-methyl-3-isoxazolyl)methyl]-3-pyrrolidinyl}{[2-(trifluoromethyl)phenyl]methyl}amino)benzonitrile

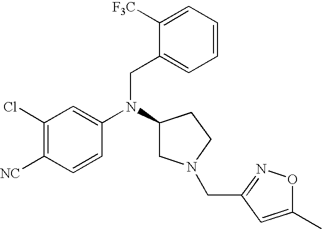

A solution of 2-chloro-4-{[(2-trifluoromethylphenyl)methyl][(3S)-3-pyrrolidinyl]amino}benzonitrile (138 mg, 0.36 mmol) and 3-(bromomethyl)-5-methylisoxazole (64 mg, 0.36 mmol) in 5 mL of acetonitrile was treated with K₂CO₃ (100 mg, 0.72 mmol), and the stirred mixture was heated to 75° C. for 15 min. The reaction mixture was concentrated, diluted with H₂O, and extracted with Et₂O. The extracts were washed with H₂O, dried over Na₂SO₄, and concentrated. The residue was purified on a small Al₂O₃ (neutral, Brockman 2.8) column (eluted with Et₂O) to yield the titled compound (114 mg, 67%). ¹H NMR (400 MHz, CDCl₃) 7.74 (d, 1H), 7.36-7.48 (m, 3H), 7.18 (d, 1H), 6.76 (d, 1H), 6.46 (m, 1H), 5.92 (s, 1H), 5.14 (d, 1H), 4.74 (d, 1H), 4.56 (m, 1H), 3.67 (d, 1H), 3.55 (d, 1H), 3.02 (m, 1H), 2.79 (m, 1H), 2.57 (m, 1H), 2.48 (m, 1H), 2.38 (m, 1H), 2.33 (s, 3H), 1.82 (m, 1H).

This product was converted to the HCl salt which crystallized from EtOAc/Et₂O and to afford 2-chloro-4-({(3S)-1-[(5-methyl-3-isoxazolyl)methyl]-3-pyrrolidinyl}{[2-(trifluoromethyl)phenyl]methyl}amino)benzonitrile hydrochloride, 90 mg. LC-MS (ES) m/c 475 [M+H]⁺.

### Example 42

### 2-chloro-4-([(3S)-1-(3-pyridinylmethyl)-3-pyrrolidinyl]{[2-(trifluoromethyl)phenyl]methyl}amino)benzonitrile

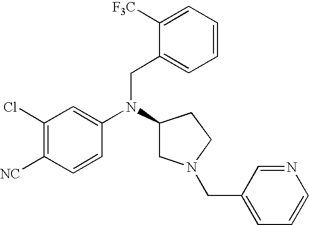

This compound was made according to general procedure of example 41 from 2-chloro-4-{[(2-trifluoromethylphenyl)methyl][(3S)-3-pyrrolidinyl]amino}benzonitrile and 3-bromomethyl pyridine in 57% yield. LC-MS (ES) m/e 471 [M+H]⁺.

### Example 43

### 2-chloro-4-({(3S)-1-[(2,6-difluorophenyl)methyl]-3-pyrrolidinyl}{[2-(trifluoromethyl)phenyl]methyl}amino)benzonitrile

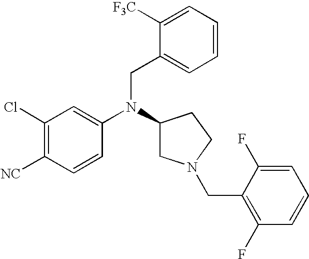

This compound was made according to general procedure of example 41 from 2-chloro-4-{[(2-trifluoromethylphenyl)methyl][(3S)-3-pyrrolidinyl]amino}benzonitrile and 2,6-difluorobenzyl bromide. LC-MS (ES) m/e 506 [M+H]⁺.

### Example 44

### 2-chloro-4-([(3S)-1-(1,3-dioxolan-2-ylmethyl)-3-pyrrolidinyl]{[2-(trifluoromethyl)phenyl]methyl}amino)benzonitrile

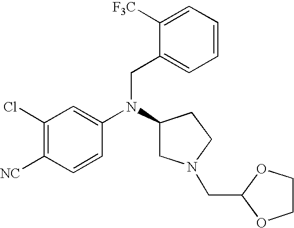

This compound was made according to general procedure of example 41 from 2-chloro-4-{[(2-trifluoromethylphenyl)methyl][(3S)-3-pyrrolidinyl]amino}benzonitrile and 2-(bromomethyl)-1,3-dioxolane. LC-MS (ES) m/e 466 [M+H]⁺.

### Example 45

### 2-chloro-4-{[(2-chlorophenyl)methyl][(3S)-1-(phenylmethyl)-3-pyrrolidinyl]amino}benzonitrile

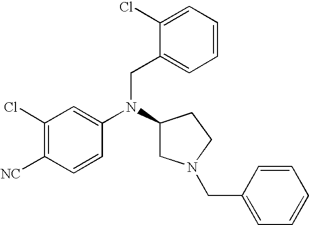

This compound was made according to general procedure of example 41 from 2-chloro-4-{[(2-chlorophenyl)methyl][(3S)-3-pyrrolidinyl]amino}benzonitrile and benzyl bromide. LC-MS (ES) m/e 436 [M+H]⁺.

### Example 46

### 2-chloro-4-{[(2-chlorophenyl)methyl][(3S)-1-(3-pyridinylmethyl)-3-pyrrolidinyl]amino}benzonitrile

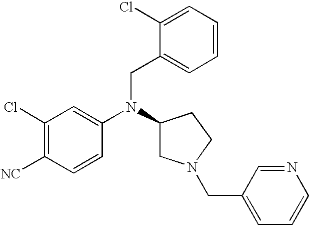

This compound was made according to general procedure of example 41 from 2-chloro-4-{[(2-chlorophenyl)methyl][(3S)-3-pyrrolidinyl]amino}benzonitrile and 3-chloromethylpyridine. LC-MS (ES) m/e 437 [M+H]⁺.

### Example 47

### 2-chloro-4-{[(2-chlorophenyl)methyl][(3S)-1-(2-pyridinylmethyl)-3-pyrrolidinyl]amino}benzonitrile

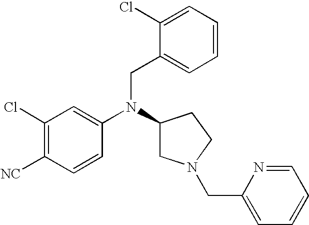

This compound was made according to general procedure of example 41 from 2-chloro-4-{[(2-chlorophenyl)methyl][(3S)-3-pyrrolidinyl]amino}benzonitrile and 2-chloromethylpyridine in 21% yield. LC-MS (ES) m/e 437 [M+H]⁺.

### Example 48

### 2-chloro-4-{[(2-chlorophenyl)methyl][(3S)-1-(4-pyridinylmethyl)-3-pyrrolidinyl]amino}benzonitrile

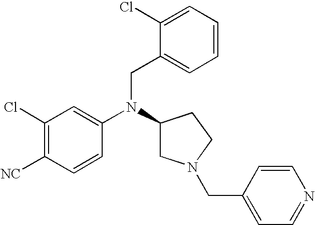

This compound was made according to general procedure of example 41 from 2-chloro-4-{[(2-chlorophenyl)methyl][(3S)-3-pyrrolidinyl]amino}benzonitrile and 4-bromomethylpyridine in 39% yield. LC-MS (ES) m/e 437 [M+H]⁺.

### Example 49

### 2-chloro-4-{[(2-chlorophenyl)methyl][(3S)-1-(3,3,3-trifluoropropyl)-3-pyrrolidinyl]amino}benzonitrile

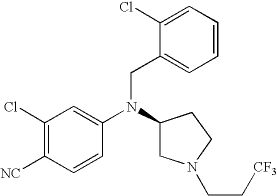

This compound was made according to general procedure of example 41 from 2-chloro-4-{[(2-chlorophenyl)methyl][(3S)-3-pyrrolidinyl]amino}benzonitrile and 1-iodo-3,3,3-trifluoropropane in 76% yield. LC-MS (ES) m/e 442 [M+H]⁺.

### Example 50

### 2-chloro-4-([(2-chlorophenyl)methyl]{(3S)-1-[2-(methyloxy)ethyl]-3-pyrrolidinyl}amino)benzonitrile

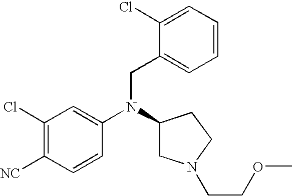

This compound was made according to general procedure of example 41 from 2-chloro-4-{[(2-chlorophenyl)methyl][(3S)-3-pyrrolidinyl]amino}benzonitrile and 1-bromo-2-methoxyethane in 80% yield. LC-MS (ES) m/e 404 [M+H]⁺.

### Example 51

### 2-chloro-4-{[(2-chlorophenyl)methyl][(3S)-1-(cyanomethyl)-3-pyrrolidinyl]amino}benzonitrile

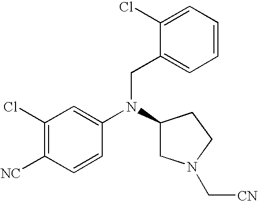

This compound was made according to general procedure of example 41 from 2-chloro-4-{[(2-chlorophenyl)methyl][(3S)-3-pyrrolidinyl]amino}benzonitrile and bromoacetonitrile in 95% yield.

LC-MS (ES) m/e 385 [M+H]⁺.

### Example 52

### ethyl((3S)-3-{(3-chloro-4-cyanophenyl)[(2-chlorophenyl)methyl]amino}-1-pyrrolidinyl)acetate

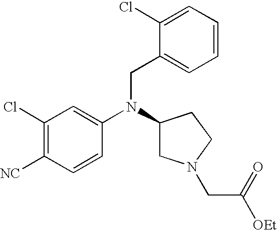

This compound was made according to general procedure of example 41 from 2-chloro-4-{[(2-chlorophenyl)methyl][(3S)-3-pyrrolidinyl]amino}benzonitrile and ethyl bromoacetate in 91% yield.

LC-MS (ES) m/e 432 [M+H]⁺.

### Example 53

### ethyl 2-((3S)-3-{(3-chloro-4-cyanophenyl)[(2-chlorophenyl)methyl]amino}-1-pyrrolidinyl)-2-methylpropanoate

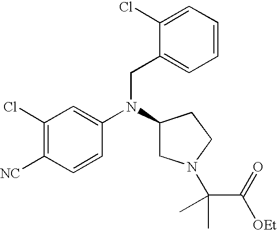

This compound was made according to general procedure of example 41 from 2-chloro-4-{[(2-chlorophenyl)methyl][(3S)-3-pyrrolidinyl]amino}benzonitrile and ethyl 2-bromo-2-methyl propionate in 15% yield. LC-MS (ES) m/e 460 [M+H]⁺.

### Example 54

### 2-((3S)-3-{(3-chloro-4-cyanophenyl)[(2-chlorophenyl)methyl]amino}-1-pyrrolidinyl)acetamide

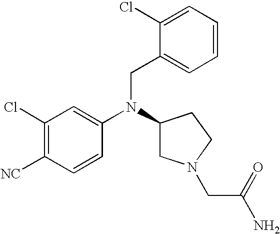

This compound was made according to general procedure of example 41 from 2-chloro-4-{[(2-chlorophenyl)methyl][(3S)-3-pyrrolidinyl]amino}benzonitrile and 2-bromoacetamide in 30% yield.

LC-MS (ES) m/e 403 [M+H]⁺.

### Example 55

### 2-((3S)-3-{(3-chloro-4-cyanophenyl)[(2-chlorophenyl)methyl]amino}-1-pyrrolidinyl)-N,N-dimethylacetamide

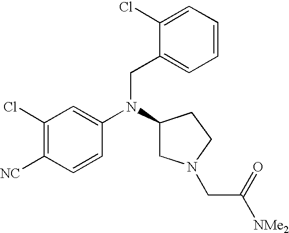

This compound was made according to general procedure of example 41 from 2-chloro-4-{[(2-chlorophenyl)methyl][(3S)-3-pyrrolidinyl]amino}benzonitrile and α-chloro-N,N-dimethylacetamide in 52% yield. LC-MS (ES) m/e 431 [M+H]⁺.

### Example 56

### 2-chloro-4-{[(2-chlorophenyl)methyl][(3S)-1-(2-oxopropyl)-3-pyrrolidinyl]amino}benzonitrile

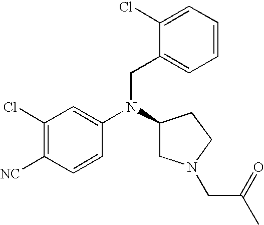

This compound was made according to general procedure of example 41 from 2-chloro-4-{[(2-chlorophenyl)methyl][(3S)-3-pyrrolidinyl]amino}benzonitrile and chloroacetone. LC-MS (ES) m/e 402 [M+H]⁺.

### Example 57

### 4-{[(2-chlorophenyl)methyl][(3S)-1-(phenylmethyl)-3-pyrrolidinyl]amino}-2-(trifluoromethyl)benzonitrile

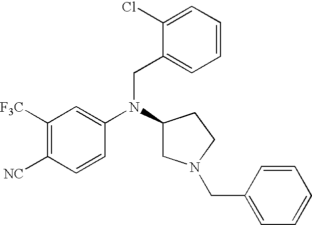

This compound was made according to general procedure of example 41 from 4-{[(2-chlorophenyl)methyl][(3S)-3-pyrrolidinyl]amino}-2-(trifluoromethyl)benzonitrile and benzyl bromide in 89% yield. LC-MS (ES) m/e 470 [M+H]⁺.

### Example 58

### 4-{[(2-chlorophenyl)methyl][(3S)-1-(2-methyl-2-propen-1-yl)-3-pyrrolidinyl]amino}-2-(trifluoromethyl)benzonitrile

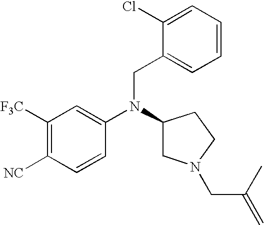

This compound was made according to general procedure of example 41 from 4-{[(2-chlorophenyl)methyl][(3S)-3-pyrrolidinyl]amino}-2-(trifluoromethyl)benzonitrile and 3-bromo-2-methyl-1-propene in 74% yield. LC-MS (ES) m/e 434 [M+H]⁺.

### Example 59

### ethyl[(3S)-3-([4-cyano-3-(trifluoromethyl)phenyl]{[2-(trifluoromethyl)phenyl]methyl}amino)-1-pyrrolidinyl]acetate

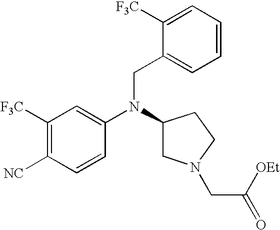

This compound was made according to general procedure of example 41 from 4-((3S)-3-pyrrolidinyl{[2-(trifluoromethyl)phenyl]methyl}amino)-2-(trifluoromethyl)benzonitrile and ethylbromoacetate in 83% yield. LC-MS (ES) m/e 500 [M+H]⁺.

### Example 60

### [(3S)-3-([4-cyano-3-(trifluoromethyl)phenyl]{[2-(trifluoromethyl)phenyl]methyl}amino)-1-pyrrolidinyl]acetic acid

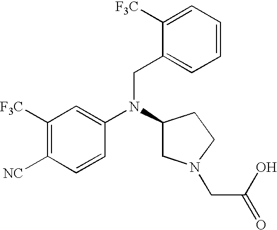

### a) 1,1-dimethylethyl [(3S)-3-([4-cyano-3-(trifluoromethyl)phenyl]{[2-(trifluoromethyl)phenyl]methyl}amino)-1-pyrrolidinyl]acetate

This compound was made according to general procedure of example 41 from 4-((3S)-3-pyrrolidinyl{[2-(trifluoromethyl)phenyl]methyl}amino)-2-(trifluoromethyl)benzonitrile and t-butyl bromoacetate in 77% yield. LC-MS (ES) m/e 528 [M+H]⁺.

### b) [(3S)-3-([4-cyano-3-(trifluoromethyl)phenyl]{[2-(trifluoromethyl)phenyl]methyl}amino)-1-pyrrolidinyl]acetic acid

To a mixture of 1,1-dimethylethyl [(3S)-3-([4-cyano-3-(trifluoromethyl)phenyl]{[2-(trifluoromethyl)phenyl]methyl}amino)-1-pyrrolidinyl]acetate (90 mg, 0.17 mmol) and CH₂Cl₂ (1 mL), TFA (1 mL) was added. The reaction was stirred for 40 minutes and then concentrated. TFA (1 mL) was then added to the residue and the reaction was stirred for 30 minutes. After concentration the residue was triturated with Et₂O to yield the titled compound as a white solid (65 mg, 65%). LC-MS (ES) m/e 472.2 [M+H]⁺.

### Example 61

### 4-({(3S)-1-[2-(methyloxy)ethyl]-3-pyrrolidinyl}{[2-(trifluoromethyl)phenyl]methyl}amino)-2-(trifluoromethyl)benzonitrile

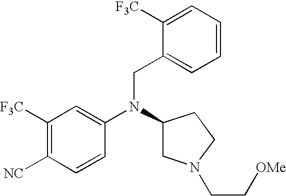

This compound was made according to general procedure of example 41 from 4-((3S)-3-pyrrolidinyl{[2-(trifluoromethyl)phenyl]methyl}amino)-2-(trifluoromethyl)benzonitrile and 1-bromo-2-methoxyethane in 39% yield. LC-MS (ES) m/e 472 [M+H]⁺.

### Example 62

### 4-([(3S)-1-(2-pyridinylmethyl)-3-pyrrolidinyl]{[2-(trifluoromethyl)phenyl]methyl}amino)benzonitrile

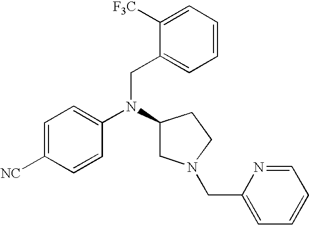

This compound was made according to general procedure of example 41 from 4-((3S)-3-pyrrolidinyl{[2-(trifluoromethyl)phenyl]methyl}amino)benzonitrile and 2-bromomethylpyridine in 65% yield. LC-MS (ES) m/e 437 [M+H]⁺.

### Example 63

### 4-([(3S)-1-(3-pyridinylmethyl)-3-pyrrolidinyl]{[2-(trifluoromethyl)phenyl]methyl}amino)benzonitrile

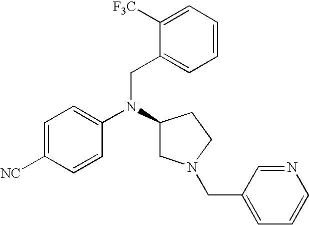

This compound was made according to general procedure of example 41 from 4-((3S)-3-pyrrolidinyl{[2-(trifluoromethyl)phenyl]methyl}amino)benzonitrile and 3-bromomethylpyridine in 43% yield. LC-MS (ES) m/e 437 [M+H]⁺.

### Example 64

### 4-([(3S)-1-(4-pyridinylmethyl)-3-pyrrolidinyl]{[2-(trifluoromethyl)phenyl]methyl}amino)benzonitrile

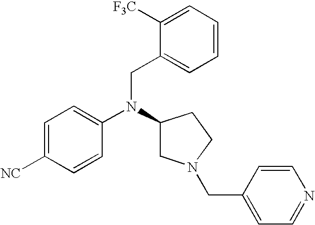

This compound was made according to general procedure of example 41 from 4-((3S)-3-pyrrolidinyl{[2-(trifluoromethyl)phenyl]methyl}amino)benzonitrile and 4-bromomethylpyridine in 64% yield. LC-MS (ES) m/e 437 [M+H]⁺.

### Example 65

### 4-([(3S)-1-(cyanomethyl)-3-pyrrolidinyl]{[2-(trifluoromethyl)phenyl]methyl}amino)benzonitrile

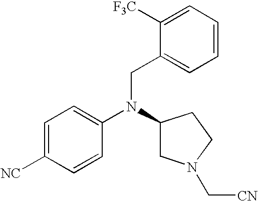

This compound was made according to general procedure of example 41 from 4-((3S)-3-pyrrolidinyl{[2-(trifluoromethyl)phenyl]methyl}amino)benzonitrile and bromoacetonitrile in 75% yield. LC-MS (ES) m/e 385 [M+H]⁺.

### Example 66

### 4-{{[2-(trifluoromethyl)phenyl]methyl}[(3S)-1-(3,3,3-trifluoropropyl)-3-pyrrolidinyl]amino}benzonitrile

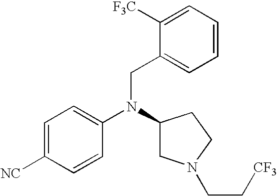

This compound was made according to general procedure of example 41 from 4-((3S)-3-pyrrolidinyl{[2-(trifluoromethyl)phenyl]methyl}amino)benzonitrile and 1-iodo-3,3,3-trifluoroethane in 92% yield. LC-MS (ES) m/e 442 [M+H]⁺.

### Example 67

### ethyl 2-[(3S)-3-((4-cyanophenyl){[2-(trifluoromethyl)phenyl]methyl}amino)-1-pyrrolidinyl]-2-methylpropanoate

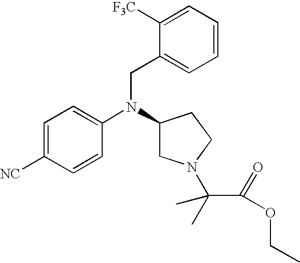

This compound was made according to general procedure of example 41 from 4-((3S)-3-pyrrolidinyl{[2-(trifluoromethyl)phenyl]methyl}amino)benzonitrile and ethyl 2-bromo-2-methyl propionate in 28% yield. LC-MS (ES) m/e 460 [M+H]⁺.

### Example 68

### 2-chloro-4-{[(3S)-1-(cyanomethyl)-3-pyrrolidinyl][(2-methylphenyl)methyl]amino}benzonitrile

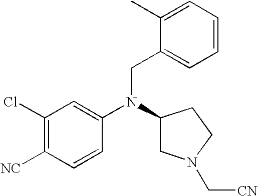

This compound was made according to general procedure of example 41 from 2-chloro-4-{[(2-methylphenyl)methyl][(3S)-3-pyrrolidinyl]amino}benzonitrile and bromoacetonitrile in 92% yield.

LC-MS (ES) m/e 365.4 [M+H]⁺.

### Example 69

### 2-chloro-4-([2-methylphenyl)methyl]{(3S)-1-[(2-methylphenyl)methyl]-3-pyrrolidinyl}amino)benzonitrile

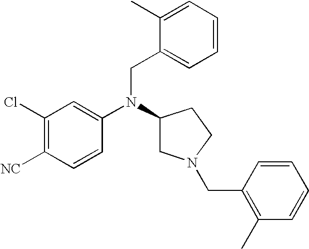

This compound was made according to general procedure of example 41 from 2-chloro-4-{[(2-methylphenyl)methyl][(3S)-3-pyrrolidinyl]amino}benzonitrile and 1-(bromomethyl)-2-methylbenzene in 92% yield. LC-MS (ES) m/e 430.2 [M+H]⁺.

### Example 70

### 2-chloro-4-{{(3S)-1-[(2-chlorophenyl)methyl]-3-pyrrolidinyl}[(2-methylphenyl)methyl]amino}benzonitrile

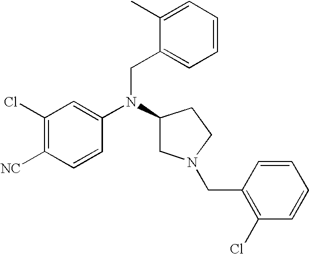

This compound was made according to general procedure of example 41 from 2-chloro-4-{[(2-methylphenyl)methyl][(3S)-3-pyrrolidinyl]amino}benzonitrile and 1-(bromomethyl)-2-chlorobenzene in 92% yield. LC-MS (ES) m/e 450.4 [M+H]⁺.

### Example 71

### 2-chloro-4-{{(3S)-1-[(3-chlorophenyl)methyl]-3-pyrrolidinyl}[(2-methylphenyl)methyl]amino}benzonitrile

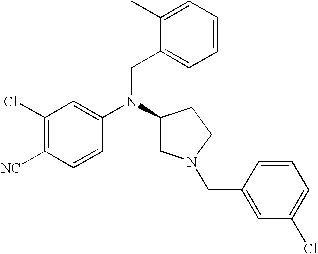

This compound was made according to general procedure of example 41 from 2-chloro-4-{[(2-methylphenyl)methyl][(3S)-3-pyrrolidinyl]amino}benzonitrile and 1-(bromomethyl)-3-chlorobenzene. LC-MS (ES) m/e 450.6 [M+H]⁺.

### Example 72

### 2-chloro-4-{((3S)-1-{[3-methyloxy)phenyl]methyl}-3-pyrrolidinyl)[2-methylphenyl)methyl]amino}benzonitrile

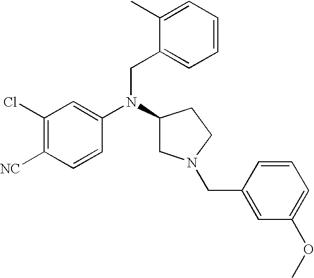

This compound was made according to general procedure of example 41 from 2-chloro-4-{[(2-methylphenyl)methyl][(3S)-3-pyrrolidinyl]amino}benzonitrile and 1-(bromomethyl)-3-(methyloxy)benzene. LC-MS (ES) m/e 446.6 [M+H]⁺.

### Example 73

### 2-chloro-4-{{(3S)-1-[(4-chlorophenyl)methyl]-3-pyrrolidinyl}[(2-methylphenyl)methyl]amino}benzonitrile

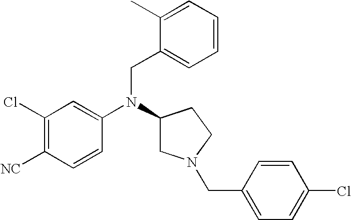

This compound was made according to general procedure of example 41 from 2-chloro-4-{[(2-methylphenyl)methyl][(3S)-3-pyrrolidinyl]amino}benzonitrile and 1-(bromomethyl)-4-chlorobenzene. LC-MS (ES) m/e 450.4 [M+H]⁺.

### Example 74

### 1,1-dimethylethyl 3-[(3S)-3-((3-chloro-4-cyanophenyl){[2-(trifluoromethyl)phenyl]methyl}amino)-1-pyrrolidinyl]propanoate

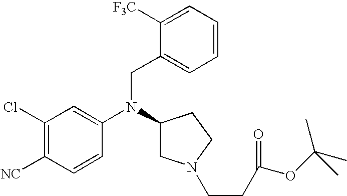

A solution of 2-chloro-4-((3S)-3-pyrrolidinyl{[2-(trifluoromethyl)phenyl]methyl}amino)benzonitrile (270 mg, 0.71 mmol) and t-butyl acrylate (91 mg, 0.71 mmol) in 5 mL of MeOH was treated with 1 drop of 3.87 M NaOMc in MeOH, and the reaction was stirred 72 h. The reaction mixture was diluted with H₂O and extracted with Et₂O. The extracts were washed with H₂O, dried over Na₂SO₄, filtered, and concentrated. The residue was purified by column chromatography (eluted with 15% EtOAc/hexane) to yield the titled compound (200 mg, 56%).

LC-MS (ES) m/e 508 [M+H]⁺.

### Example 75

### 4-([(3S)-1-(2-hydroxyethyl)-3-pyrrolidinyl]{[2-(trifluoromethyl)phenyl]methyl}amino)-2-(trifluoromethyl)benzonitrile

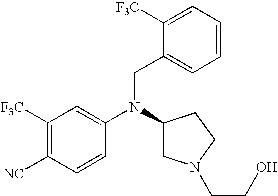

A solution of ethyl [(3S)-3-([4-cyano-3-(trifluoromethyl)phenyl]{[2-(trifluoromethyl)phenyl]methyl}amino)-1-pyrrolidinyl]acetate (170 mg, 0.34 mmol) in 5 mL of Et₂O was added to a suspension of LiAlH₄ (200 mg) in 20 mL of Et₂O at −20° C. After 5 min, the reaction was quenched by the consecutive addition of 0.2 mL of H₂O, 0.2 mL of 15% NaOH and 0.6 mL of H₂O. After stirring at 22° C. for 15 min, the mixture was filtered and the filtrate concentrated. The residue was purified by column chromatography on a Florisil® column (eluted with 2% MeOH/CH₂Cl₂) to yield the titled compound (90 mg, 56%). LC-MS (ES) m/e 458 [M+H]¹.

### Example 76

### 4-{[(2-chlorophenyl)methyl][(3S)-1-(2-methylpropyl)-3-pyrrolidinyl]amino}-2-(trifluoromethyl)benzonitrile

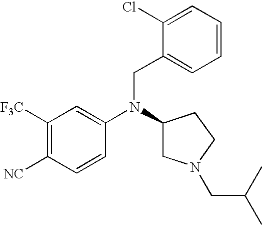

A solution of 4-{[(2-chlorophenyl)methyl][(3S)-1-(2-methyl-2-propen-1-yl)-3-pyrrolidinyl]amino}-2-(trifluoromethyl)benzonitrile hydrochloride (105 mg, 0.22 mmol) in 2 mL of EtOH and 2 mL of MeOH was treated with PtO₂ and hydrogenated at 1 atmosphere H₂ pressure for 15 min. The reaction mixture was filtered (to remove the catalyst) and concentrated. The residue was converted to the free base and purified by column chromatography (eluted with 25% EtOAc/hexane) on an Al₂O₃ column (neutral, activity 1) to yield the titled compound (43 mg, 41%). LC-MS (ES) m/e 436 [M+H]⁺.

### Example 77

### 2-chloro-4-{[(2-methylphenyl)methyl][(3S)-1-methylsulfonyl)-3-pyrrolidinyl]amino}benzonitrile

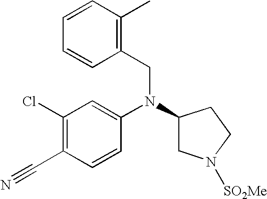

To a solution of 2-chloro-4-{[(2-methylphenyl)methyl][(3S)-3-pyrrolidinyl]amino}benzonitrile (1.8 g, 5.5 mmol) in anhydrous CH₂Cl₂ was added pyridine (0.90 mL, 11.0 mmol) followed by methanesulfonyl chloride (0.47 mL, 6.0 mmol). The reaction mixture stirred at RT for 2 h. The crude, dark brown mixture was diluted with CH₂Cl₂ and washed with 1N HCl (3×50 mL). The organic phase was concentrated, and the residue was purified by column chromatography (silica gel 60, EMD Chemicals) (using a gradient of 30-60% EtOAc:hexanes) to yield the titled compound (1.4 g, 61%) as a white solid. LC-MS (ESI) 404.2 [M+H]⁺.

### Example 78

### 2-chloro-4-{{[4-fluoro-2-(trifluoromethyl)phenyl]methyl}[(3S)-1-methyl-3-pyrrolidinyl]amino}benzonitrile

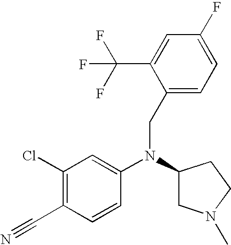

### a) 2-chloro-4-[(3S)-3-pyrrolidinylamino]benzonitrile

To a solution of 1,1-dimethylethyl (3S)-3-[(3-chloro-4-cyanophenyl)amino]-1-pyrrolidinecarboxylate (6.0 g, 18.7 mmol) in CH₂Cl₂ (40 mL) was added TFA (9.2 mL, 148 mmol). The reaction mixture was stirred for 5 h. Toluene (20 mL) was added to reaction mixture and then the mixture was concentrated. Saturated NaHCO₃ (50 mL) was added to the residue and the mixture was extracted with EtOAc (5×100 mL). The organic extracts were dried over MgSO₄, filtered, and concentrated to yield the crude product as a red-brown solid (6.8 g). LC-MS (ES) m/e 222.0 [M+H]⁺.

### b) 2-chloro-4-{[(3S)-1-methyl-3-pyrrolidinyl]amino}benzonitrile

To a solution of 2-chloro-4-[(3S)-3-pyrrolidinylamino]benzonitrile (2.0 g, 9 mmol) in MeOH (100 mL) was added formaldehyde (0.7 mL, 9 mmol). After reaction mixture was stirred for 1 h, NaBH₄ (1.02 g, 27 mmol) was added. After reaction was stirred overnight, it was quenched with H₂O (15 mL). The mixture was concentrated to aqueous solution and extracted with CH₂Cl₂ (4×100 mL). The organic extracts were dried over MgSO₄, filtered, concentrated, and purified via column chromatography to yield the titled compound (2.02 g, 75%) as a brown oil. LC-MS (ES) m/e 236.0[M+H]⁺.

### c) 2-chloro-4-{{[4-fluoro-2-(trifluoromethyl)phenyl]methyl}[(3S)-1-methyl-3-pyrrolidinyl]amino}benzonitrile

To a solution of 2-chloro-4-{[(3S)-1-methyl-3-pyrrolidinyl]amino}benzonitrile (120 mg, 0.5 mmol) in DMF (2 mL) was added NaH (60% in mineral oil, 93 mg, 1.75 mmol). Reaction was stirred for 1 h and then 4-fluoro-2-(trifluoromethyl)benzyl bromide (129 mg, 0.5 mmol) in DMF (0.5 mL) was added dropwise. Reaction was stirred for 2 h and extracted with EtOAc (12 mL) and H₂O (4 mL). The organic layer was washed with saturated NaHCO₃ (4×5 mL). The organic layer was then dried with MgSO₄, filtered, and concentrated. The residue was purified via column chromatography (eluted with EtOAc and hexane, 1:1) to yield the titled compound (87 mg, 45%) as a brown oil. LC-MS (ES) m/e 413.0 [M+H]⁺.

### Example 79

### 2-chloro-4-{[(3-fluorophenyl)methyl][(3S)-1-methyl-3-pyrrolidinyl]amino}benzonitrile

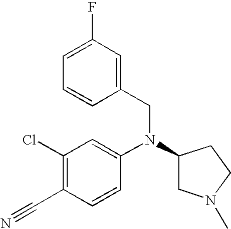

This compound was made according to general procedure of example 78 from 3-fluorobenzyl bromide. LC-MS (ES) m/e 344.2 [M+H]⁺.

### Example 80

### 2-chloro-4-{[(3-chlorophenyl)methyl][(3S)-1-methyl-3-pyrrolidinyl]amino}benzonitrile

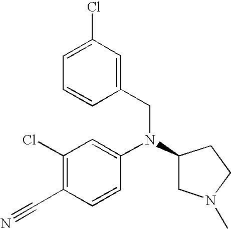

This compound was made according to general procedure of example 78 from 3-chlorobenzyl bromide. LC-MS (ES) m/e 360.0 [M+H]⁺.

### Example 81

### 2-chloro-4-{[(2-fluoro-3-methylphenyl)methyl][(3S)-1-methyl-3-pyrrolidinyl]amino}benzonitrile

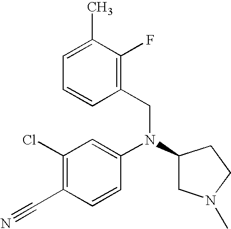

This compound was made according to general procedure of example 78 from 1-(bromomethyl)-2-fluoro-3-methylbenzene. LC-MS (ES) m/e 358.4 [M+H]⁺.

### Example 82

### 2-chloro-4-{[(3-methylphenyl)methyl][(3S)-1-methyl-3-pyrrolidinyl]-amino}benzonitrile

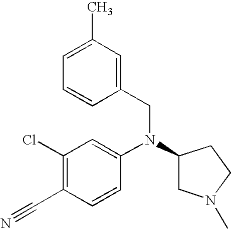

This compound was made according to general procedure of example 78 from 1-(bromomethyl)-3-methylbenzene. LC-MS (ES) m/c 341.4 [M+H]⁺.

### Example 83

### 2-chloro-4-{{[3-fluoro-2-(trifluoromethyl)phenyl]methyl}[(3S)-1-methyl-3-pyrrolidinyl]amino}benzonitrile

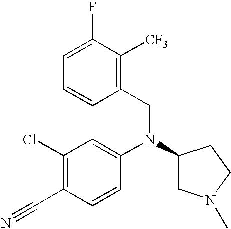

This compound was made according to general procedure of example 78 from 1-(bromomethyl)-3-fluoro-2-(trifluoromethyl)benzene. LC-MS (ES) m/e 412.4 [M+H]⁺.

### Example 84

### 2-chloro-4-{{[2-chloro-3-(trifluoromethyl)phenyl]methyl}[(3S)-1-methyl-3-pyrrolidinyl]amino}benzonitrile

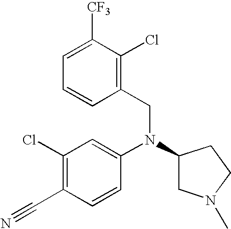

This compound was made according to general procedure of example 78 from 1-(bromomethyl)-2-chloro-3-(trifluoromethyl)benzene. LC-MS (ES) m/e 428.0 [M+H]⁺.

### Example 85

### 2-chloro-4-([(3S)-1-methyl-3-pyrrolidinyl]{[4-(trifluoromethyl)phenyl]methyl}amino)benzonitrile

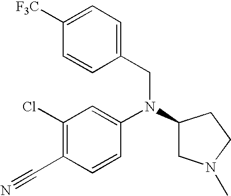

This compound was made according to general procedure of example 78 from 1-(bromomethyl)-4-(trifluoromethyl)benzene. LC-MS (ES) m/e 394.4 [M+H]⁺.

### Example 86

### 4-{(2,1,3-benzothiadiazol-5-ylmethyl)[(3S)-1-methyl-3-pyrrolidinyl]amino}-2-chlorobenzonitrile

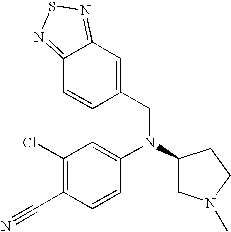

This compound was made according to general procedure of example 78 from 5-(bromomethyl)-2,1,3-benzothiadiazole. LC-MS (ES) m/c 384 [M+H]⁺.

### Example 87

### 2-chloro-4-{{[2-fluoro-3-(trifluoromethyl)phenyl]methyl}[(3S)-1-methyl-3-pyrrolidinyl]amino}benzonitrile

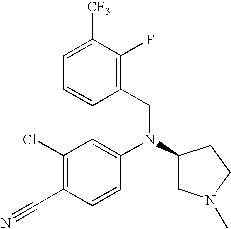

This compound was made according to general procedure of example 78 from 1-(bromomethyl)-2-fluoro-3-(trifluoromethyl)benzene. LC-MS (ES) m/e 413.0 [M+H]⁺.

### Example 88

### 2-chloro-4-([(3S)-1-methyl-3-pyrrolidinyl]{[3-(trifluoromethyl)phenyl]methyl}amino)benzonitrile

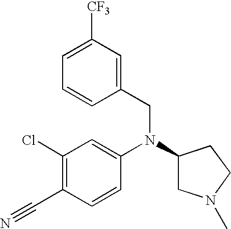

This compound was made according to general procedure of example 78 from 1-(bromomethyl)-3-(trifluoromethyl)benzene. LC-MS (ES) m/e 394.0 [M+H]⁺.

### Example 89

### 2-chloro-4-{ethyl[(3S)-1-methyl-3-pyrrolidinyl]amino}benzonitrile

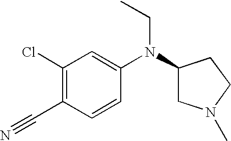

This compound was made according to general procedure of example 78 from iodoethane. LC-MS (ES) m/e 264.0 [M+H]⁺.

### Example 90

### 2-chloro-4-{[(3-cyanophenyl)methyl][(3S)-1-methyl-3-pyrrolidinyl]amino}benzonitrile

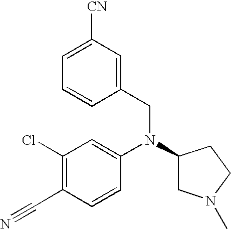

This compound was made according to general procedure of example 78 from 3-(bromomethyl)benzonitrile. LC-MS (ES) m/e 351.6 [M+H]⁺.

### Example 91

### 2-chloro-4-{[(4-chlorophenyl)methyl][(3S)-1-methyl-3-pyrrolidinyl]amino}benzonitrile

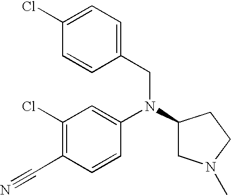

This compound was made according to general procedure of example 78 from 1-(bromomethyl)-4-chlorobenzene. LC-MS (ES) m/e 360 [M+H]⁺.

### Example 92

### 4-{[(6-bromo-1,3-benzodioxol-5-yl)methyl][(3S)-1-methyl-3-pyrrolidinyl]amino}-2-chlorobenzonitrile

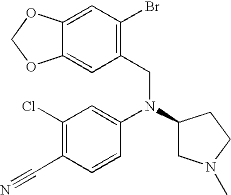

This compound was made according to general procedure of example 78 from 5-bromo-6-(bromomethyl)-1,3-benzodioxole. LC-MS (ES) m/e 450.0 [M+H]⁺.

### Example 93

### 2-chloro-4-{(2-methyl-2-propen-1-yl)[(3S)-1-methyl-3-pyrrolidinyl]amino}benzonitrile

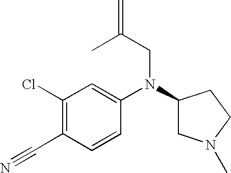

This compound was made according to general procedure of example 78 from 3-bromo-2-methyl-1-propene. LC-MS (ES) m/e 290.0 [M+H]⁺.

### Example 94

### 2-chloro-4-{[(2-chloro-5-fluorophenyl)methyl][(3S)-1-methyl-3-pyrrolidinyl]amino}benzonitrile

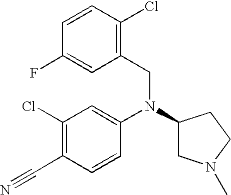

This compound was made according to general procedure of example 78 from 2-(bromomethyl)-1-chloro-4-fluorobenzene. LC-MS (ES) m/e 379 [M+H]⁺.

### Example 95

### 2-chloro-4-{[(5-methyl-3-isoxazolyl)methyl][(3S)-1-methyl-3-pyrrolidinyl]amino}benzonitrile

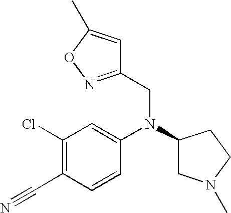

This compound was made according to general procedure of example 78 from 3-(bromomethyl)-5-methylisoxazole. LC-MS (ES) m/e 331 [M+H]⁺.

### Example 96

### 4-{[(2-bromophenyl)methyl][(3S)-1-methyl-3-pyrrolidinyl]amino}-2-chlorobenzonitrile

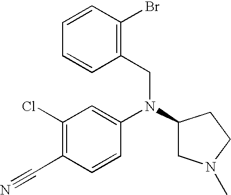

This compound was made according to general procedure of example 78 from 1-bromo-2-(bromomethyl)benzene. LC-MS (ES) m/e 404 [M]⁺.

### Example 97

### 2-chloro-4-([(3S)-1-methyl-3-pyrrolidinyl]{[5-(trifluoromethyl)-3-furanyl]methyl}amino)benzonitrile

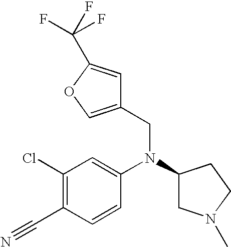

This compound was made according to general procedure of example 78 from 4-(bromomethyl)-2-(trifluoromethyl)furan. LC-MS (ES) m/c 384 [M+H]⁺.

### Example 98

### 2-chloro-4-{[(2-chloro-6-fluorophenyl)methyl][(3S)-1-methyl-3-pyrrolidinyl]amino}benzonitrile

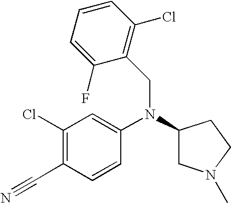

This compound was made according to general procedure of example 78 from 2-(bromomethyl)-1-chloro-3-fluorobenzene. LC-MS (ES) m/e 378 [M+H]⁺.

### Example 99

### 2-chloro-4-{(cyclohexylmethyl)[(3S)-1-methyl-3-pyrrolidinyl]-amino}benzonitrile

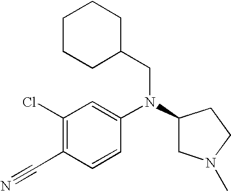

This compound was made according to general procedure of example 78 from (bromomethyl)cyclohexane. LC-MS (ES) m/e 331.6 [M]⁺.

### Example 100

### 4-{[(3-bromophenyl)methyl][(3S)-1-methyl-3-pyrrolidinyl]amino}-2-chlorobenzonitrile

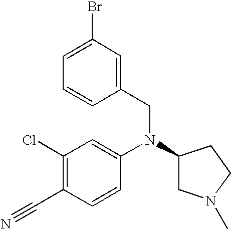

This compound was made according to general procedure of example 78 from 1-bromo-3-(bromomethyl)benzene. LC-MS (ES) m/e 404.2 [M]⁺.

### Example 101

### 2-chloro-4-{{[2-chloro-3,4-bis(methyloxy)phenyl]methyl}[(3S)-1-methyl-3-pyrrolidinyl]amino}benzonitrile

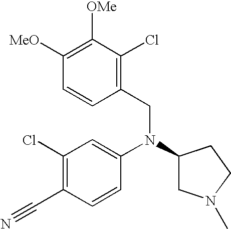

This compound was made according to general procedure of example 78 from 1-(bromomethyl)-2-chloro-3,4-bis(methyloxy)benzene. LC-MS (ES) m/e 422 [M+H]⁺.

### Example 102

### 2-chloro-4-[[(3S)-1-methyl-3-pyrrolidinyl](2-propen-1-yl)amino]benzonitrile

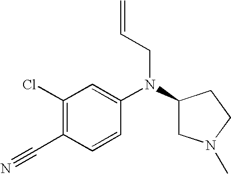

This compound was made according to general procedure of example 78 from 3-bromo-1-propene.

LC-MS (ES) m/e 276 [M+H]⁺.

### Example 103

### 2-chloro-4-[[(3S)-1-methyl-3-pyrrolidinyl](2-naphthalenylmethyl)amino]benzonitrile

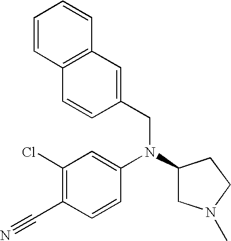

This compound was made according to general procedure of example 78 from 2-(bromomethyl)naphthalene. LC-MS (ES) m/e 376 [M+H]⁺.

### Example 104

### 4-{butyl[(3S)-1-methyl-3-pyrrolidinyl]amino}-2-chlorobenzonitrile

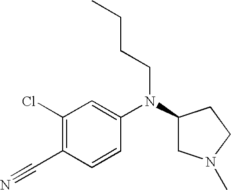

This compound was made according to general procedure of example 78 from 1-bromobutane.

LC-MS (ES) m/e 292 [M+H]⁺.

### Example 105

### 2-chloro-4-{[(2-chlorophenyl)methyl][(3S)-1-(methylsulfonyl)-3-pyrrolidinyl]amino}benzonitrile

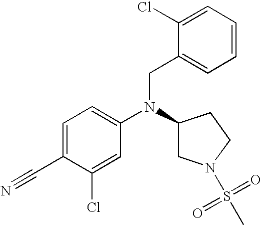

### a) 2-chloro-4-{[(3S)-1-(methylsulfonyl)-3-pyrrolidinyl]amino}benzonitrile

Methanesulfonyl chloride (0.52 g, 4.57 mmol) was added to a solution of 2-chloro-4-[(3S)-3-pyrrolidinylamino]benzonitrile (1.15 g, 3.81 mmol) and TEA (1.53 g, 15.24 mmol) in CH₂Cl₂ (15 mL) at 0° C. The reaction mixture was stirred at RT for 2 h and then partitioned between H₂O and CH₂Cl₂. The organic layer was dried over Na₂SO₄, filtered, and concentrated. The residue was purified by column chromatography (eluted with 0% EtOAc in CH₂Cl₂ grading to 17% EtOAc in CH₂Cl₂) to yield the titled compound (0.93 g, 81%) as a white foam. LC-MS (ES) m/e 300.4 [M+H]¹.

### b) 2-chloro-4-{[(2-chlorophenyl)methyl][(3S)-1-(methylsulfonyl)-3-pyrrolidinyl]amino}benzonitrile

NaH (60% in mineral oil, 0.035 g, 0.875 mmol) was added to a solution of 2-chloro-4-{[(3S)-1-(methylsulfonyl)-3-pyrrolidinyl]amino}benzonitrile (0.068 g, 0.226 mmol) in DMF (3 mL) at 0° C. under a N₂ atmosphere. After stirring for 30 min, 1-(bromomethyl)-2-chlorobenzene (0.049 g, 0.238 mmol) was added and the reaction mixture was stirred at RT for 3 h. The reaction mixture was quenched with H₂O and then partitioned between EtOAc and H₂O. The organic layer was dried over Na₂SO₄, filtered, and concentrated. The residue was purified via column chromatography (with 0% EtOAc in hexane grading to 45% EtOAc in hexane) to yield the titled compound (0.074 g, 77%) as a light brown solid. LC-MS (ES) m/c 424.4 [M+H]¹.

### Example 106

### 2-chloro-4-([(3S)-1-(methylsulfonyl)-3-pyrrolidinyl]{[2(trifluoromethyl)phenyl]methyl}amino)benzonitrile

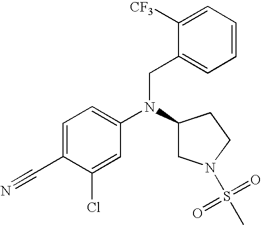

This compound was made according to general procedure of example 105 from 1-(bromomethyl)-2-(trifluoromethyl)benzene. LC-MS (ES) m/e 458.4 [M+H]⁺.

### Example 107

### 4-{[(2-bromophenyl)methyl][(3S)-1-(methylsulfonyl)-3-pyrrolidinyl]amino}-2-chlorobenzonitrile

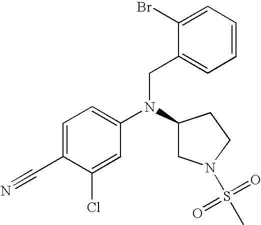

This compound was made according to general procedure of example 105 from 1-bromo-2-(bromomethyl)benzene. LC-MS (ES) m/e 467.4 [M]⁺.

### Example 108

### 2-chloro-4-{(2-methyl-2-propen-1-yl)[(3S)-1-(methylsulfonyl)-3-pyrrolidinyl]amino}benzonitrile

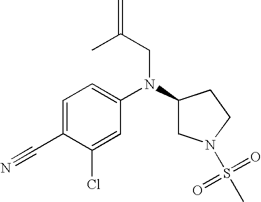

This compound was made according to general procedure of example 105 from 3-bromo-2-methyl-1-propene. LC-MS (ES) m/e 354.4 [M+H]⁺.

### Example 109

### 2-chloro-4-{ethyl[(3S)-1-(methylsulfonyl)-3-pyrrolidinyl]amino}benzonitrile

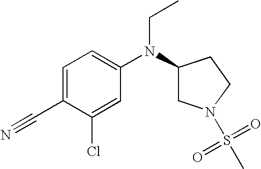

This compound was made according to general procedure of example 105 from iodoethane. LC-MS (ES) m/e 328.2 [M+H]⁺.

### Example 110

### 2-chloro-4-{[(2-fluorophenyl)methyl][(3S)-1-(methylsulfonyl)-3-pyrrolidinyl]amino}benzonitrile

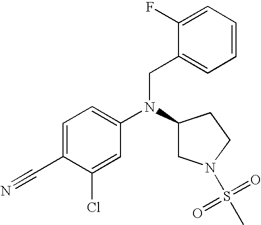

This compound was made according to general procedure of example 105 from 1-(bromomethyl)-2-fluorobenzene. LC-MS (ES) m/e 408.6 [M+H]⁺.

### Example 111

### 2-chloro-4-[[(3S)-1-(methylsulfonyl)-3-pyrrolidinyl](2-thienylmethyl)amino]benzonitrile

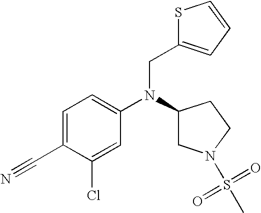

This compound was made according to general procedure of example 105 from 2-(bromomethyl)thiophene. LC-MS (ES) m/e 396.4 [M+H]⁺.

### Example 112

### 2-chloro-4-{(2-furanylmethyl)[(3S)-1-(methylsulfonyl)-3-pyrrolidinyl]amino}benzonitrile

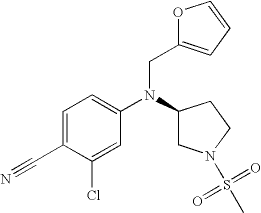

This compound was made according to general procedure of example 105 from 2-(bromomethyl)furan. LC-MS (ES) m/e 380.4 [M+H]⁺.

### Example 113

### 2-chloro-4-([(3S)-1-(methylsulfonyl)-3-pyrrolidinyl]{[3-(trifluoromethyl)phenyl]methyl}amino)benzonitrile

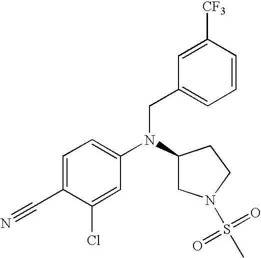

This compound was made according to general procedure of example 105 from 1-(bromomethyl)-3-(trifluoromethyl)benzene. LC-MS (ES) m/e 458.4 [M+H]⁺.

### Example 114

### 2-chloro-4-{[(3S)-1-(methylsulfonyl)-3-pyrrolidinyl][(3-methyl-2-thienyl)methyl]amino}benzonitrile

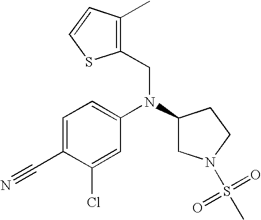

This compound was made according to general procedure of example 105 from 2-(bromomethyl)-3-methylthiophene. LC-MS (ES) m/e 410.2 [M+H]⁺.

### Example 115

### 2-chloro-4-{(3-furanylmethyl)[(3S)-1-(methylsulfonyl)-3-pyrrolidinyl]amino}benzonitrile

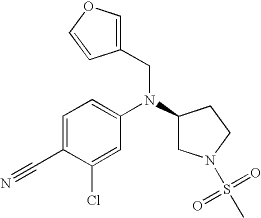

This compound was made according to general procedure of example 105 from 3-(bromomethyl)furan. LC-MS (ES) m/e 380.4 [M+H]⁺.

### Example 116

### 2-chloro-4-{[(2-chloro-5-fluorophenyl)methyl][(3S)-1-(methylsulfonyl)-3-pyrrolidinyl]amino}benzonitrile

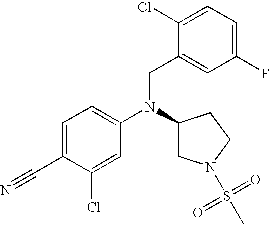

This compound was made according to general procedure of example 105 from 2-(bromomethyl)-1-chloro-4-fluorobenzene. LC-MS (ES) m/e 441.6 [M]⁺.

### Example 117

### 2-chloro-4-{[(3S)-1-(methylsulfonyl)-3-pyrrolidinyl][(5-methyl-2-thienyl)methyl]amino}benzonitrile

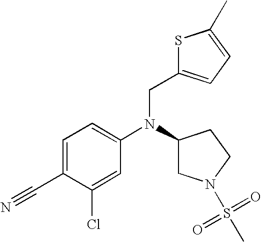

### a) (5-methyl-2-thienyl)methanol

LiAlH₄ (1M in THF, 5.6 mL) was added dropwise to a solution of 5-methyl-2-thiophenecarboxylic acid (0.4 g, 2.8 mmol) in THF (10 mL) at 0° C. under a N₂ atmosphere. After stirring for 4 h, saturated K₂CO₃ was added slowly, and the mixture was filtered through a pad of Celite. The filtrate was partitioned between EtOAc and H₂O, and the organic layer was dried over Na₂SO₄, filtered, concentrated to yield the titled compound (0.165 g, 46%) as a clear oil. ¹H-NMR (CDCl₃) δ: 2.495 (s, 3H,), 4.761 (s, 2H), 6.636 (m, 1H), 6.818 (d, 1H, J=3.2 Hz).

### b) (5-methyl-2-thienyl)methyl methanesulfonate

Methanesulfonyl chloride (0.192 g, 1.68 mmol) was added to a solution of (5-methyl-2-thienyl)methanol (0.165 g, 1.29 mmol) and TEA (0.39 g, 3.86 mmol) in CH₂Cl₂ (6 mL) at 0° C. The reaction mixture was stirred at RT for 2 h, and then partitioned between H₂O and CH₂Cl₂. The organic layer was dried over Na₂SO₄, filtered, and concentrated to yield the titled compound (0.150 g, 56%) as a brown oil. ¹H-NMR (CDCl₃) δ: 2.490 (s, 3H), 3.166 (s, 3H), 4.767 (s, 2H), 6.611 (m, 1H), 6.887 (d, 1H, J=1.6 Hz).

### c) 2-chloro-4-{[(3S)-1-(methylsulfonyl)-3-pyrrolidinyl][(5-methyl-2-thienyl)methyl]amino}benzonitrile

The title compound was prepared from (5-methyl-2-thienyl)methyl methanesulfonate using the procedure described in example 105 (part b). LC-MS (ES) m/e 410.2 [M+H]⁺.

### Example 118

### 2-chloro-4-{[(3-methyl-2-furanyl)methyl][(3S)-1-(methylsulfonyl)-3-pyrrolidinyl]amino}benzonitrile

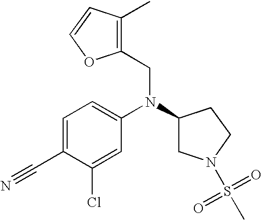

This compound was made according to general procedure of example 117 part b and c from (3-methyl-2-furanyl)methanol. LC-MS (ES) m/e 394.2 [M+H]⁺.

### Example 119

### 4-{[(5-bromo-2-thienyl)methyl][(3S)-1-methyl-3-pyrrolidinyl]amino}-2-chlorobenzonitrile

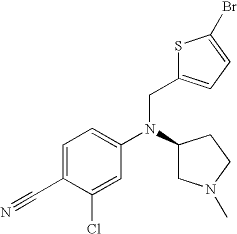

### a)-2-bromo-5-(bromomethyl)thiophene

N-bromosuccinimide (1.11 g, 6.2 mmol) was added to a solution of 2-bromo-5-methylthiophene (1 g, 5.6 mmol) in CCl₄ (35 mL), and the reaction mixture was refluxed for 16 h. After cooled down to RT, the mixture was filtered through a pad of Celite, and the filtrate was concentrated to yield the titled compound (1.3 g, 100%) as a light yellow oil. ¹H-NMR (CDCl₃) δ: 4.664 (s, 2H), 6.898 (m, 2H).

### b) 4-{[(5-bromo-2-thienyl)methyl][(3S)-1-methyl-3-pyrrolidinyl]amino}-2-chlorobenzonitrile

This compound was made according to general procedure of example 78 (part c) from 2-bromo-5-(bromomethyl)thiophene and 2-chloro-4-{[(3S)-1-methyl-3-pyrrolidinyl]amino}benzonitrile using the procedure described in example 78 (part c). LC-MS (ES) m/e 410.4 [M]⁺.

### Example 120

### 2-chloro-4-{({5-[6-methyloxy)-3-pyridinyl]-2-thienyl}methyl)[(3S)-1-methyl-3-pyrrolidinyl]amino}benzonitrile

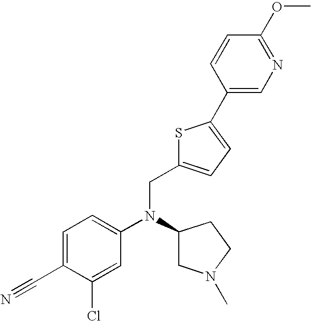

Pd(dppf)Cl₂ with CH₂Cl₂ (0.01 g, 0.0097 mmol) was added to a suspension of 4-{[(5-bromo-2-thienyl)methyl][(3S)-1-methyl-3-pyrrolidinyl]amino}-2-chlorobenzonitrile) (0.04 g, 0.097 mmol), [6-(methyloxy)-3-pyridinyl]boronic acid (0.015 g, 0.125 mmol, and K₂CO₃ (0.046 g, 0.33 mmol) in 3:1 dioxane/H₂O (2 mL). The reaction mixture was heated in the microwave at 100° C. for 10 min, and then filtered through a pad of Celite. The filtrate was partitioned between H₂O and EtOAc. The organic layer was dried over Na₂SO₄, filtered, and concentrated. The residue was purified by column chromatography (with 0% MeOH in CH₂Cl₂ grading to 4% MeOH in CH₂Cl₂) to yield the titled compound (7 mg, 16%) as a brown oil. LC-MS (ES) m/e 439.4 [M+H]⁺.

### Example 121

### 2-chloro-4-{(2-methylpropyl)[(3S)-1-methyl-3-pyrrolidinyl]-amino}benzonitrile

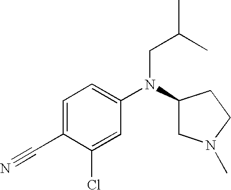

PtO₂ (83% on carbon, 10 mg) was added to a degassed solution of 2-chloro-4-{(2-methyl-2-propen-1-yl)[(3S)-1-methyl-3-pyrrolidinyl]amino}benzonitrile (0.022 g, 0.076 mmol) and 2 drops of 1M HCl in 1:1 ethanol/MeOH (3 mL). The reaction mixture was stirred at RT for 1 h under a H₂ atmosphere (1 atm), and then filtered through a pad of Celite. The filtrate was concentrated, and the residue was partitioned between EtOAc and saturated NaHCO₃. The organic layer was dried over Na₂SO₄, filtered, and concentrated. The residue was purified via column chromatography (with 0% MeOH in CH₂Cl₂ grading to 8% MeOH in CH₂Cl₂) to yield the titled compound (0.022 g, 99%) as a yellow oil. LC-MS (ES) m/e 292.4 [M+H]⁺.

### Example 122

### 2-chloro-4-{[(5-fluoro-2-methylphenyl)methyl][(3S)-1-(methylsulfonyl)-3-pyrrolidinyl]amino}benzonitrile

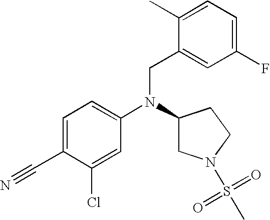

This compound was made according to general procedure of example 105 from 2-(bromomethyl)-4-fluoro-1-methylbenzene. LC-MS (ES) m/e 422.4 [M+H]⁺.

### Example 123

### 4-{{[2-bromo-4,5-bis(methyloxy)phenyl]methyl}[(3S)-1-(methylsulfonyl)-3-pyrrolidinyl]amino}-2-chlorobenzonitrile

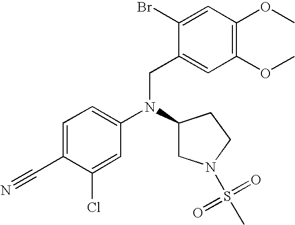

This compound was made according to general procedure of example 105 from 1-bromo-2-(bromomethyl)-4,5-bis(methyloxy)benzene. LC-MS (ES) m/e 528.4 [M]⁺.

### Example 124

### 2-chloro-4-{{[3-fluoro-2-(trifluoromethyl)phenyl]methyl}[(3S)-1-(methylsulfonyl)-3-pyrrolidinyl]amino}benzonitrile

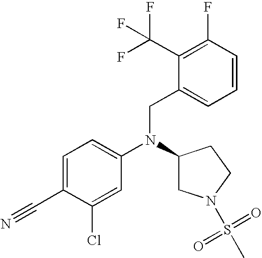

This compound was made according to general procedure of example 105 from 1-(bromomethyl)-3-fluoro-2-(trifluoromethyl)benzene. LC-MS (ES) m/e 475.0 [M]⁺.

### Example 125

### 2-chloro-4-{[(2,5-dichlorophenyl)methyl][(3S)-1-(methylsulfonyl)-3-pyrrolidinyl]amino}benzonitrile

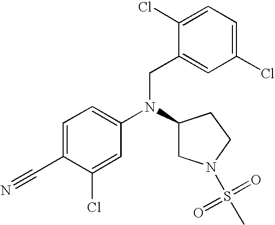

This compound was made according to general procedure of example 105 from 2-(bromomethyl)-1,4-dichlorobenzene. LC-MS (ES) m/e 458.4 [M+H]⁺.

### Example 126

### 2-chloro-4-{[(2-cyanophenyl)methyl][(3S)-1-(methylsulfonyl)-3-pyrrolidinyl]amino}benzonitrile

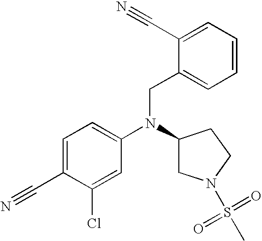

This compound was made according to general procedure of example 105 from 2-(bromomethyl)benzonitrile. LC-MS (ES) m/e 415.6 [M+H]⁺.

### Example 127

### 2-chloro-4-{{[4-fluoro-2-(trifluoromethyl)phenyl]methyl}[(3S)-1-(methylsulfonyl)-3-pyrrolidinyl]amino}benzonitrile

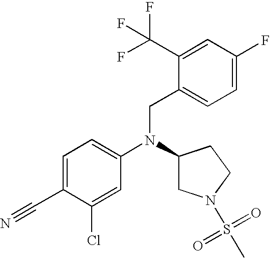

This compound was made according to general procedure of example 105 from 1-(bromomethyl)-4-fluoro-2-(trifluoromethyl)benzene. LC-MS (ES) m/e 476.2 [M+H]⁺.

### Example 128

### 2-chloro-4-{(cyclohexylmethyl)[(3S)-1-(methylsulfonyl)-3-pyrrolidinyl]amino}benzonitrile

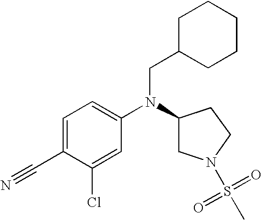

This compound was made according to general procedure of example 105 from (bromomethyl)cyclohexane. LC-MS (ES) m/e 396.4 [M+H]⁺.

### Example 129

### 2-chloro-4-{(3-methylbutyl)[(3S)-1-(methylsulfonyl)-3-pyrrolidinyl]amino}benzonitrile

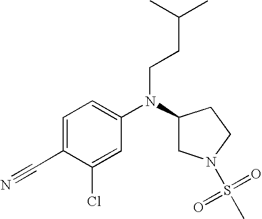

This compound was made according to general procedure of example 105 from 1-bromo-3-methylbutane. LC-MS (ES) m/e 370.2 [M+H]⁺.

### Example 130

### 2-chloro-4-{[(2-chloro-6-fluorophenyl)methyl][(3S)-1-(methylsulfonyl)-3-pyrrolidinyl]amino}benzonitrile

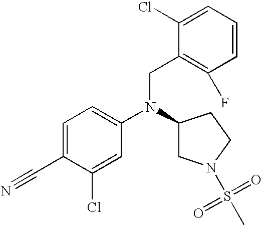

This compound was made according to general procedure of example 105 from 2-(bromomethyl)-1-chloro-3-fluorobenzene. LC-MS (ES) m/e 442.6 [M+H]⁺.

### Example 131

### 2-chloro-4-{[(2,3-difluorophenyl)methyl][(3S)-1-(methylsulfonyl)-3-pyrrolidinyl]amino}benzonitrile

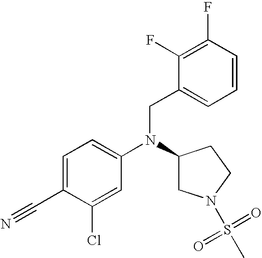

This compound was made according to general procedure of example 105 from 1-(bromomethyl)-2,3-difluorobenzene. LC-MS (ES) m/e 426.4 [M+H]⁺.

### Example 132

### 2-chloro-4-([(3S)-1-(methylsulfonyl)-3-pyrrolidinyl]{[2-methyl-5-(trifluoromethyl)phenyl]methyl}amino)benzonitrile

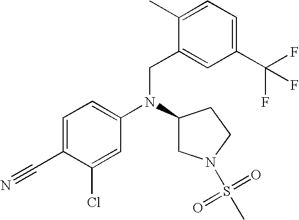

This compound was made according to general procedure of example 105 from 2-(bromomethyl)-1-methyl-4-(trifluoromethyl)benzene. LC-MS (ES) m/e 472.6 [M+H]⁺.

### Example 133

### 2-chloro-4-{[(2,5-difluorophenyl)methyl][(3S)-1-(methylsulfonyl)-3-pyrrolidinyl]amino}benzonitrile

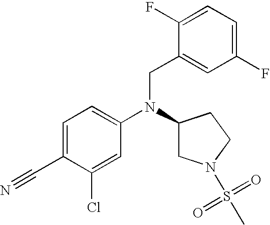

This compound was made according to general procedure of example 105 from 2-(bromomethyl)-1,4-difluorobenzene. LC-MS (ES) m/e 426.4 [M+H]⁺.

### Example 134

### 2-chloro-4-{[(3S)-1-(methylsulfonyl)-3-pyrrolidinyl][(2,4,5-trifluorophenyl)methyl]amino}benzonitrile

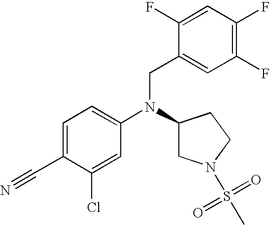

This compound was made according to general procedure of example 105 from 1-(bromomethyl)-2,4,5-trifluorobenzene. LC-MS (ES) m/e 444.6 [M+H]⁺.

### Example 135

### 2-chloro-4-{[(2,4-difluorophenyl)methyl][(3S)-1-(methylsulfonyl)-3-pyrrolidinyl]amino}benzonitrile

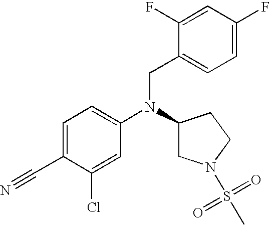

This compound was made according to general procedure of example 105 from 1-(bromomethyl)-2,4-difluorobenzene. LC-MS (ES) m/e 426.2 [M+H]⁺.

### Example 136

### 2-chloro-4-{{[5-chloro-2-(trifluoromethyl)phenyl]methyl}[(3S)-1-(methylsulfonyl)-3-pyrrolidinyl]amino}benzonitrile

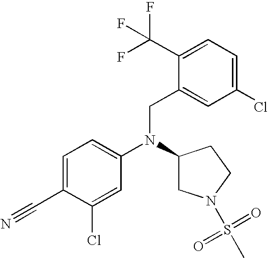

This compound was made according to general procedure of example 105 from 2-(bromomethyl)-4-chloro-1-(trifluoromethyl)benzene. LC-MS (ES) m/e 492.4 [M+H]⁺.

### Example 137

### 2-chloro-4-{{[5-fluoro-2-(trifluoromethyl)phenyl]methyl}[(3S)-1-methylsulfonyl)-3-pyrrolidinyl]amino}benzonitrile

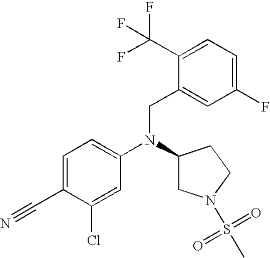

This compound was made according to general procedure of example 105 from 2-(bromomethyl)-4-fluoro-1-(trifluoromethyl)benzene. LC-MS (ES) m/e 476.4 [M+H]⁺.

### Example 138

### 2-chloro-4-{{[2-fluoro-6-(trifluoromethyl)phenyl]methyl}[(3S)-1-(methylsulfonyl)-3-pyrrolidinyl]amino}benzonitrile

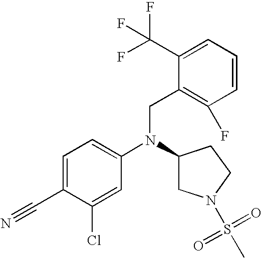

This compound was made according to general procedure of example 105 from 2-(bromomethyl)-1-fluoro-3-(trifluoromethyl)benzene. LC-MS (ES) m/e 476.4 [M+H]⁺.

### Example 139

### 2-chloro-4-([(3S)-1-(ethylsulfonyl)-3-pyrrolidinyl]{[2-(trifluoromethyl)phenyl]methyl}amino)benzonitrile

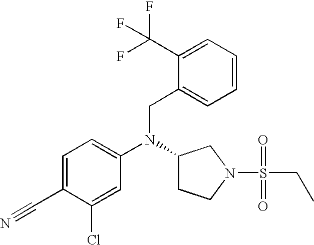

This compound was made according to general procedure of example 77 from 2-chloro-4-((3S)-3-pyrrolidinyl{[2-(trifluoromethyl)phenyl]methyl}amino)benzonitrile and ethanesulfonyl chloride.

LC-MS (ES) m/e 472.4 [M+H]⁺.

### Example 140

### 2-chloro-4-({(3S)-1-[(1-methylethyl)sulfonyl]-3-pyrrolidinyl}{[2-(trifluoromethyl)phenyl]methyl}amino)benzonitrile

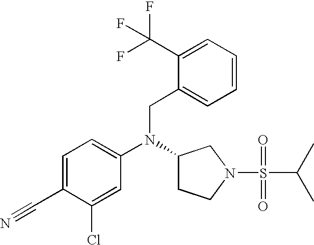

This compound was made according to general procedure of example 77 from 2-chloro-4-((3S)-3-pyrrolidinyl{[2-(trifluoromethyl)phenyl]methyl}amino)benzonitrile and 2-propanesulfonyl chloride. LC-MS (ES) m/e 486.4 [M+H]⁺.

### Example 141

### 2-chloro-4-([(2-chlorophenyl)methyl]{(3S)-1-[(1,3,5-trimethyl-1H-pyrazol-4-yl)sulfonyl]-3-pyrrolidinyl}amino)benzonitrile

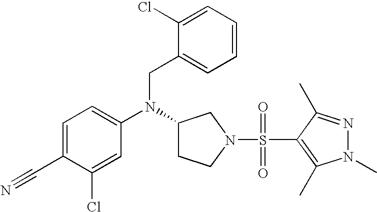

This compound was made according to general procedure of example 77 from 2-chloro-4-{[(2-chlorophenyl)methyl][(3S)-3-pyrrolidinyl]amino}benzonitrile and 1,3,5-trimethyl-1H-pyrazole-4-sulfonyl chloride. LC-MS (ES) m/e 518.6 [M+H]⁺.

### Example 142

### 2-chloro-4-{[(2-chlorophenyl)methyl][(3S)-1-phenylsulfonyl)-3-pyrrolidinyl]amino}benzonitrile

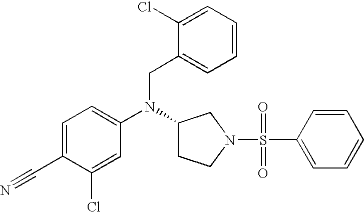

This compound was made according to general procedure of example 77 from 2-chloro-4-{[(2-chlorophenyl)methyl][(3S)-3-pyrrolidinyl]amino}benzonitrile and benzenesulfonyl chloride. LC-MS (ES) m/e 486.4 [M+H]⁺.

### Example 143

### 2-chloro-4-([(2-chlorophenyl)methyl]{(3S)-1-[(5-methyl-2-thienyl)sulfonyl]-3-pyrrolidinyl}amino)benzonitrile

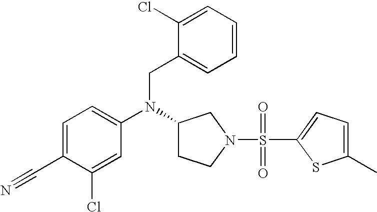

This compound was made according to general procedure of example 77 from 2-chloro-4-{[(2-chlorophenyl)methyl][(3S)-3-pyrrolidinyl]amino}benzonitrile and 5-methyl-2-thiophenesulfonyl

chloride. LC-MS (ES) m/e 506.4 [M+H]⁺.

### Example 144

### 2-chloro-4-([(2-chlorophenyl)methyl]{(3S)-1-[(4-chlorophenyl)sulfonyl]-3-pyrrolidinyl}amino)benzonitrile

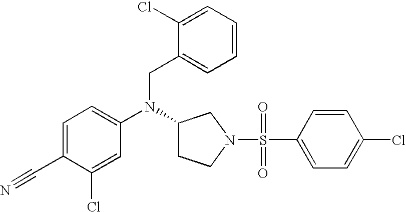

This compound was made according to general procedure of example 77 from 2-chloro-4-{[(2-chlorophenyl)methyl][(3S)-3-pyrrolidinyl]amino}benzonitrile and 4-chlorobenzenesulfonyl chloride. LC-MS (ES) m/e 520.4 [M+H]⁺.

### Example 145

### 2-chloro-4-([(2-chlorophenyl)methyl]{(3S)-1-[(2-chlorophenyl)sulfonyl]-3-pyrrolidinyl}amino)benzonitrile

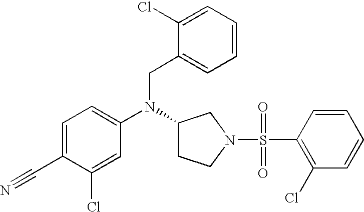

This compound was made according to general procedure of example 77 from 2-chloro-4-{[(2-chlorophenyl)methyl][(3S)-3-pyrrolidinyl]amino}benzonitrile and 2-chlorobenzenesulfonyl chloride. LC-MS (ES) m/e 520.2 [M+H]⁺.

### Example 146

### 2-chloro-4-[[(2-chlorophenyl)methyl]((3S)-1-{[5-(2-pyridinyl)-2-thienyl]sulfonyl}-3-pyrrolidinyl)amino]benzonitrile

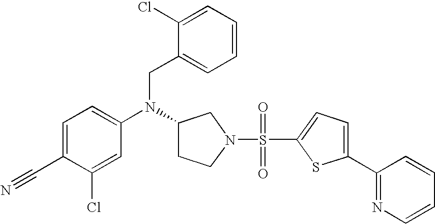

This compound was made according to general procedure of example 77 from 2-chloro-4-{[(2-chlorophenyl)methyl][(3S)-3-pyrrolidinyl]amino}benzonitrile and 5-(2-pyridinyl)-2-thiophenesulfonyl chloride. LC-MS (ES) m/e 569.4 [M+H]⁺.

### Example 147

### 2-chloro-4-([(2-chlorophenyl)methyl]{(3S)-1-[(1-methylethyl)sulfonyl]-3-pyrrolidinyl}amino)benzonitrile

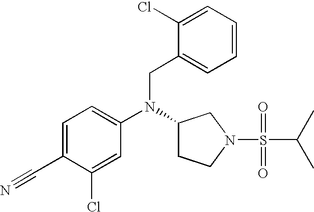

This compound was made according to general procedure of example 77 from 2-chloro-4-{[(2-chlorophenyl)methyl][(3S)-3-pyrrolidinyl]amino}benzonitrile and 2-propanesulfonyl chloride.

LC-MS (ES) m/e 452.4 [M+H]⁺.

### Example 148

### 2-chloro-4-([(2-chlorophenyl)methyl]{(3S)-1-[(4,5-dichloro-2-thienyl)sulfonyl]-3-pyrrolidinyl}amino)benzonitrile

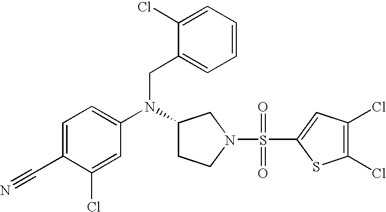

This compound was made according to general procedure of example 77 from 2-chloro-4-{[(2-chlorophenyl)methyl][(3S)-3-pyrrolidinyl]amino}benzonitrile and 4,5-dichloro-2-thiophenesulfonyl chloride. LC-MS (ES) m/e 560.0 [M+H]⁺.

### Example 149

### 2-chloro-4-[[(2-chlorophenyl)methyl]((3S)-1-{[4-(methyloxy)phenyl]sulfonyl}-3-pyrrolidinyl)amino]benzonitrile

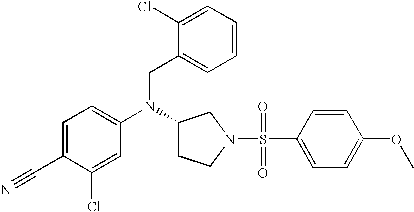

This compound was made according to general procedure of example 77 from 2-chloro-4-{[(2-chlorophenyl)methyl][(3S)-3-pyrrolidinyl]amino}benzonitrile and 4-(methyloxy)benzenesulfonyl chloride. LC-MS (ES) m/e 516.2 [M+H]⁺.

### Example 150

### 4-{{(3S)-1-[(5-bromo-2-thienyl)sulfonyl]-3-pyrrolidinyl}[2-chlorophenyl)methyl]amino}-2-chlorobenzonitrile

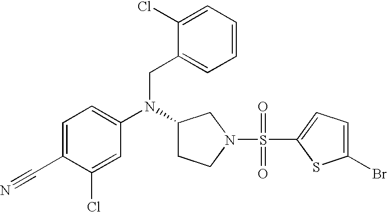

This compound was made according to general procedure of example 77 from 2-chloro-4-{[(2-chlorophenyl)methyl][(3S)-3-pyrrolidinyl]amino}benzonitrile and 5-bromo-2-thiophenesulfonyl chloride. LC-MS (ES) m/e 570.2 [M]⁺.

### Example 151

### 2-chloro-4-[[(2-chlorophenyl)methyl]((3S)-1-{[3-(methyloxy)phenyl]sulfonyl}-3-pyrrolidinyl)amino]benzonitrile

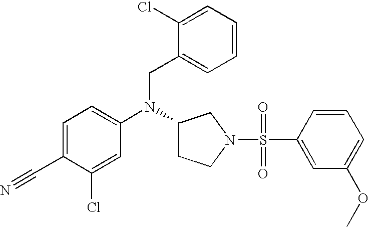

This compound was made according to general procedure of example 77 from 2-chloro-4-{[(2-chlorophenyl)methyl][(3S)-3-pyrrolidinyl]amino}benzonitrile and 3-(methyloxy)benzenesulfonyl chloride. LC-MS (ES) m/e 516.4 [M+H]⁺.

### Example 152

### 2-chloro-4-([(2-chlorophenyl)methyl]{(3S)-1-[(trifluoromethyl)sulfonyl]-3-pyrrolidinyl}amino)benzonitrile

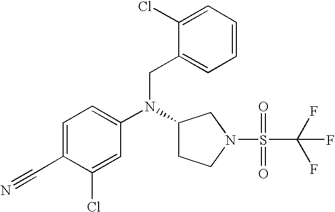

This compound was made according to general procedure of example 77 from 2-chloro-4-{[(2-chlorophenyl)methyl][(3S)-3-pyrrolidinyl]amino}benzonitrile and trifluoromethanesulfonyl chloride. LC-MS (ES) m/e 478.2 [M+H]⁺.

### Example 153

### 4-{{(3S)-1-[(5-bromo-6-chloro-3-pyridinyl)sulfonyl]-3-pyrrolidinyl}[(2-chlorophenyl)methyl]amino}-2-chlorobenzonitrile

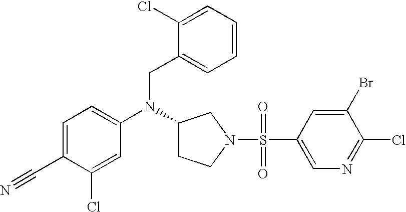

This compound was made according to general procedure of example 77 from 2-chloro-4-{[(2-chlorophenyl)methyl][(3S)-3-pyrrolidinyl]amino}benzonitrile and 5-bromo-6-chloro-3-pyridinesulfonyl chloride. LC-MS (ES) m/e 599.2 [M]⁺.

### Example 154

### 2-chloro-4-{[(2-chlorophenyl)methyl][(3S)-1-(ethylsulfonyl)-3-pyrrolidinyl]amino}benzonitrile

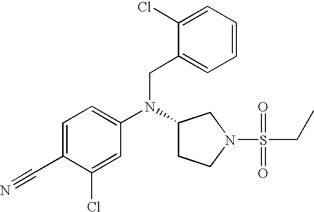

This compound was made according to general procedure of example 77 from 2-chloro-4-{[(2-chlorophenyl)methyl][(3S)-3-pyrrolidinyl]amino}benzonitrile and ethanesulfonyl chloride. LC-MS (ES) m/e 438.2 [M+H]⁺.

### Example 155

### 2-chloro-4-([(2-chlorophenyl)methyl]{(3S)-1-[(1,2-dimethyl-1H-imidazol-4-yl)sulfonyl]-3-pyrrolidinyl}amino)benzonitrile

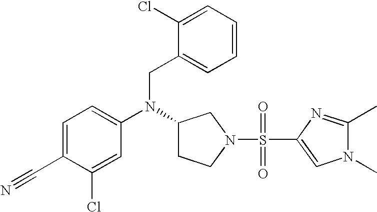

This compound was made according to general procedure of example 77 from 2-chloro-4-{[(2-chlorophenyl)methyl][(3S)-3-pyrrolidinyl]amino}benzonitrile and 1,2-dimethyl-1H-imidazole-4-sulfonyl chloride. LC-MS (ES) m/e 504.2 [M+H]⁺.

### Example 156

### 2-chloro-4-([(2-chlorophenyl)methyl]{(3S)-1-[(2-cyanophenyl)sulfonyl]-3-pyrrolidinyl}amino)benzonitrile

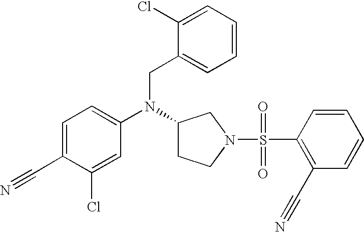

This compound was made according to general procedure of example 77 from 2-chloro-4-{[(2-chlorophenyl)methyl][(3S)-3-pyrrolidinyl]amino}benzonitrile and 2-cyanobenzenesulfonyl chloride. LC-MS (ES) m/e 513.6 [M+H]⁺.

### Example 157

### 2-chloro-4-{[(2-chlorophenyl)methyl][(3S)-1-(cyclopropylsulfonyl)-3-pyrrolidinyl]amino}benzonitrile

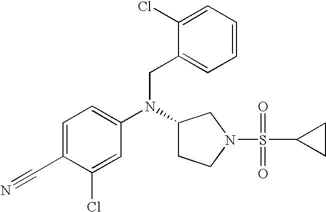

This compound was made according to general procedure of example 77 from 2-chloro-4-{[(2-chlorophenyl)methyl][(3S)-3-pyrrolidinyl]amino}benzonitrile and cyclopropanesulfonyl chloride.

LC-MS (ES) m/e 450.4 [M+H]⁺.

### Example 158

### 2-chloro-4-{[(2-chlorophenyl)methyl][(3S)-1-(2-thienylsulfonyl)-3-pyrrolidinyl]amino}benzonitrile

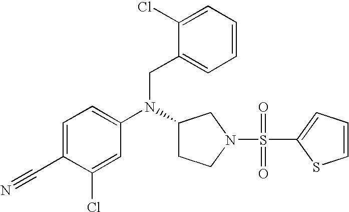

This compound was made according to general procedure of example 77 from 2-chloro-4-{[(2-chlorophenyl)methyl][(3S)-3-pyrrolidinyl]amino}benzonitrile and 2-thiophenesulfonyl chloride.

LC-MS (ES) m/e 492.4 [M+H]⁺.

### Example 159

### 2-chloro-4-([(2-chlorophenyl)methyl]{(3S)-1-[(3-chlorophenyl)sulfonyl]-3-pyrrolidinyl}amino)benzonitrile

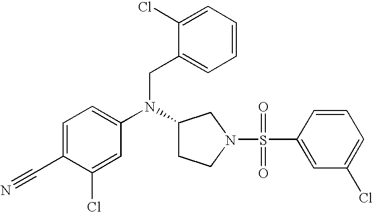

This compound was made according to general procedure of example 77 from 2-chloro-4-{[(2-chlorophenyl)methyl][(3S)-3-pyrrolidinyl]amino}benzonitrile and 3-chlorobenzenesulfonyl chloride. LC-MS (ES) m/e 520.2 [M+H]⁺.

### Example 160

### 2-chloro-4-([(2-chlorophenyl)methyl]{(3S)-1-[(2-fluorophenyl)sulfonyl]-3-pyrrolidinyl}amino)benzonitrile

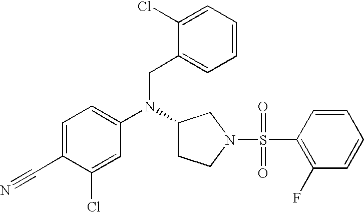

This compound was made according to general procedure of example 77 from 2-chloro-4-{[(2-chlorophenyl)methyl][(3S)-3-pyrrolidinyl]amino}benzonitrile and 2-fluorobenzenesulfonyl chloride. LC-MS (ES) m/e 504.2 [M+H]⁺.

### Example 161

### 2-chloro-4-([(2-chlorophenyl)methyl]{(3S)-1-[(4-fluorophenyl)sulfonyl]-3-pyrrolidinyl}amino)benzonitrile

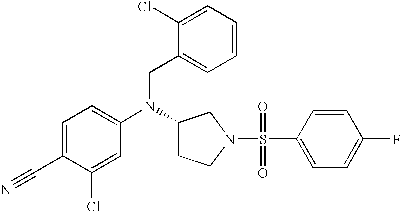

This compound was made according to general procedure of example 77 from 2-chloro-4-{[(2-chlorophenyl)methyl][(3S)-3-pyrrolidinyl]amino}benzonitrile and 4-fluorobenzenesulfonyl chloride. LC-MS (ES) m/e 504.2 [M+H]⁺.

### Example 162

### 2-chloro-4-([(2-chlorophenyl)methyl]{(3S)1-[(3-fluorophenyl)sulfonyl]-3-pyrrolidinyl}amino)benzonitrile

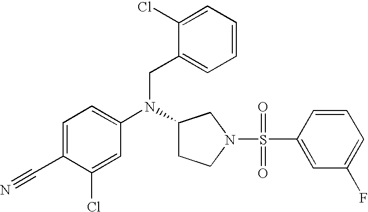

This compound was made according to general procedure of example 77 from 2-chloro-4-{[(2-chlorophenyl)methyl][(3S)-3-pyrrolidinyl]amino}benzonitrile and 3-fluorobenzenesulfonyl chloride. LC-MS (ES) m/e 504.2 [M+H]⁺.

### Example 163

### 2-chloro-4-([(2-chlorophenyl)methyl]{(3S)-1-[(phenylmethyl)sulfonyl]-3-pyrrolidinyl}amino)benzonitrile

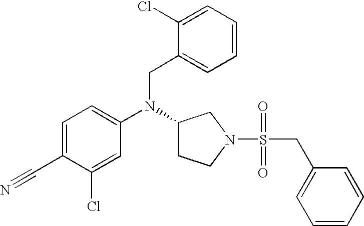

This compound was made according to general procedure of example 77 from 2-chloro-4-{[(2-chlorophenyl)methyl][(3S)-3-pyrrolidinyl]amino}benzonitrile and phenylmethanesulfonyl chloride.

LC-MS (ES) m/e 500.2 [M+H]⁺.

### Example 164

### 2-chloro-4-{{(3S)-1-[(4-chlorophenyl)sulfonyl]-3-pyrrolidinyl}[2-methylphenyl)methyl]amino}benzonitrile

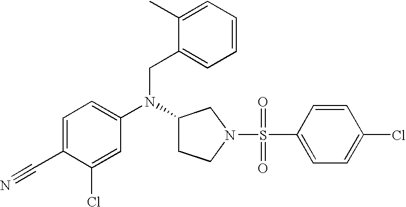

This compound was made according to general procedure of example 77 from 4-chlorobenzenesulfonyl chloride. LC-MS (ES) m/e 500.6 [M+H]⁺.

### Example 165

### 2-chloro-4-{{(3S)-1[(3-fluorophenyl)sulfonyl]-3-pyrrolidinyl}[(2-methylphenyl)methyl]amino}benzonitrile

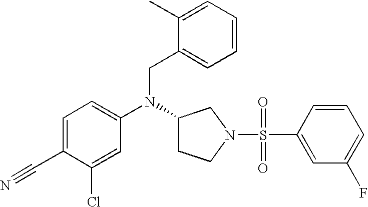

This compound was made according to general procedure of example 77 from 3-fluorobenzenesulfonyl chloride. LC-MS (ES) m/e 484.4 [M+H]⁺.

### Example 166

### 2-chloro-4-([(2-methylphenyl)methyl]{(3S)-1-[(5-methyl-2-thienyl)sulfonyl]-3-pyrrolidinyl}amino)benzonitrile

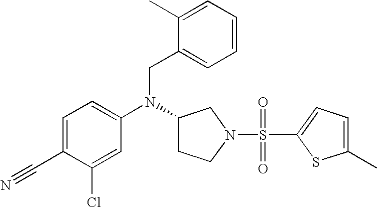

This compound was made according to general procedure of example 77 from 5-methyl-2-thiophenesulfonyl chloride. LC-MS (ES) m/e 486.4 [M+H]⁺.

### Example 167

### 2-chloro-4-{((3S)-1-{[4-(methyloxy)phenyl]sulfonyl}-3-pyrrolidinyl)[(2-methylphenyl)methyl]amino}benzonitrile

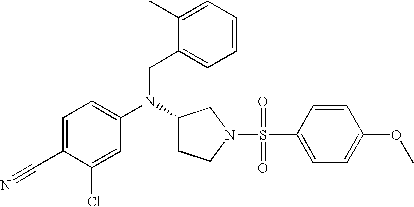

This compound was made according to general procedure of example 77 from 4-(methyloxy)benzenesulfonyl chloride. LC-MS (ES) m/e 496.4 [M+H]⁺.

### Example 168

### 2-chloro-4-([(2-methylphenyl)methyl]{(3S)-1-[(phenylmethyl)sulfonyl]-3-pyrrolidinyl}amino)benzonitrile

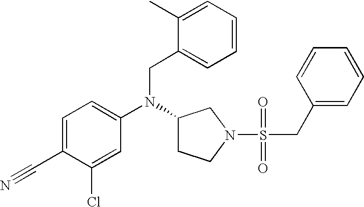

This compound was made according to general procedure of example 77 from phenylmethanesulfonyl chloride. LC-MS (ES) m/e 480.6 [M+H]⁺.

### Example 169

### 2-chloro-4-{{(3S)-1-[(1-methylethyl)sulfonyl]-3-pyrrolidinyl}[(2-methylphenyl)methyl]amino}benzonitrile

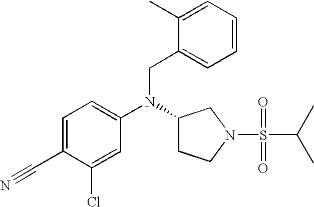

This compound was made according to general procedure of example 77 from 2-propanesulfonyl chloride. LC-MS (ES) m/e 432.6 [M+H]⁺.

### Example 170

### 2-chloro-4-{[(3S)-1-(ethylsulfonyl)-3-pyrrolidinyl][(2-methylphenyl)methyl]amino}benzonitrile

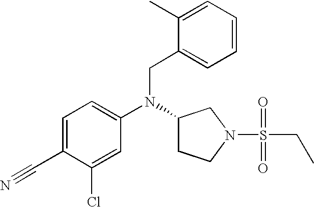

This compound was made according to general procedure of example 77 from ethanesulfonyl chloride. LC-MS (ES) m/e 418.6 [M+H]⁺.

### Example 171

### 2-chloro-4-{{(3S)-1-[(2-chlorophenyl)sulfonyl]-3-pyrrolidinyl}[(2-methylphenyl)methyl]amino}benzonitrile

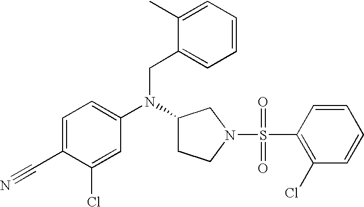

This compound was made according to general procedure of example 77 from 2-chlorobenzenesulfonyl chloride. LC-MS (ES) m/e 500.4 [M+H]⁺.

### Example 172

### 2-chloro-4-{{(3S)-1-[(4-fluorophenyl)sulfonyl]-3-pyrrolidinyl}[(2-methylphenyl)methyl]amino}benzonitrile

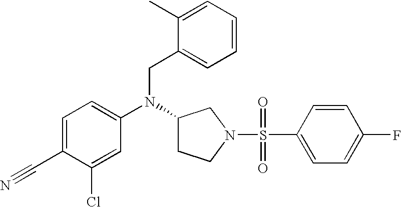

This compound was made according to general procedure of example 77 from 4-fluorobenzenesulfonyl chloride. LC-MS (ES) m/e 484.2 [M+H]⁺.

### Example 173

### 2-chloro-4-{((3S)-1-{[3-(methyloxy)phenyl]sulfonyl}-3-pyrrolidinyl)[(2-methylphenyl)methyl]amino}benzonitrile

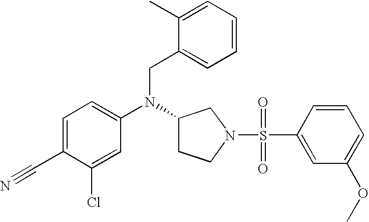

This compound was made according to general procedure of example 77 from 3-(methyloxy)benzenesulfonyl chloride. LC-MS (ES) m/e 496.4 [M+H]⁺.

### Example 174

### 2-chloro-4-{{(3S)-1[(2-fluorophenyl)sulfonyl]-3-pyrrolidinyl}[2-methylphenyl)methyl]amino}benzonitrile

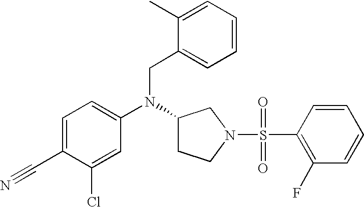

This compound was made according to general procedure of example 77 from 2-fluorobenzenesulfonyl chloride. LC-MS (ES) m/e 484.4 [M+H]⁺.

### Example 175

### 2-chloro-4-{[(2-methylphenyl)methyl][(3S)-1-(2-thienylsulfonyl)-3-pyrrolidinyl]amino}benzonitrile

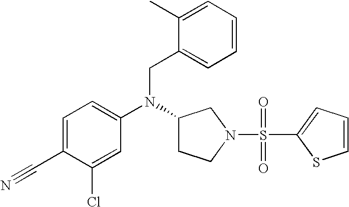

This compound was made according to general procedure of example 77 from 2-thiophenesulfonyl chloride. LC-MS (ES) m/e 472.4 [M+H]⁺.

### Example 176

### 2-chloro-4-{{(3S)-1-[(3-chlorophenyl)sulfonyl]-3-pyrrolidinyl}[(2-methylphenyl)methyl]amino}benzonitrile

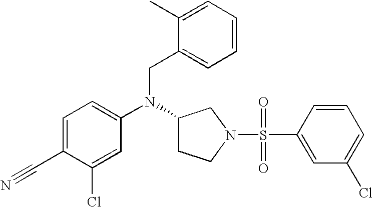

This compound was made according to general procedure of example 77 from 3-chlorobenzenesulfonyl chloride. LC-MS (ES) m/e 500.4 [M+H]⁺.

### Example 177

### 2-chloro-4-[[(2-chlorophenyl)methyl]((3S)-1-{[(3,5-dichlorophenyl)methyl]sulfonyl}-3-pyrrolidinyl)amino]benzonitrile

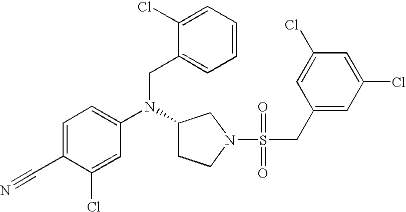

This compound was made according to general procedure of example 77 from 2-chloro-4-{[(2-chlorophenyl)methyl][(3S)-3-pyrrolidinyl]amino}benzonitrile and (3,5-dichlorophenyl)methanesulfonyl chloride. LC-MS (ES) m/e 568.4 [M+H]⁺.

### Example 178

### N-{4-[((3S)-3-{(3-chloro-4-cyanophenyl)[(2-chlorophenyl)methyl]amino}-1-pyrrolidinyl)sulfonyl]-3-methylphenyl}acetamide

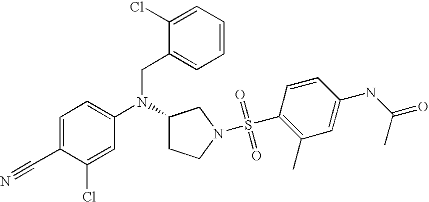

This compound was made according to general procedure of example 77 from 2-chloro-4-{[(2-chlorophenyl)methyl][(3S)-3-pyrrolidinyl]amino}benzonitrile and 4-(acetylamino)-2-methylbenzenesulfonyl chloride. LC-MS (ES) m/e 557.0 [M+H]⁺.

### Example 179

### 2-chloro-4-[[(2-chlorophenyl)methyl]((3S)-1-{[(4-methylphenyl)methyl]sulfonyl}-3-pyrrolidinyl)amino]benzonitrile

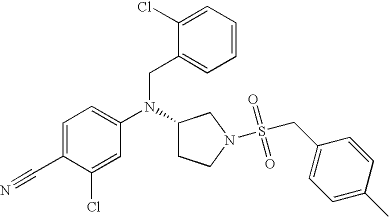

This compound was made according to general procedure of example 77 from 2-chloro-4-{[(2-chlorophenyl)methyl][(3S)-3-pyrrolidinyl]amino}benzonitrile and (4-methylphenyl)methanesulfonyl chloride. LC-MS (ES) m/e 514.6 [M+H]⁺.

### Example 180

### 2-chloro-4-{[(2-chlorophenyl)methyl][(3S)-1-({[3-(trifluoromethyl)phenyl]methyl}sulfonyl)-3-pyrrolidinyl]amino}benzonitrile

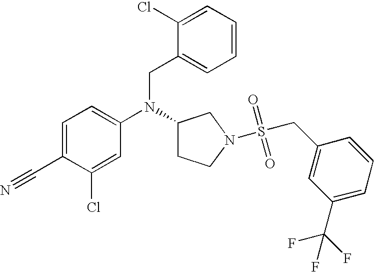

This compound was made according to general procedure of example 77 from 2-chloro-4-{[(2-chlorophenyl)methyl][(3S)-3-pyrrolidinyl]amino}benzonitrile and [3-(trifluoromethyl)phenyl]methanesulfonyl chloride. LC-MS (ES) m/e 568.4 [M+H]⁺.

### Example 181

### methyl 3-[((3S)-3-{(3-chloro-4-cyanophenyl)[(2-chlorophenyl)methyl]amino}-1-pyrrolidinyl)sulfonyl]-2-thiophenecarboxylate

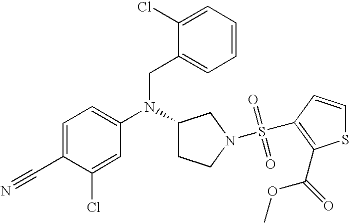

This compound was made according to general procedure of example 77 from 2-chloro-4-{[(2-chlorophenyl)methyl][(3S)-3-pyrrolidinyl]amino}benzonitrile and methyl 3-(chlorosulfonyl)-2-thiophenecarboxylate. LC-MS (ES) m/e 550.4 [M+H]⁺.

### Example 182

### 2-chloro-4-[[(2-chlorophenyl)methyl]((3S)-1-{[(4-chlorophenyl)methyl]sulfonyl}-3-pyrrolidinyl)amino]benzonitrile

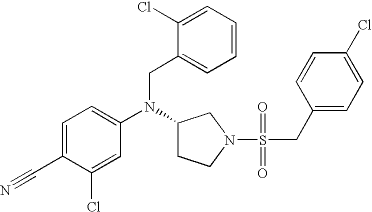

This compound was made according to general procedure of example 77 from 2-chloro-4-{[(2-chlorophenyl)methyl][(3S)-3-pyrrolidinyl]amino}benzonitrile and (4-chlorophenyl)methanesulfonyl chloride. LC-MS (ES) m/e 534.2 [M+H]⁺.

### Example 183

### 2-chloro-4-{((3S)-1-{[2-(4-chlorophenyl)ethyl]sulfonyl}-3-pyrrolidinyl)[(2-chlorophenyl)methyl]amino}benzonitrile

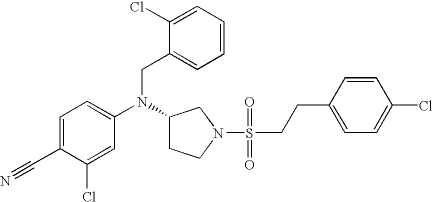

This compound was made according to general procedure of example 77 from 2-chloro-4-{[(2-chlorophenyl)methyl][(3S)-3-pyrrolidinyl]amino}benzonitrile and 2-(4-chlorophenyl)ethanesulfonyl chloride. LC-MS (ES) m/e 548.4 [M+H]⁺.

### Example 184

### 4-{[(3S)-1-(butylsulfonyl)-3-pyrrolidinyl][(2-methylphenyl)methyl]amino}-2-chlorobenzonitrile

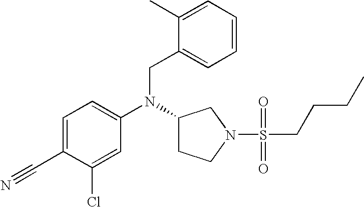

This compound was made according to general procedure of example 77 from 1-butanesulfonyl chloride. LC-MS (ES) m/e 446.2 [M+H]⁺.

### Example 185

### 2-chloro-4-{[(2-methylphenyl)methyl][(3S)-1-(pentylsulfonyl)-3-pyrrolidinyl]amino}benzonitrile

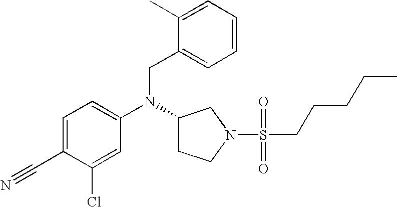

This compound was made according to general procedure of example 77 from 1-pentanesulfonyl chloride. LC-MS (ES) m/e 460.4 [M+H]⁺.

### Example 186

### 2-chloro-4-([(2-methylphenyl)methyl]{(3S)-1-[(2,2,2-trifluoroethyl)sulfonyl]-3-pyrrolidinyl}amino)benzonitrile

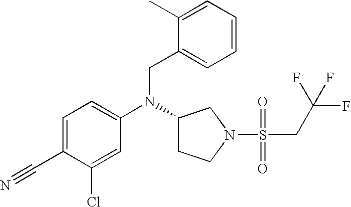

This compound was made according to general procedure of example 77 from 2,2,2-trifluoroethanesulfonyl chloride. LC-MS (ES) m/e 472.4 [M+H]⁺.

### Example 187

### 2-chloro-4-{[(2-methylphenyl)methyl][(3S)-1-(propylsulfonyl)-3-pyrrolidinyl]amino}benzonitrile

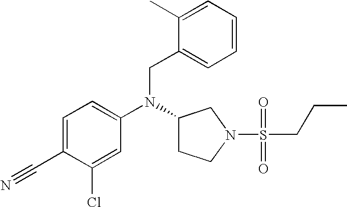

This compound was made according to general procedure of example 77 from 1-propanesulfonyl chloride. LC-MS (ES) m/e 432.6 [M+H]⁺.

### Example 188

### 2-chloro-4-{[(3S)-1-(ethylsulfonyl)-3-pyrrolidinyl][(2-fluorophenyl)methyl]amino}benzonitrile

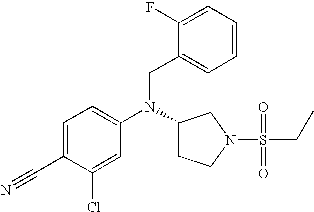

This compound was made according to general procedure of example 77 from 2-chloro-4-{[(2-fluorophenyl)methyl][(3S)-3-pyrrolidinyl]amino}benzonitrile and ethanesulfonyl chloride. LC-MS (ES) m/e 422.2 [M+H]⁺.

### Example 189

### 2-chloro-4-([(2-fluorophenyl)methyl]{(3S)-1-[(1-methylethyl)sulfonyl]-3-pyrrolidinyl}amino)benzonitrile

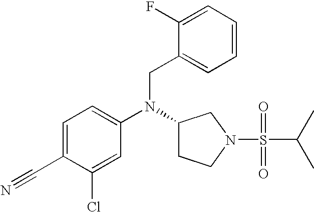

This compound was made according to general procedure of example 77 from 2-chloro-4-{[(2-fluorophenyl)methyl][(3S)-3-pyrrolidinyl]amino}benzonitrile and 2-propanesulfonyl chloride. LC-MS (ES) m/e 436.1 [M+H]⁺.

### Example 190

### 2-chloro-4-([2,3-difluorophenyl)methyl]{(3S)-1-[(1-methylethyl)sulfonyl]-3-pyrrolidinyl}amino)benzonitrile

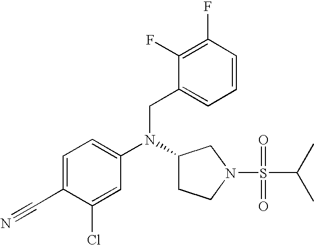

### a) 2-chloro-4-{[(2,3-difluorophenyl)methyl][(3S)-3-pyrrolidinyl]amino}benzonitrile

This compound was made according to general procedures of example 1 (part b and c) from 1-(bromomethyl)-2,3-difluorobenzene. LC-MS (ES) m/e 348.4 [M+H]⁺.

### b) 2-chloro-4-([(2,3-difluorophenyl)methyl]{(3S)-1-[(1-methylethyl)sulfonyl]-3-pyrrolidinyl}amino)benzonitrile

This compound was made according to general procedure of example 77 from 2-chloro-4-{[(2,3-difluorophenyl)methyl][(3S)-3-pyrrolidinyl]amino}benzonitrile and 2-propanesulfonyl chloride.

LC-MS (ES) m/e 454.2 [M+H]⁺.

### Example 191

### 2-chloro-4-{[(2,3-difluorophenyl)methyl][(3S)-1-(ethylsulfonyl)-3-pyrrolidinyl]amino}benzonitrile

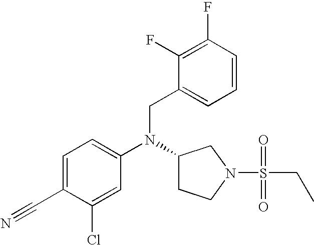

This compound was made according to general procedure of example 190 from ethanesulfonyl chloride. LC-MS (ES) m/e 440.4 [M+H]⁺.

### Example 192

### 2-chloro-4-{[(2-chloro-5-fluorophenyl)methyl][(3S)-1-(ethylsulfonyl)-3-pyrrolidinyl]amino}benzonitrile

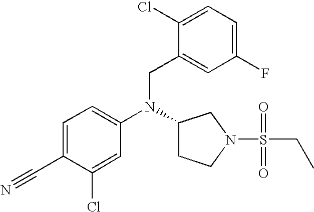

### a) 2-chloro-4-{[(2-chloro-5-fluorophenyl)methyl][(3S)-3-pyrrolidinyl]amino}benzonitrile

This compound was made according to general procedures of example 1 (part b and c) from 2-(bromomethyl)-1-chloro-4-fluorobenzene. LC-MS (ES) m/e 364.4 [M+H]⁺.

### b) 2-chloro-4-{[(2-chloro-5-fluorophenyl)methyl][(3S)-1-(ethylsulfonyl)-3-pyrrolidinyl]amino}benzonitrile

This compound was made according to general procedure of example 77 from 2-chloro-4-{[(2-chloro-5-fluorophenyl)methyl][(3S)-3-pyrrolidinyl]amino}benzonitrile and ethanesulfonyl chloride.

LC-MS (ES) m/e 456.4 [M+H]⁺.

### Example 193

### 2-chloro-4-([(2-chloro-5-fluorophenyl)methyl]{(3S)-1-[(1-methylethyl)sulfonyl]-3-pyrrolidinyl}amino)benzonitrile

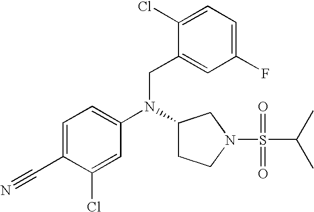

This compound was made according to general procedure of example 192 from 2-propanesulfonyl chloride. LC-MS (ES) m/e 470.2 [M+H]⁺.

### Example 194

### 2-chloro-4-([(5-fluoro-2-methylphenyl)methyl]{(3S)-1-[(1-methylethyl)sulfonyl]-3-pyrrolidinyl}amino)benzonitrile

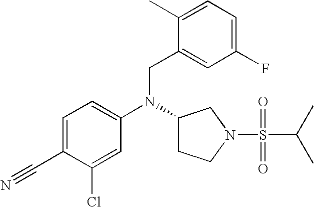

### a) 2-chloro-4-{[(5-fluoro-2-methylphenyl)methyl][(3S)-3-pyrrolidinyl]amino}benzonitrile

This compound was made according to general procedures of example 1 (part b and c) from 2-(bromomethyl)-4-fluoro-1-methylbenzene. LC-MS (ES) m/e 344.4 [M+H]⁺.

### b) 2-chloro-4-([(5-fluoro-2-methylphenyl)methyl]{(3S)-1-[(1-methylethyl)sulfonyl]-3-pyrrolidinyl}amino)benzonitrile

This compound was made according to general procedure of example 77 from 2-chloro-4-{[(5-fluoro-2-methylphenyl)methyl][(3S)-3-pyrrolidinyl]amino}benzonitrile and 2-propanesulfonyl

chloride. LC-MS (ES) m/e 450.4 [M+H]⁺.

### Example 195

### 2-chloro-4-{[(3S)-1-(ethylsulfonyl)-3-pyrrolidinyl][(5-fluoro-2-methylphenyl)methyl]amino}benzonitrile

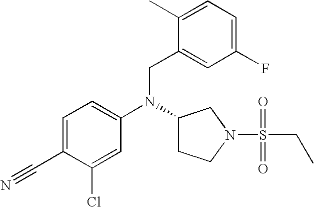

This compound was made according to general procedure of example 194 from ethanesulfonyl chloride. LC-MS (ES) m/e 436.4 [M+H]⁺.

### Example 196

### 2-chloro-4-{[(2,5-dichlorophenyl)methyl][(3S)-1-(ethylsulfonyl)-3-pyrrolidinyl]amino}benzonitrile

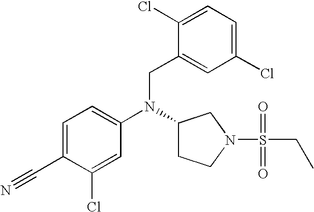

### a) 2-chloro-4-{[(2,5-dichlorophenyl)methyl][(3S)-3-pyrrolidinyl]amino}benzonitrile

This compound was made according to general procedures of example 1 (part b and c) from 2-(bromomethyl)-1,4-dichlorobenzene. LC-MS (ES) m/e 380.4 [M+H]⁺.

### b) 2-chloro-4-{[(2,5-dichlorophenyl)methyl][(3S)-1-(ethylsulfonyl)-3-pyrrolidinyl]amino}benzonitrile

This compound was made according to general procedure of example 77 from 2-chloro-4-{[(2,5-dichlorophenyl)methyl][(3S)-3-pyrrolidinyl]amino}benzonitrile and ethanesulfonyl chloride. LC-MS (ES) m/e 472.2 [M+H]⁺.

### Example 197

### 2-chloro-4-([(2,5-dichlorophenyl)methyl]{(3S)-1-[(1-methylethyl)sulfonyl]-3-pyrrolidinyl}amino)benzonitrile

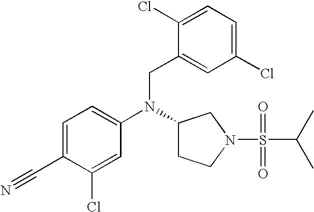

This compound was made according to general procedure of example 196 from 2-propanesulfonyl chloride. LC-MS (ES) m/e 488.0 [M+H]⁺.

### Example 198

### 4-([(2-bromophenyl)methyl]{(3S)-1-[(1-methylethyl)sulfonyl]-3-pyrrolidinyl}amino)-2-chlorobenzonitrile

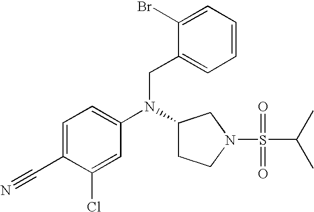

### a) 4-{[(2-bromophenyl)methyl][(3S)-3-pyrrolidinyl]amino}-2-chlorobenzonitrile

This compound was made according to general procedures of example 1 (part b and c) from 1-bromo-2-(bromomethyl)benzene. LC-MS (ES) m/e 390.2 [M+H]⁺.

### b) 4-([(2-bromophenyl)methyl]{(3S)-1-[(1-methylethyl)sulfonyl]-3-pyrrolidinyl}amino)-2-chlorobenzonitrile

This compound was made according to general procedure of example 77 from 4-{[(2-bromophenyl)methyl][(3S)-3-pyrrolidinyl]amino}-2-chlorobenzonitrile and 2-propanesulfonyl chloride. LC-MS (ES) m/e 496.4 [M]⁺.

### Example 199

### 4-{[(2-bromophenyl)methyl][(3S)-1-(ethylsulfonyl)-3-pyrrolidinyl]amino}-2-chlorobenzonitrile

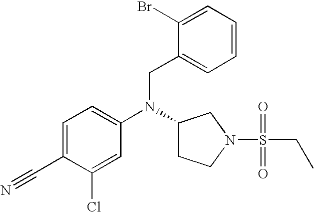

This compound was made according to general procedure of example 198 from ethanesulfonyl chloride. LC-MS (ES) m/e 482.0 [M]⁺.

### Example 200

### 2-chloro-4-([(2,5-difluorophenyl)methyl]{(3S)-1-[(1-methylethyl)sulfonyl]-3-pyrrolidinyl}amino)benzonitrile

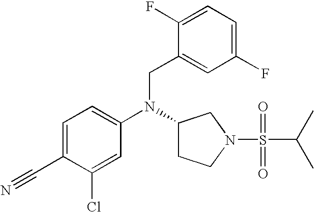

### a) 2-chloro-4-{[(2,5-difluorophenyl)methyl][(3S)-3-pyrrolidinyl]amino}benzonitrile

This compound was made according to general procedures of example 1 (part b and c) from 2-(bromomethyl)-1,4-difluorobenzene. LC-MS (ES) m/e 348.2 [M+H]⁺.

### b) 2-chloro-4-([(2,5-difluorophenyl)methyl]{(3S)-1-[(1-methylethyl)sulfonyl]-3-pyrrolidinyl}amino)benzonitrile

This compound was made according to general procedure of example 77 from 2-chloro-4-{[(2,5-difluorophenyl)methyl][(3S)-3-pyrrolidinyl]amino}benzonitrile and 2-propanesulfonyl chloride.

LC-MS (ES) m/e 454.4 [M+H]⁺.

### Example 201

### 2-chloro-4-{[(2,5-difluorophenyl)methyl][(3S)-1-(ethylsulfonyl)-3-pyrrolidinyl]amino}benzonitrile

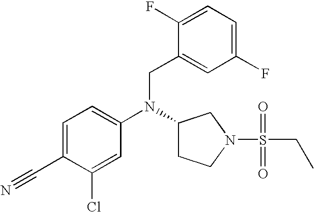

This compound was made according to general procedure of example 200 from ethanesulfonyl chloride. LC-MS (ES) m/e 440.4 [M+H]⁺.

### Example 202

### methyl 4-chloro-3-({(3-chloro-4-cyanophenyl)[(3S)-1-(methylsulfonyl)-3-pyrrolidinyl]amino}methyl)benzoate

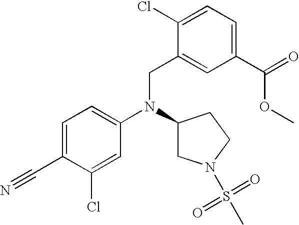

### a) Methyl 3-(bromomethyl)-4-chlorobenzoate

To a solution of methyl 4-chloro-3-methylbenzoate (2.22 g, 12 mmol) in CCl₄ (16 mL) was added NBS (2.35 g, 13.2 mmol) and AIBN (0.031 g, 0.19 mmol) and the reaction was refluxed for 3 h. After cooled down, the reaction was filtered and the filtrate was washed with H₂O, dried over Na₂SO₄, and concentrated. The solid residue was recrystallized with EtOAc and hexane (1:4) to yield the titled product (0.95 g, 30%) as clear crystals. ¹H-NMR (CDCl₃) δ: 3.949 (s, 3H), 4.628 (s, 2H,), 7.494 (d, 1H, J=8.4 Hz), 7.942 (d, 1H, J=8.4 Hz), 8.143 (s, 1H).

### b) Methyl 4-chloro-3-({(3-chloro-4-cyanophenyl)[(3S)-1-(methylsulfonyl)-3-pyrrolidinyl]amino}methyl)benzoate

This compound was made according to general procedure of example 105 from methyl 3-(bromomethyl)-4-chlorobenzoate with the following exception: after working up the reaction mixture was acidified with 1N HCl, extracted with EtOAc, and concentrated. MeOH (10 mL) and concentrated H₂SO₄ (2 drops) were added to the residue and the reaction was stirred at 65° C. until the esterification completed. Solvent was removed; saturated NaHCO₃ was added and the reaction was extracted with CH₂Cl₂. The organic layer was dried over Na₂SO₄, concentrated, and purified via column chromatography (cluted with 5% EtOAc in hexane grading to 55% EtOAc in hexane) to yield the titled compound as a white solid. LC-MS (ES) m/e 483.0 [M+H]⁺.

### Example 203

### 4-chloro-3-({(3-chloro-4-cyanophenyl)[(3S)-1-(methylsulfonyl)-3-pyrrolidinyl]amino}methyl)benzoic acid

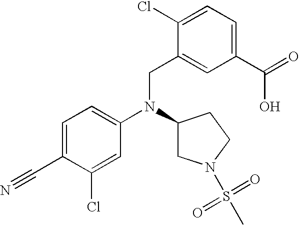

A mixture of methyl 4-chloro-3-({(3-chloro-4-cyanophenyl)[(3S)-1-(methylsulfonyl)-3-pyrrolidinyl]amino}methyl)benzoate (0.038 g, 0.078 mmol), 2N NaOH (0.5 mL) and THF (3 mL) was stirred at RT for 3 days. Solvent was removed and the reaction was acidified with 1N HCl, extracted with EtOAc, and concentrated. The residue was purified by HPLC to give the product (0.02 g, 54%) as a white solid. LC-MS (ES) m/e 468.4 [M+H]⁺.

### Example 204

### 2-chloro-4-{[(2,5-dichlorophenyl)methyl][(3S)-1-methyl-3-pyrrolidinyl]amino}benzonitrile

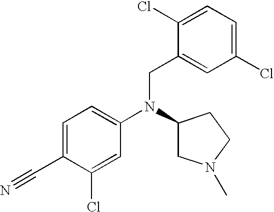

This compound was made according to general procedure of example 78 from 2-(bromomethyl)-1,4-dichlorobenzene. LC-MS (ES) m/e 394 [M+H]⁺.

### Example 205

### 2-chloro-4-{[(2,6-dichlorophenyl)methyl][(3S)-1-methyl-3-pyrrolidinyl]amino}benzonitrile

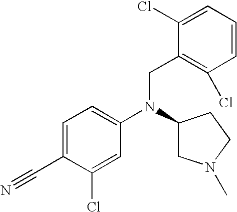

This compound was made according to general procedure of example 78 from 2-(bromomethyl)-1,3-dichlorobenzene. LC-MS (ES) m/e 394.4 [M+H]⁺.

### Example 206

### 2-chloro-4-[[(3S)-1-methyl-3-pyrrolidinyl](2-pyridinylmethyl)amino]benzonitrile

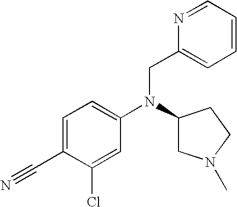

This compound was made according to general procedure of example 78 from 2-(bromomethyl)pyridine. LC-MS (ES) m/e 327.0 [M+H]⁺.

### Example 207

### 2-chloro-4-{[(5-fluoro-2-methylphenyl)methyl][(3S)-1-methyl-3-pyrrolidinyl]amino}benzonitrile

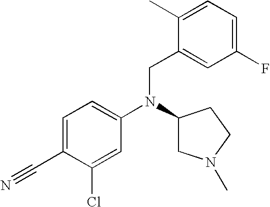

This compound was made according to general procedure of example 78 from 2-(bromomethyl)-4-fluoro-1-methylbenzene. LC-MS (ES) m/e 358.4 [M+H]⁺.

### Example 208

### ethyl N-(3-chloro-4-cyanophenyl)-N-[(3S)-1-methyl-3-pyrrolidinyl]glycinate

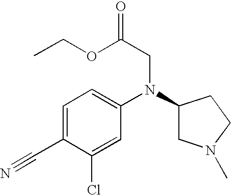

This compound was made according to general procedure of example 78 from ethyl bromoacetate.

LC-MS (ES) m/e 322.4 [M+H]⁺.

### Example 209

### 2-chloro-4-{{[2-chloro-3,4-bis(methyloxy)phenyl]methyl}[(3S)-1-methyl-3-pyrrolidinyl]amino}benzonitrile

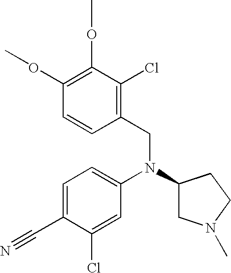

### a) 1-(bromomethyl)-2-chloro-3,4-bis(methyloxy)benzene

PBr₃ (0.13 g, 0.5 mmol) was added dropwise to a solution of [2-chloro-3,4-bis(methyloxy)phenyl]methanol (0.404 g, 2 mmol) in Et₂O (10 mL) and the reaction was stirred at RT for 2 h. NaHCO₃ was added and the reaction was extracted with Et₂O. The organic layer was dried over MgSO₄ and concentrated to yield the crude product (0.5 g, 94%). ¹H-NMR (CDCl₃) δ: 3.903 (d, 6H, J=2.8 Hz), 4.619 (s, 2H), 6.831 (d, 1H, J=8.4 Hz), 7.189 (d, 1H, J=8.8 Hz).

### b) 2-chloro-4-{{[2-chloro-3,4-bis(methyloxy)phenyl]methyl}[(3S)-1-methyl-3-pyrrolidinyl]amino}benzonitrile

This compound was made according to general procedure of example 78 from 1-(bromomethyl)-2-chloro-3,4-bis(methyloxy)benzene. LC-MS (ES) m/e 422.0 [M+H]⁺.

### Example 210

### 4-{{[2-bromo-4,5-bis(methyloxy)phenyl]methyl}[(3)-1-methyl-3-pyrrolidinyl]amino}-2-chlorobenzonitrile

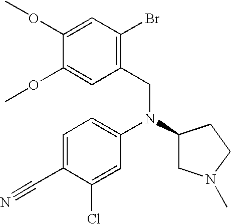

### a) 1-bromo-2-(bromomethyl)-4,5-bis(methyloxy)benzene

A mixture of [3,4-bis(methyloxy)phenyl]methanol (0.84 g, 5 mmol), Br₂ (0.4 mL) and AcOH (4 mL) was stirred at RT for 1 h. The reaction mixture was diluted with CH₂Cl₂ (15 mL) and sat. NaHCO₃ (5 mL). Layers were separated and the organic layer was dried over MgSO₄ and concentrated to yield the product (1.66 g, 100%) as a clear oil. ¹H-NMR (CDCl₃) δ: 3.891 (d, 6H, J=3.2 Hz), 4.601 (s, 2H), 6.941 (s, 1H), 7.029 (s, 1H).

### b) 2-chloro-4-{{[2-chloro-3,4-bis(methyloxy)phenyl]methyl}[(3S)-1-methyl-3-pyrrolidinyl]amino}benzonitrile

This compound was made according to general procedure of example 78 from 1-bromo-2-(bromomethyl)-4,5-bis(methyloxy)benzene. LC-MS (ES) m/e 464.2 [M]⁺.

### Example 211

### 2-chloro-4-{[(6-chloro-1,3-benzodioxol-5-yl)methyl][(3S)-1-methyl-3-pyrrolidinyl]amino}benzonitrile

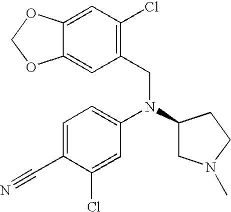

### a) 5-(bromomethyl)-6-chloro-1,3-benzodioxole

This compound was prepared using procedure of example 209 (part a) from (6-chloro-1,3-benzodioxol-5-yl)methanol. ¹H-NMR (CDCl₃) δ: 4.562 (s, 2H), 6.016 (s, 2H), 6.869 (s, 1H), 6.898 (s, 1H).

### b) 2-chloro-4-{{[2-chloro-3,4-bis(methyloxy)phenyl]methyl}[(3S)-1-methyl-3-pyrrolidinyl]amino}benzonitrile

This compound was made according to general procedure of example 78 from 5-(bromomethyl)-6-chloro-1,3-benzodioxole. LC-MS (ES) m/e 405 [M+H]⁺.

### Example 212

### 2-chloro-4-([(3S)-1-(2-thienylmethyl)-3-pyrrolidinyl]{[2-(trifluoromethyl)phenyl]methyl}amino)benzonitrile

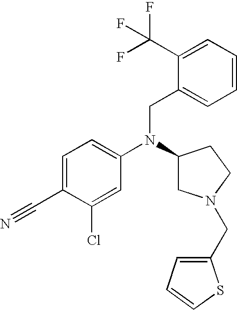

### a) 2-chloro-4-{[(3S)-1-(2-thienylmethyl)-3-pyrrolidinyl]amino}benzonitrile

NaBH(OAc)₃ (1.34 g, 6.3 mmol) was added to an ice cold solution of 2-chloro-4-[(3S)-3-pyrrolidinylamino]benzonitrile (1 g, 4.5 mmol), 2-thiophenecarbaldehyde (0.45 mL, 4.95 mmol) and acetic acid (0.308 mL, 5.4 mmol) in CH₂Cl₂ (30 mL). The reaction was stirred at RT for 3.5 h. Saturated NaHCO₃ was added and the reaction was extracted with EtOAc (4×100 mL). The organic extracts were dried over MgSO₄ and concentrated. The residue was purified by column chromatography (eluted with EtOAc/hexane) to give the titled product (0.9 g, 63%). LC-MS (ES) m/e 318.2 [M+H]⁺.

### b) 2-chloro-4-([(3S)-1-(2-thienylmethyl)-3-pyrrolidinyl]{[2-(trifluoromethyl)phenyl]methyl}amino)benzonitrile

This compound was made according to procedure of example 78 (part c) from 2-chloro-4-{[(3S)-1-(2-thienylmethyl)-3-pyrrolidinyl]amino}benzonitrile and 1-(bromomethyl)-2-(trifluoromethyl)benzene. LC-MS (ES) m/e 476.2 [M+H]⁺.

### Example 213

### 2-chloro-4-{[(2-fluorophenyl)methyl][(3S)-1-(2-thienylmethyl)-3-pyrrolidinyl]amino}benzonitrile

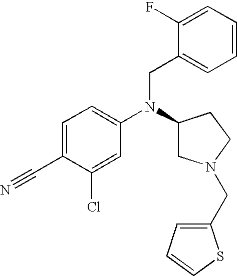

This compound was made according to general procedure of example 212 from 1-(bromomethyl)-2-fluorobenzene. LC-MS (ES) m/e 426.4 [M+H]⁺.

### Example 214

### 4-{[(2-bromophenyl)methyl][(3S)-1-(2-thienyl)methyl)-3-pyrrolidinyl]amino}-2-chlorobenzonitrile

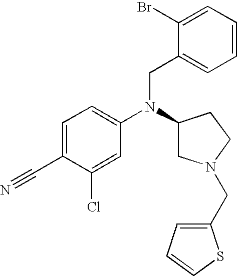

This compound was made according to general procedure of example 212 from 1-bromo-2-(bromomethyl)benzene. LC-MS (ES) m/e 486.4 [M]⁺.

### Example 215

### 2-chloro-4-([(3S)-1-(2-thienylmethyl)-3-pyrrolidinyl]{[3-(trifluoromethyl)phenyl]methyl}amino)benzonitrile

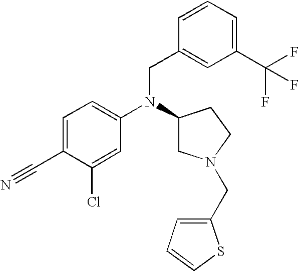

This compound was made according to general procedure of example 212 from 1-(bromomethyl)-3-(trifluoromethyl)benzene. LC-MS (ES) m/e 476.4 [M+H]⁺.

### Example 216

### 2-chloro-4-{ethyl[(3S)-1-(2-thienylmethyl)-3-pyrrolidinyl]amino}benzonitrile

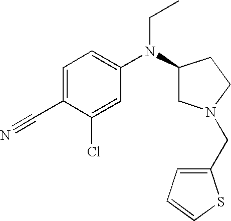

This compound was made according to general procedure of example 212 from iodoethane. LC-MS (ES) m/e 346.4 [M+H]⁺.

### Example 217

### 2-chloro-4-{(2-methyl-2-propen-1-yl)[(3S)-1-(2-thienylmethyl)-3-pyrrolidinyl]amino}benzonitrile

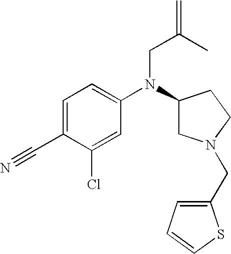

This compound was made according to general procedure of example 212 from 3-bromo-2-methyl-1-propene. LC-MS (ES) m/e 372.4 [M+H]⁺.

### Example 218

### 2-chloro-4-{(2-methylpropyl)[(3S)-1-(2-thienylmethyl)-3-pyrrolidinyl]amino}benzonitrile

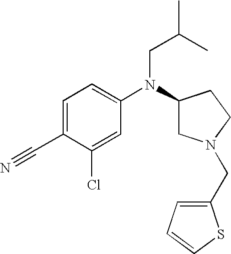

This compound was made according to general procedure of example 212 from 1-bromo-2-methylpropane. LC-MS (ES) m/e 374.2 [M+H]⁺.

### Example 219

### 2-chloro-4-{[(2-chlorophenyl)methyl][(3S)-1-(2-thienylmethyl)-3-pyrrolidinyl]amino}benzonitrile

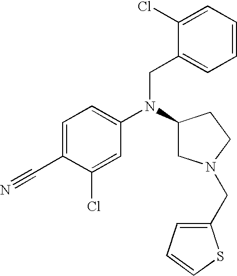

This compound was made according to general procedure of example 212 from 1-(bromomethyl)-2-chlorobenzene. LC-MS (ES) m/e 442.4 [M+H]⁺.

### Example 220

### 2-chloro-4-{(phenylmethyl)[(3S)-1-(2-thienylmethyl)-3-pyrrolidinyl]amino}benzonitrile

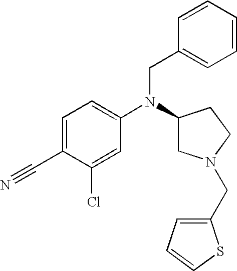

This compound was made according to general procedure of example 212 from (bromomethyl)benzene. LC-MS (ES) m/e 408.4 [M+H]⁺.

### Example 221

### 2-chloro-4-{{[3-(dimethylamino)phenyl]methyl}[(3S)-1-(2-thienylmethyl)-3-pyrrolidinyl]amino}benzonitrile

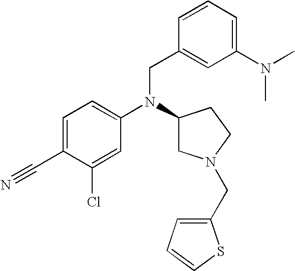

TEA (excess) was added to a solution of [3-(dimethylamino)phenyl]methanol (0.048 g, 0.32 mmol) in benzene (3 mL) with stirring. The reaction was cooled in an ice bath and methanesulfonyl chloride (0.036 g, 0.31 mmol) was added and the reaction solution was stirred at this temperature for 20 min (reaction 1). In reaction flask 2:2-chloro-4-{[(3S)-1-(2-thienylmethyl)-3-pyrrolidinyl]amino}benzonitrile (0.063 g, 0.2 mmol) was dissolved in DMF (2 mL) and cooled to 0° C. NaH (60% in mineral oil, 0.035 g, 0.88 mmol) was added and the reaction mixture was stirred for 30 min. To this mixture, the reaction 1 was added to reaction flask 2 and the reaction mixture was stirred at RT for 18 h. Saturated NH₄Cl was added and the reaction was extracted with EtOAc, dried over Na₂SO₄, and concentrated. The residue was purified via column chromatography (eluted with 0% EtOAc in hexane grading to 35% EtOAc in hexane) to give the titled product (0.069 g, 76%) as white foam. LC-MS (ES) m/e 452.6 [M+H]⁺.

### Example 222

### 2-chloro-4-([(2-chlorophenyl)methyl]{(3S)-1-[(5-methyl-2-furanyl)methyl]-3-pyrrolidinyl}amino)benzonitrile

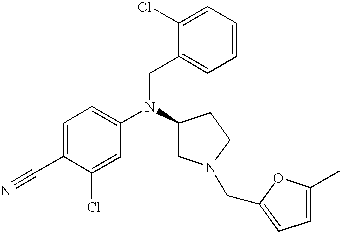

### a) 2-chloro-4-({(3S)-1-[(5-methyl-2-furanyl)methyl]-3-pyrrolidinyl}amino)benzonitrile

This compound was made according to procedure of example 212 (part a) from 5-methyl-2-furancarbaldehyde. LC-MS (ES) m/e 316.2 [M+H]⁺.

### b) 2-chloro-4-([(2-chlorophenyl)methyl]{(3S)-1-[(5-methyl-2-furanyl)methyl]-3-pyrrolidinyl}amino)benzonitrile

This compound was made according to procedure of example 78 (part c) from 2-chloro-4-({(3S)-1-[(5-methyl-2-furanyl)methyl]-3-pyrrolidinyl}amino)benzonitrile and 1-(bromomethyl)-2-chlorobenzene. LC-MS (ES) m/e 440.4 [M+H]⁺.

### Example 223

### 2-chloro-4-[{(3S)-1-[(5-methyl-2-furanyl)methyl]-3-pyrrolidinyl}(phenylmethyl)amino]benzonitrile

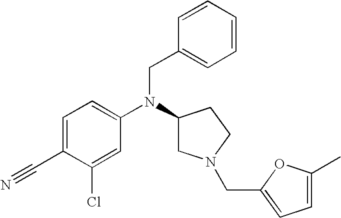

This compound was made according to general procedure of example 222 from (bromomethyl)benzene. LC-MS (ES) m/e 406.6 [M+H]⁺.

### Example 224

### 2-chloro-4-({(3S)-1-[(5-methyl-2-furanyl)methyl]-3-pyrrolidinyl) {}[2-(trifluoromethyl)phenyl]methyl}amino)benzonitrile

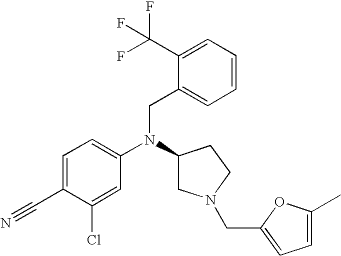

This compound was made according to general procedure of example 222 from 1-(bromomethyl)-2-(trifluoromethyl)benzene. LC-MS (ES) m/e 474.6 [M+H]⁺.

### Example 225

### 2-chloro-4-(ethyl{(3S)-1-[(5-methyl-2-furanyl)methyl]-3-pyrrolidinyl}amino)benzonitrile

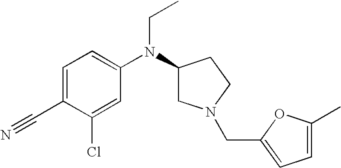

This compound was made according to general procedure of example 222 from iodoethane. LC-MS (ES) m/e 344.2 [M+H]⁺.

### Example 226

### 2-chloro-4-[{(3S)-1-[(5-methyl-2-furanyl)methyl]-3-pyrrolidinyl}(2-methyl-2-propen-1-yl)amino]benzonitrile

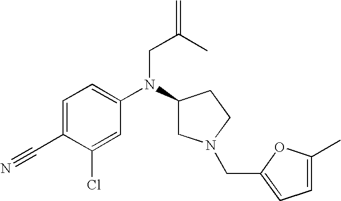

This compound was made according to general procedure of example 222 from 3-bromo-2-methyl-1-propene. LC-MS (ES) m/e 370.2 [M+H]⁺.

### Example 227

### 2-chloro-4-({(3S)-1-[(5-methyl-2-furanyl)methyl]-3-pyrrolidinyl}{[3-(trifluoromethyl)phenyl]methyl}amino)benzonitrile

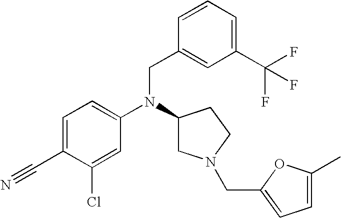

This compound was made according to general procedure of example 222 from 1-(bromomethyl)-3-(trifluoromethyl)benzene. LC-MS (ES) m/e 374.4 [M+H]⁺.

### Example 228

### 4-([(2-bromophenyl)methyl]{(3S)-1-[(5-methyl-2-furanyl)methyl]-3-pyrrolidinyl}amino)-2-chlorobenzonitrile

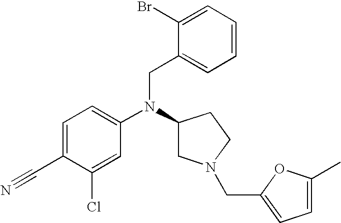

This compound was made according to general procedure of example 222 from 1-bromo-2-(bromomethyl)benzene. LC-MS (ES) m/e 484.2 [M]⁺.

### Example 229

### 2-chloro-4-([(2-fluorophenyl)methyl]{(3S)-1-[(5-methyl-2-furanyl)methyl]-3-pyrrolidinyl}amino)benzonitrile

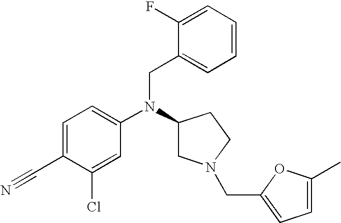

This compound was made according to general procedure of example 222 from 1-(bromomethyl)-2-fluorobenzene. LC-MS (ES) m/e 424.4 [M+H]⁺.

### Example 230

### 2-chloro-4-([(6-chloro-1,3-benzodioxol-5-yl)methyl]{(3S)-1-[(5-methyl-2-furanyl)methyl]-3-pyrrolidinyl}amino)benzonitrile

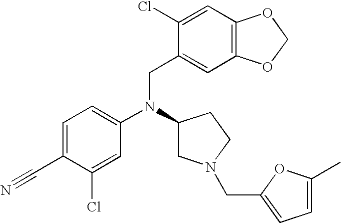

This compound was made according to general procedure of example 222 from 5-(bromomethyl)-6-chloro-1,3-benzodioxole. LC-MS (ES) m/e 484.2 [M+H]⁺.

### Example 231

### 2-chloro-4-([(2-chlorophenyl)methyl]{(3S)-1-[(5-methyl-2-thienyl)methyl]-3-pyrrolidinyl}amino)benzonitrile

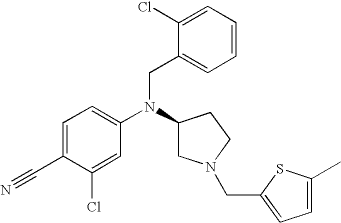

### a) 2-chloro-4-({(3S)-1-[(5-methyl-2-thienyl)methyl]-3-pyrrolidinyl}amino)benzonitrile

This compound was made according to procedure of example 212 (part a) from 5-methyl-2-thiophenecarbaldehyde. LC-MS (ES) m/e 332.2 [M+H]⁺.

### b) 2-chloro-4-([(2-chlorophenyl)methyl]{(3S)-1-[(5-methyl-2-thienyl)methyl]-3-pyrrolidinyl}amino)benzonitrile

This compound was made according to procedure of example 78 (part c) from 2-chloro-4-({(3S)-1-[(5-methyl-2-thienyl)methyl]-3-pyrrolidinyl}amino)benzonitrile and 1-(bromomethyl)-2-chlorobenzene. LC-MS (ES) m/e 456.4 [M+H]⁺.

### Example 232

### 2-chloro-4-({(3S)-1-[(5-methyl-2-thienyl)methyl]-3-pyrrolidinyl}{[2-(trifluoromethyl)phenyl]methyl}amino)benzonitrile

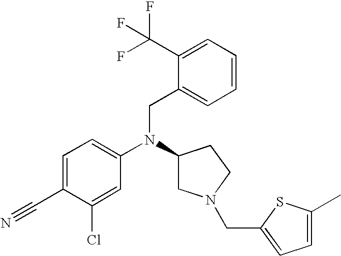

This compound was made according to general procedure of example 231 from 1-(bromomethyl)-2-(trifluoromethyl)benzene. LC-MS (ES) m/e 490.6 [M+H]⁺.

### Example 233

### 2-chloro-4-((2-methylpropyl){(3S)-1-[(5-methyl-2-thienyl)methyl]-3-pyrrolidinyl}amino)benzonitrile

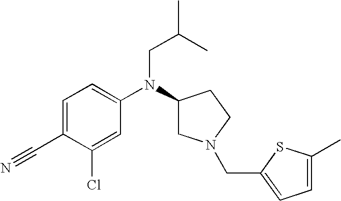

This compound was made according to general procedure of example 231 from 1-bromo-2-methylpropane. LC-MS (ES) m/e 388.4 [M+H]⁺.

### Example 234

### 4-({[2-bromo-4,5-bis(methyloxy)phenyl]methyl}{(3S)-1-[(5-methyl-2-thienyl)methyl]-3-pyrrolidinyl}amino)-2-chlorobenzonitrile

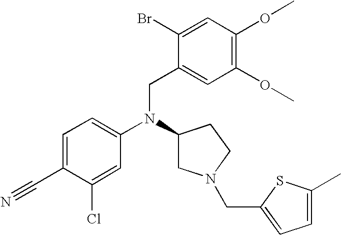

This compound was made according to general procedure of example 231 from 1-bromo-2-(bromomethyl)-4,5-bis(methyloxy)benzene. LC-MS (ES) m/e 560.0 [M]⁺.

### Example 235

### 2-chloro-4-([(2,6-dichlorophenyl)methyl]{(3S)-1-[(5-methyl-2-thienyl)methyl]-3-pyrrolidinyl}amino)benzonitrile

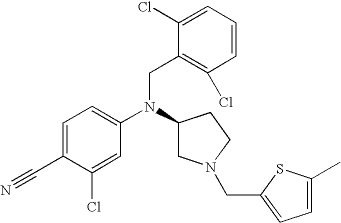

This compound was made according to general procedure of example 231 from 2-(bromomethyl)-1,3-dichlorobenzene. LC-MS (ES) m/e 490.0 [M+H]⁺.

### Example 236

### 2-chloro-4-([(2-chloro-6-fluorophenyl)methyl]{(3S)-1-[(5-methyl-2-thienyl)methyl]-3-pyrrolidinyl}amino)benzonitrile

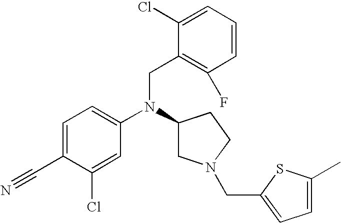

This compound was made according to general procedure of example 231 from 2-(bromomethyl)-1-chloro-3-fluorobenzene. LC-MS (ES) m/e 474.4 [M+H]⁺.

### Example 237

### 2-chloro-4-({(3S)-1-[(3-methyl-2-thienyl)methyl]-3-pyrrolidinyl}{[2-(trifluoromethyl)phenyl]methyl}amino)benzonitrile

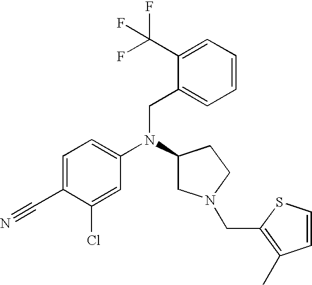

### a) 2-chloro-4-({(3S)-1-[(3-methyl-2-thienyl)methyl]-3-pyrrolidinyl}amino)benzonitrile

This compound was made according to procedure of example 212 (part a) from 3-methyl-2-thiophenecarbaldehyde. LC-MS (ES) m/e 332.2 [M+H]⁺.

### b) 2-chloro-4-({(3S)-1-[(3-methyl-2-thienyl)methyl]-3-pyrrolidinyl}{[2-(trifluoromethyl)phenyl]methyl}amino)benzonitrile

This compound was made according to procedure of example 78 (part c) from 2-chloro-4-({(3S)-1-[(3-methyl-2-thienyl)methyl]-3-pyrrolidinyl}amino)benzonitrile and 1-(bromomethyl)-2-(trifluoromethyl)benzene. LC-MS (ES) m/e 490.6 [M+H]⁺.

### Example 238

### 2-chloro-4-([(2-chlorophenyl)methyl]{(3S)-1-[(3-methyl-2-thienyl)methyl]-3-pyrrolidinyl}amino)benzonitrile

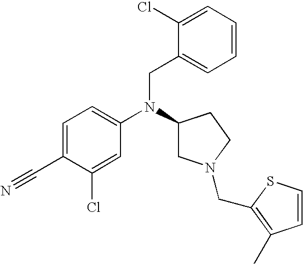

This compound was made according to general procedure of example 237 from 1-(bromomethyl)-2-chlorobenzene. LC-MS (ES) m/e 456.4 [M+H]⁺.

### Example 239

### 2-chloro-4-[{(3S)-1-[(3-methyl-2-thienyl)methyl]-3-pyrrolidinyl}(phenylmethyl)amino]benzonitrile

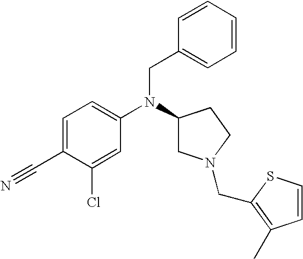

This compound was made according to general procedure of example 237 from (bromomethyl)benzene. LC-MS (ES) m/e 422.4 [M+H]⁺.

### Example 240

### 2-chloro-4-((2-methylpropyl){(3S)-1-[(3-methyl-2-thienyl)methyl]-3-pyrrolidinyl}amino)benzonitrile

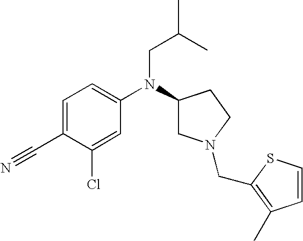

This compound was made according to general procedure of example 237 from 1-bromo-2-methylpropane. LC-MS (ES) m/c 387.8 [M+H]⁺.

### Example 241

### 4-({[2-bromo-4,5-bis(methyloxy)phenyl]methyl}{(3S)-1-[(3-methyl-2-thienyl)methyl]-3-pyrrolidinyl}amino)-2-chlorobenzonitrile

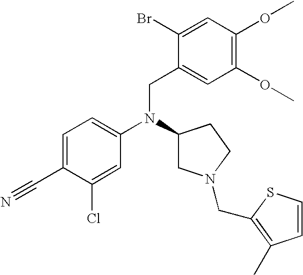

This compound was made according to general procedure of example 237 from 1-bromo-2-(bromomethyl)-4,5-bis(methyloxy)benzene. LC-MS (ES) m/e 560.4 [M]⁺.

### Example 242

### 2-chloro-4-([(6-chloro-1,3-benzodioxol-5-yl)methyl]{(3S)1-[(3-methyl-2-thienyl)methyl]-3-pyrrolidinyl}amino)benzonitrile

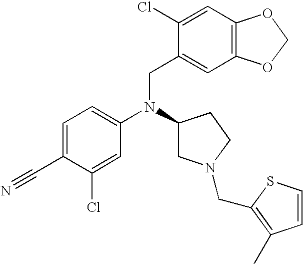

This compound was made according to general procedure of example 237 from 5-(bromomethyl)-6-chloro-1,3-benzodioxole. LC-MS (ES) m/e 501 [M+H]⁺.

### Example 243

### 2-chloro-4-{[(2-chloro-6-fluorophenyl)methyl][(3S)-1-(3,3,3-trifluoropropyl)-3-pyrrolidinyl]amino}benzonitrile

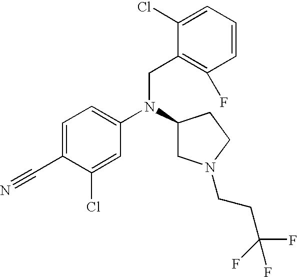

### a) 2-chloro-4-{[(3S)-1-(3,3,3-trifluoropropyl)-3-pyrrolidinyl]amino}benzonitrile

K₂CO₃ (1.313 g, 9.50 mmol) was added to a solution of 2-chloro-4-[(3S)-3-pyrrolidinylamino]benzonitrile (0.7 g, 3.17 mmol) in MeCN (20 mL) and the reaction was heated to 55° C. 1,1,1-Trifluoro-3-iodopropane (2.84 g, 12.67 mmol) was added dropwise and the reaction mixture was stirred for 24 h. After cooling, saturated NaHCO₃ was added and the reaction was extracted with EtOAc. The organic layer was dried over MgSO₄ and concentrated to yield the product (0.72 g, 72%). LC-MS (ES) m/e 318.2 [M+H]⁺.

### b) 2-chloro-4-{[(2-chloro-6-fluorophenyl)methyl][(3S)-1-(3,3,3-trifluoropropyl)-3-pyrrolidinyl]amino}benzonitrile

This compound was made according to procedure of example 78 (part c) from 2-chloro-4-{[(3S)-1-(3,3,3-trifluoropropyl)-3-pyrrolidinyl]amino}benzonitrile and 2-(bromomethyl)-1-chloro-3-fluorobenzene. LC-MS (ES) m/e 460.6 [M+H]⁺.

### Example 244

### 2-chloro-4-{(phenylmethyl)[(3S)-1-(3,3,3-trifluoropropyl)-3-pyrrolidinyl]amino}benzonitrile

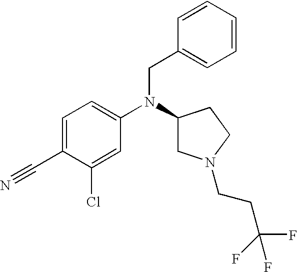

This compound was made according to general procedure of example 243 from (bromomethyl)benzene. LC-MS (ES) m/e 408.4 [M+H]⁺.

### Example 245

### 2-chloro-4-{[(2-methylphenyl)methyl][(3S)-1-(3,3,3-trifluoropropyl)-3-pyrrolidinyl]amino}benzonitrile

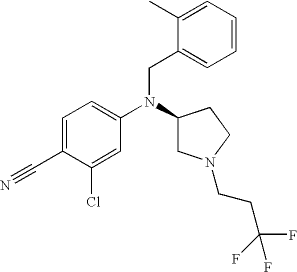

This compound was made according to general procedure of example 243 from 1-(bromomethyl)-2-methylbenzene. LC-MS (ES) m/e 422.0 [M+H]⁺.

### Example 246

### 2-chloro-4-{[(5-fluoro-2-methylphenyl)methyl][(3S)-1-(3,3,3-trifluoropropyl)-3-pyrrolidinyl]amino}benzonitrile

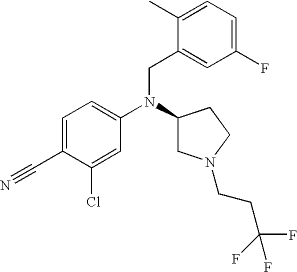

This compound was made according to general procedure of example 243 from 2-(bromomethyl)-4-fluoro-1-methylbenzene. LC-MS (ES) m/e 440.4 [M+H]⁺.

### Example 247

### 2-chloro-4-{{[2-(trifluoromethyl)phenyl]methyl}[(3S)-1-(3,3,3-trifluoropropyl)-3-pyrrolidinyl]amino}benzonitrile

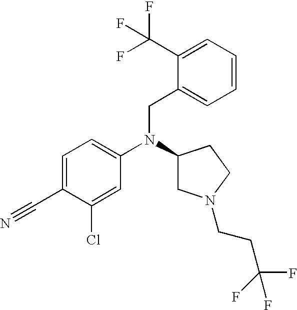

This compound was made according to general procedure of example 243 from 1-(bromomethyl)-2-(trifluoromethyl)benzene. LC-MS (ES) m/e 476.4 [M+H]⁺.

### Example 248

### 4-{[(2-bromophenyl)methyl][(3S)-1-(3,3,3-trifluoropropyl)-3-pyrrolidinyl]amino}-2-chlorobenzonitrile

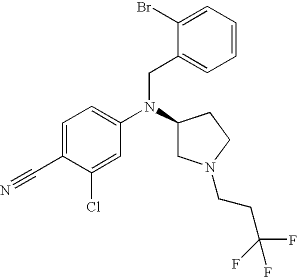

This compound was made according to general procedure of example 243 from 1-bromo-2-(bromomethyl)benzene. LC-MS (ES) m/e 486.0 [M]⁺.

### Example 249

### 2-chloro-4-{ethyl[(3S)-1-(3,3,3-trifluoropropyl)-3-pyrrolidinyl]amino}benzonitrile

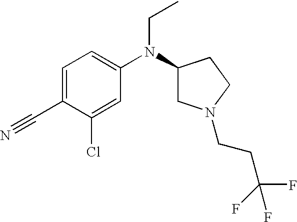

This compound was made according to general procedure of example 243 from iodoethane. LC-MS (ES) m/e 346.0 [M+H]⁺.

### Example 250

### 2-chloro-4-{(2-methylpropyl)[(3S)-1-(3,3,3-trifluoropropyl)-3-pyrrolidinyl]amino}benzonitrile

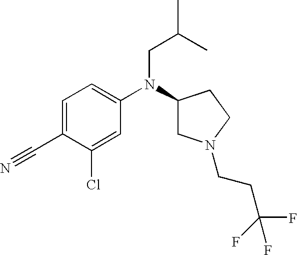

This compound was made according to general procedure of example 243 from 1-bromo-2-methylpropane. LC-MS (ES) m/c 374.2 [M+H]⁺.

### Example 251

### 2-chloro-4-{[(2-fluorophenyl)methyl][(3S)-1-(3,3,3-trifluoropropyl)-3-pyrrolidinyl]amino}benzonitrile

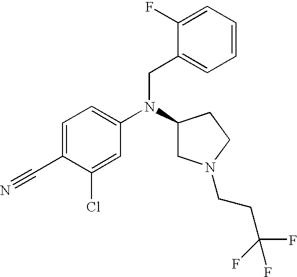

This compound was made according to general procedure of example 243 from 1-(bromomethyl)-2-fluorobenzene. LC-MS (ES) m/e 426.2 [M+H]⁺.

### Example 252

### 4-{{[2-bromo-4,5-bis(methyloxy)phenyl]methyl}[(3S)-1-(3,3,3-trifluoropropyl)-3-pyrrolidinyl]amino}-2-chlorobenzonitrile

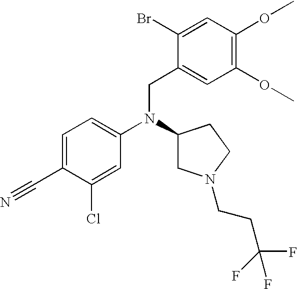

This compound was made according to general procedure of example 243 from 1-bromo-2-(bromomethyl)-4,5-bis(methyloxy)benzene. LC-MS (ES) m/e 548.0 [M+H]⁺.

### Example 253

### 2-chloro-4-{[(6-chloro-1,3-benzodioxol-5-yl)methyl][(3S)-1-(3,3,3-trifluoropropyl)-3-pyrrolidinyl]amino}benzonitrile

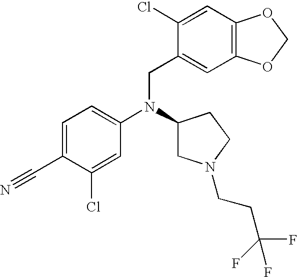

This compound was made according to general procedure of example 243 from 5-(bromomethyl)-6-chloro-1,3-benzodioxole. LC-MS (ES) m/e 486.4 [M+H]⁺.

### Example 254

### 4-{[(2-methylphenyl)methyl][(3S)-1-methyl-3-pyrrolidinyl]amino}benzonitrile

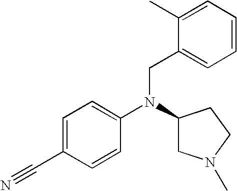

This compound was prepared according to procedure of example 8 (part a) from 2-chloro-4-{[(2-methylphenyl)methyl][(3S)-1-methyl-3-pyrrolidinyl]amino}benzonitrile. LC-MS (ES) m/c 306.4 [M+H]⁺.

### Example 255

### 4-{[(2-fluorophenyl)methyl][(3S)-1-methyl-3-pyrrolidinyl]amino}benzonitrile

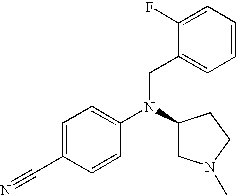

This compound was prepared according to procedure of example 8 (part a) from 2-chloro-4-{[(2-fluorophenyl)methyl][(3S)-1-methyl-3-pyrrolidinyl]amino}benzonitrile. LC-MS (ES) m/e 310.0 [M+H]⁺.

### Example 256

### 4-[((3S)-3-{(3-chloro-4-cyanophenyl)[(2-methylphenyl)methyl]amino}-1-pyrrolidinyl)methyl]benzoic acid

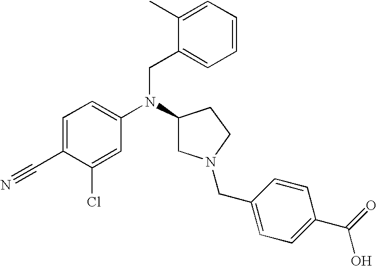

This compound was made according to general procedure of example 17 from 2-chloro-4-{[(2-methylphenyl)methyl][(3S)-3-pyrrolidinyl]amino}benzonitrile and 4-formylbenzoic acid. LC-MS (ES) m/e 460.2 [M+H]⁺.

### Example 257

### 3-[((3S)-3-{(3-chloro-4-cyanophenyl)[(2-methylphenyl)methyl]amino}-1-pyrrolidinyl)methyl]benzoic acid

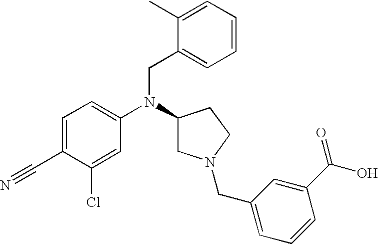

This compound was made according to general procedure of example 17 from 2-chloro-4-{[(2-methylphenyl)methyl][(3S)-3-pyrrolidinyl]amino}benzonitrile and 3-formylbenzoic acid. LC-MS (ES) m/e 460.6 [M+H]⁺.

### Example 258

### 2-chloro-4-{[(2-methylphenyl)methyl][(3S)-1-(tetrahydro-3-furanylmethyl)-3-pyrrolidinyl]amino}benzonitrile

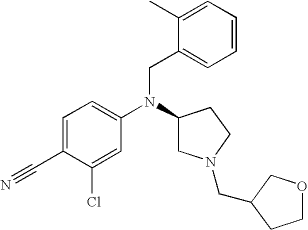

This compound was made according to general procedure of example 17 from 2-chloro-4-{[(2-methylphenyl)methyl][(3S)-3-pyrrolidinyl]amino}benzonitrile and tetrahydro-3-furancarbaldehyde.

LC-MS (ES) m/e 410.6 [M+H]⁺.

### Example 259

### 2-chloro-4-{{(3S)-1-[(2,4-dioxo-1,2,3,4-tetrahydro-5-pyrimidinyl)methyl]-3-pyrrolidinyl}[(2-methylphenyl)methyl]amino}benzonitrile

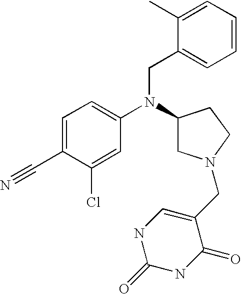

This compound was made according to general procedure of example 17 from 2-chloro-4-{[(2-methylphenyl)methyl][(3S)-3-pyrrolidinyl]amino}benzonitrile and 2,4-dioxo-1,2,3,4-tetrahydro-5-pyrimidinecarbaldehyde. LC-MS (ES) m/e 450.6 [M+H]⁺.

### Example 260

### 2-chloro-4-{[(3S)-1-(2-hydroxyethyl)-3-pyrrolidinyl][(2-methylphenyl)methyl]-amino}benzonitrile

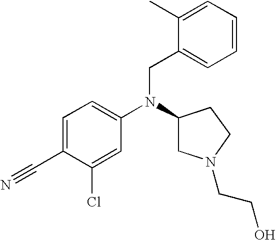

This compound was made according to general procedure of example 17 from 2-chloro-4-{[(2-methylphenyl)methyl][(3S)-3-pyrrolidinyl]amino}benzonitrile and hydroxyacetaldehyde. LC-MS (ES) m/e 370.4 [M+H]⁺.

### Example 261

### 2-chloro-4-{[(3S)-1-(3-hydroxybutyl)-3-pyrrolidinyl][(2-methylphenyl)methyl]amino}benzonitrile

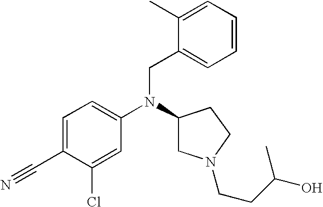

This compound was made according to general procedure of example 17 from 2-chloro-4-{[(2-methylphenyl)methyl][(3S)-3-pyrrolidinyl]amino}benzonitrile and 3-hydroxybutanal. LC-MS (ES) m/e 398.4 [M+H]⁺.

### Example 262

### 2-chloro-4-{[(3S)-1-ethyl-3-pyrrolidinyl][(2-methylphenyl)methyl]amino}benzonitrile

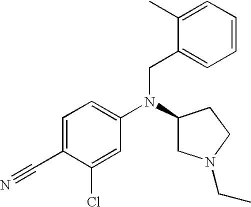

This compound was made according to general procedure of example 17 from 2-chloro-4-{[(2-methylphenyl)methyl][(3S)-3-pyrrolidinyl]amino}benzonitrile and acetaldehyde. LC-MS (ES) m/e 354.2 [M+H]⁺.

### Example 263

### 2-chloro-4-{{(3S)-1-[(2,4-dimethyl-1,3-thiazol-5-yl)methyl]-3-pyrrolidinyl}[(2-methylphenyl)methyl]amino}benzonitrile

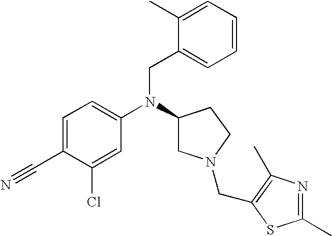

This compound was made according to general procedure of example 17 from 2-chloro-4-{[(2-methylphenyl)methyl][(3S)-3-pyrrolidinyl]amino}benzonitrile and 2,4-dimethyl-1,3-thiazole-5-carbaldehyde. LC-MS (ES) m/e 452.0 [M+H]⁺.

### Example 264

### 2-chloro-4-({(3S)-1-[(2,4-dioxo-1,2,3,4-tetrahydro-5-pyrimidinyl)methyl]-3-pyrrolidinyl}{[2-(trifluoromethyl)phenyl]methyl}amino)benzonitrile

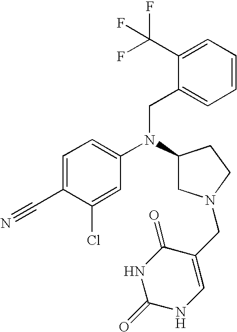

This compound was made according to general procedure of example 17 from 2,4-dioxo-1,2,3,4-tetrahydro-5-pyrimidinecarbaldehyde. LC-MS (ES) m/e 504.4 [M+H]⁺.

### Example 265

### 4-[((3S)-3-{(3-chloro-4-cyanophenyl)[(2-methylphenyl)methyl]amino}-1-pyrrolidinyl)methyl]-3-hydroxybenzoic acid

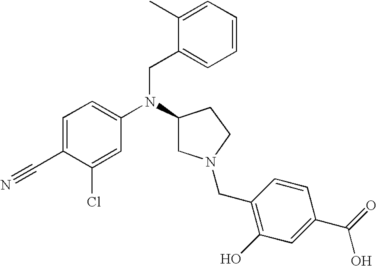

This compound was made according to general procedure of example 17 from 2-chloro-4-{[(2-methylphenyl)methyl][(3S)-3-pyrrolidinyl]amino}benzonitrile and 4-formyl-3-hydroxybenzoic acid. LC-MS (ES) m/e 476.2 [M+H]⁺.

### Example 266

### 2-chloro-4-{{(3S)-1-[(3,5-dimethyl-1H-pyrrol-2-yl)methyl]-3-pyrrolidinyl}[(2-methylphenyl)methyl]amino}benzonitrile

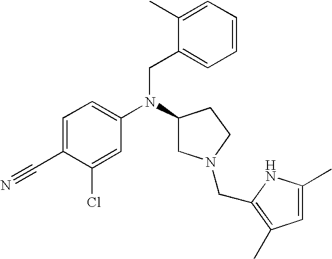

This compound was made according to general procedure of example 17 from 2-chloro-4-{[(2-methylphenyl)methyl][(3S)-3-pyrrolidinyl]amino}benzonitrile and 3,5-dimethyl-1H-pyrrole-2-carbaldehyde. LC-MS (ES) m/e 435.4 [M+H]⁺.

### Example 267

### 2-chloro-4-{{(3S)-1-[(1,3-dimethyl-1H-pyrazol-5-yl)methyl]-3-pyrrolidinyl}[(2-methylphenyl)methyl]amino}benzonitrile

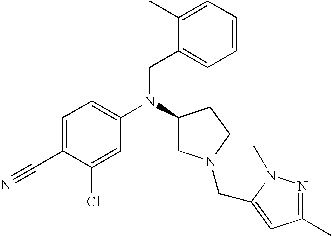

This compound was made according to general procedure of example 17 from 2-chloro-4-{[(2-methylphenyl)methyl][(3S)-3-pyrrolidinyl]amino}benzonitrile and 1,3-dimethyl-1H-pyrazole-5-carbaldehyde. LC-MS (ES) m/e 434.0 [M+H]⁺.

### Example 268

### 2-chloro-4-([(2-chlorophenyl)methyl]{(3S)-1-[(2,4-dioxo-1,2,3,4-tetrahydro-5-pyrimidinyl)methyl]-3-pyrrolidinyl}amino)benzonitrile

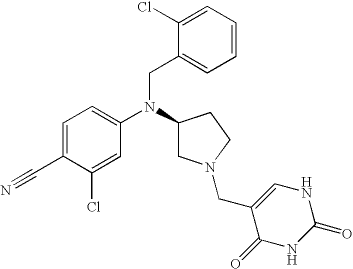

This compound was made according to general procedure of example 17 from 2-chloro-4-{[(2-chlorophenyl)methyl][(3S)-3-pyrrolidinyl]amino}benzonitrile and 2,4-dioxo-1,2,3,4-tetrahydro-5-pyrimidinecarbaldehyde. LC-MS (ES) m/e 470.2 [M+H]⁺.

### Example 269

### 2-chloro-4-{[(3S)-1-(1H-imidazol-4-ylmethyl)-3-pyrrolidinyl][(2-methylphenyl)methyl]amino}benzonitrile

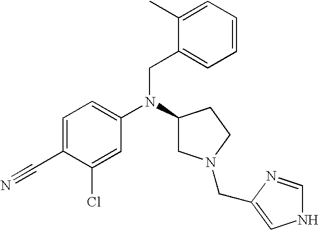

This compound was made according to general procedure of example 17 from 2-chloro-4-{[(2-methylphenyl)methyl][(3S)-3-pyrrolidinyl]amino}benzonitrile and 1H-imidazole-4-carbaldehyde.

LC-MS (ES) m/e 406.4 [M+H]⁺.

### Example 270

### 2-chloro-4-{{(3S)-1-[(2,4-dioxo-1,2,3,4-tetrahydro-5-pyrimidinyl)methyl]-3-pyrrolidinyl}[(2-fluorophenyl)methyl]amino}benzonitrile

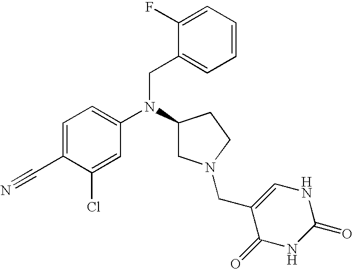

This compound was made according to general procedure of example 17 from 2-chloro-4-{[(2-fluorophenyl)methyl][(3S)-3-pyrrolidinyl]amino}benzonitrile and 2,4-dioxo-1,2,3,4-tetrahydro-5-pyrimidinecarbaldehyde. LC-MS (ES) m/e 453.8 [M+H]⁺.

### Example 271

### 2-[((3S)-3-{(3-chloro-4-cyanophenyl)[(2-methylphenyl)methyl]amino}-1-pyrrolidinyl)methyl]benzoic acid

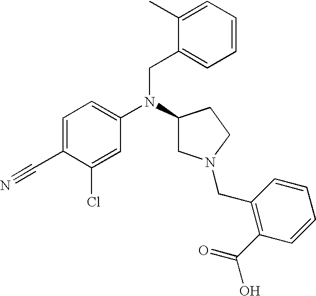

This compound was made according to general procedure of example 17 from 2-chloro-4-{[(2-methylphenyl)methyl][(3S)-3-pyrrolidinyl]amino}benzonitrile and 2-formylbenzoic acid. LC-MS (ES) m/e 460.4 [M+H]⁺.

### Example 272

### 2-chloro-4-{[(2-methylphenyl)methyl][(3S)-1-(1H-pyrazol-3-ylmethyl)-3-pyrrolidinyl]amino}benzonitrile

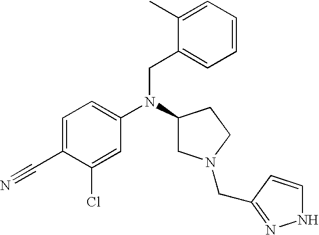

This compound was made according to general procedure of example 17 from 2-chloro-4-{[(2-methylphenyl)methyl][(3S)-3-pyrrolidinyl]amino}benzonitrile and 1H-pyrazole-4-carbaldehyde.

LC-MS (ES) m/e 406.4 [M+H]⁺.

### Example 273

### 5-[((3S)-3-{(3-chloro-4-cyanophenyl)[(2-methylphenyl)methyl]amino}-1-pyrrolidinyl)methyl]-2-hydroxybenzoic acid

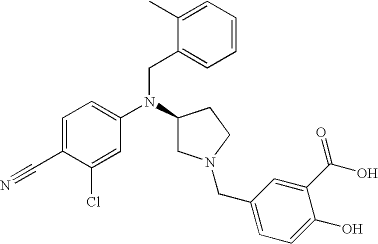

This compound was made according to general procedure of example 17 from 2-chloro-4-{[(2-methylphenyl)methyl][(3S)-3-pyrrolidinyl]amino}benzonitrile and 5-formyl-2-hydroxybenzoic acid. LC-MS (ES) m/c 476.2 [M+H]⁺.

### Example 274

### methyl 4-[((3S)-3-{(3-chloro-4-cyanophenyl)[(2-methylphenyl)methyl]amino}-1-pyrrolidinyl)methyl]-3-nitrobenzoate

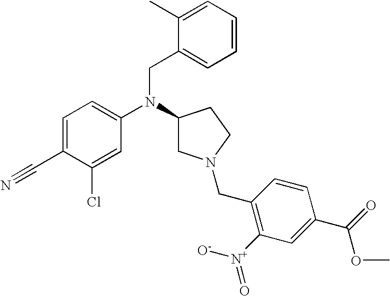

This compound was made according to general procedure of example 17 from 2-chloro-4-{[(2-methylphenyl)methyl][(3S)-3-pyrrolidinyl]amino}benzonitrile and methyl 3-formyl-4-nitrobenzoate. LC-MS (ES) m/e 519.2 [M+H]⁺.

### Example 275

### 2-chloro-4-({(3S)-1-[(1-methyl-1H-pyrazol-5-yl)methyl]-3-pyrrolidinyl}{[2-(trifluoromethyl)phenyl]methyl}amino)benzonitrile

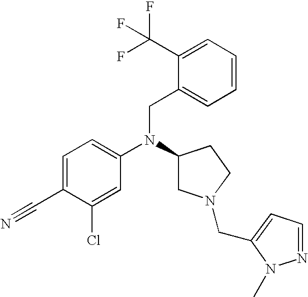

This compound was made according to general procedure of example 17 from 1-methyl-1H-pyrazole-5-carbaldehyde. LC-MS (ES) m/e 474.6 [M+H]⁺.

### Example 276

### 2-chloro-4-([(3S)-1-(2-pyrazinylmethyl)-3-pyrrolidinyl]{[2-(trifluoromethyl)phenyl]methyl}amino)benzonitrile

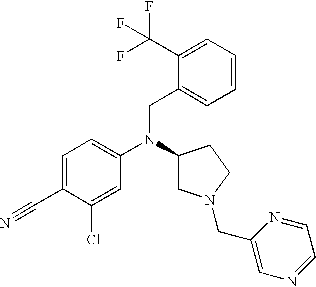

This compound was made according to general procedure of example 17 from 2-pyrazinecarbaldehyde. LC-MS (ES) m/e 472.4 [M+H]⁺.

### Example 277

### 2-chloro-4-([(3S)-1-(5-pyrimidinylmethyl)-3-pyrrolidinyl]{[2-(trifluoromethyl)phenyl]methyl}amino)benzonitrile

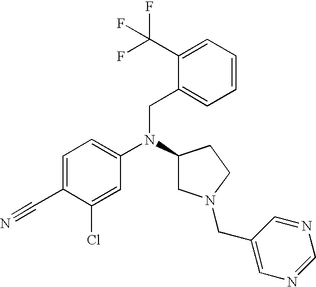

This compound was made according to general procedure of example 17 from 5-pyrimidinecarbaldehyde. LC-MS (ES) m/e 472.6 [M+H]⁺.

### Example 278

### 2-chloro-4-({(3S)-1-[(1-methyl-1H-pyrazol-4-yl)methyl]-3-pyrrolidinyl}{[2-(trifluoromethyl)phenyl]methyl}amino)benzonitrile

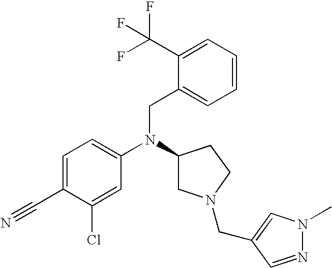

This compound was made according to general procedure of example 17 from 1-methyl-1H-pyrazole-4-carbaldehyde. LC-MS (ES) m/e 474.2 [M+H]⁺.

### Example 279

### 2-chloro-4-([(3S)-1-(2,2-dimethylpropyl)-3-pyrrolidinyl]{[2-(trifluoromethyl)phenyl]methyl}amino)benzonitrile

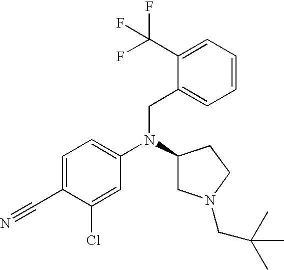

This compound was made according to general procedure of example 17 from 2,2-dimethylpropanal. LC-MS (ES) m/e 450.4 [M+H]⁺.

### Example 280

### methyl 3-{[(3S)-1-((3-chloro-4-cyanophenyl){[2-(trifluoromethyl)phenyl]methyl}amino)-1-pyrrolidinyl]methyl}benzoate

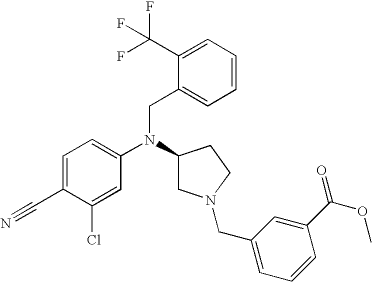

This compound was made according to general procedure of example 17 from methyl 3-formylbenzoate. LC-MS (ES) m/e 528.4 [M+H]⁺.

### Example 281

### 2-chloro-4-{[(2-chlorophenyl)methyl][(3S)-1-(2,2-dimethylpropyl)-3-pyrrolidinyl]amino}benzonitrile

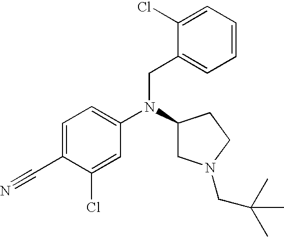

This compound was made according to general procedure of example 17 from 2-chloro-4-{[(2-chlorophenyl)methyl][(3S)-3-pyrrolidinyl]amino}benzonitrile and methyl 3-formylbenzoate. LC-MS (ES) m/e 416.2 [M+H]⁺.

### Example 282

### 2-chloro-4-([(3S)-1-(2,2,2-trifluoroethyl)-3-pyrrolidinyl]{[2-(trifluoromethyl)phenyl]methyl}amino)benzonitrile

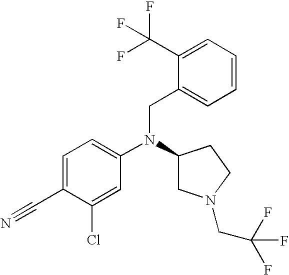

This compound was made according to general procedure of example 41 from 2-bromo-1,1,1-trifluoroethane. LC-MS (ES) m/e 462.4 [M+H]⁺.

### Example 283

### 1,1-dimethylethyl((3S)-3-{(3-chloro-4-cyanophenyl)[(2-chlorophenyl)methyl]amino}-1-pyrrolidinyl)acetate

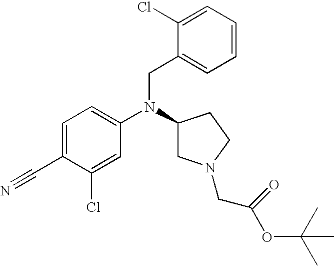

This compound was made according to general procedure of example 41 from 2-chloro-4-{[(2-chlorophenyl)methyl][(3S)-3-pyrrolidinyl]amino}benzonitrile and 1,1-dimethylethyl bromoacetate.

LC-MS (ES) m/e 460.4 [M+H]⁺.

### Example 284

### 2-chloro-4-{[(3S)-1-(3-hydroxypropyl)-3-pyrrolidinyl][(2-methylphenyl)methyl]amino}benzonitrile

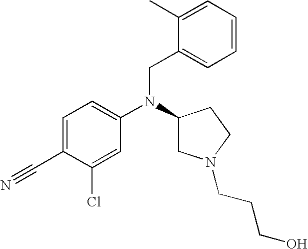

This compound was made according to general procedure of example 41 from 2-chloro-4-{[(2-methylphenyl)methyl][(3S)-3-pyrrolidinyl]amino}benzonitrile and 3-bromo-1-propanol. LC-MS (ES) m/e 384.2 [M+H]⁺.

### Example 285

### 2-chloro-4-{[(3S)-1-(2-cyanoethyl)-3-pyrrolidinyl][(2-methylphenyl)methyl]amino}benzonitrile

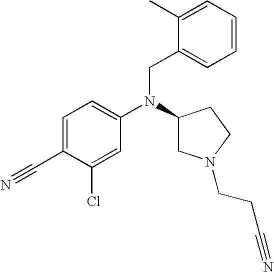

This compound was made according to general procedure of example 41 from 2-chloro-4-{[(2-methylphenyl)methyl][(3S)-3-pyrrolidinyl]amino}benzonitrile and 3-bromopropanenitrile. LC-MS (ES) m/e 379.2 [M+H]⁺.

### Example 286

### 2-chloro-4-{{(3S)-1-[2-(1,3-dioxolan-2-yl)ethyl]-3-pyrrolidinyl}[(2-methylphenyl)methyl]amino}benzonitrile

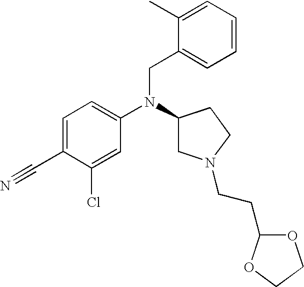

This compound was made according to general procedure of example 41 from 2-chloro-4-{[(2-methylphenyl)methyl][(3S)-3-pyrrolidinyl]amino}benzonitrile and 2-(2-bromoethyl)-1,3-dioxolane.

LC-MS (ES) m/e 426.4 [M+H]⁺.

### Example 287

### 3-((3S)-3-{(3-chloro-4-cyanophenyl)[(2-methylphenyl)methyl]amino}-1-pyrrolidinyl)propanamide

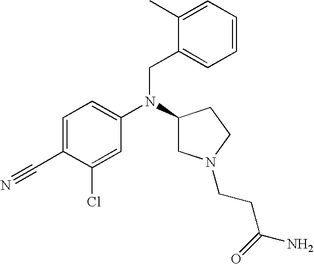

This compound was made according to general procedure of example 41 from 2-chloro-4-{[(2-methylphenyl)methyl][(3S)-3-pyrrolidinyl]amino}benzonitrile and 3-bromopropanamide. LC-MS (ES) m/e 397 [M+H]⁺.

### Example 288

### 3-{[(3S)-3-((3-chloro-4-cyanophenyl){[2-(trifluoromethyl)phenyl]methyl}amino)-1-pyrrolidinyl]methyl}benzoic acid

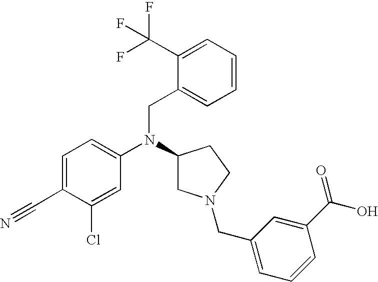

A mixture of methyl 3-{[(3S)-3-((3-chloro-4-cyanophenyl){[2-(trifluoromethyl)phenyl]methyl}amino)-1-pyrrolidinyl]methyl}benzoate (0.04 g, 0.08 mmol), EtOH (2 mL), H₂O (0.5 mL) and 1N NaOH (0.25 mL) was heated with stirring at 55° C. for 2 h. The reaction was cooled and diluted with H₂O (5 mL) and acidified to pH˜5 with 1N HCl. The white solid precipitate was filtered, washed with H₂O, and dried to give the product (0.033 g, 85%).

LC-MS (ES) m/e 514.6 [M+H]⁺.

### Example 289

### N-(3,4-dichlorophenyl)-1-methyl-N-[(2-methylphenyl)methyl]-3-pyrrolidinamine

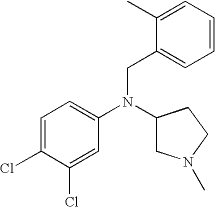

### a) 1,1-dimethylethyl 3-[(3,4-dichlorophenyl)amino]-1-pyrrolidinecarboxylate

A mixture of 3,4-dichloroaniline (0.175 g, 1.1 mmol), 1,1-dimethylethyl 3-oxo-1-pyrrolidinecarboxylate (0.1 g, 0.54 mmol) and NaBH₃CN (0.134 g, 2.16 mmol) in DMF was stirred in a microwave synthesizer at 70° C. for 30 min. The reaction was partitioned between H₂O and EtOAc and the organic layer was dried over MgSO₄ and concentrated. The residue was purified by column chromatography to give the product. LC-MS (ES) m/e 275.2 [M−56]⁺.

### b) N-(3,4-dichlorophenyl)-N-[(2-methylphenyl)methyl]-3-pyrrolidinamine

This compound was prepared according to procedure of example 1 part b and c from 1,1-dimethylethyl 3-[(3,4-dichlorophenyl)amino]-1-pyrrolidinecarboxylate and 1-(bromomethyl)-2-methylbenzene. LC-MS (ES) m/e 335.2 [M+H]⁺.

### c) N-(3,4-dichlorophenyl)-1-methyl-N-[(2-methylphenyl)methyl]-3-pyrrolidinamine

This compound was made according to general procedure of example 9 from N-(3,4-dichlorophenyl)-N-[(2-methylphenyl)methyl]-3-pyrrolidinamine. LC-MS (ES) m/e 349 [M+H]⁺.

### Example 290

### N-(3,4-dichlorophenyl)-N-[(2-methylphenyl)methyl]-1-(methylsulfonyl)-3-pyrrolidinamine

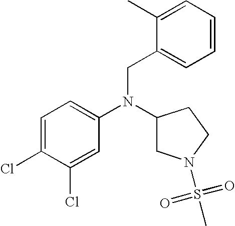

This compound was made according to procedure of example 77 from N-(3,4-dichlorophenyl)-N-[(2-methylphenyl)methyl]-3-pyrrolidinamine. LC-MS (ES) m/e 413.0 [M+H]⁺.

### Example 291

### N-[3-chloro-4-(methyloxy)phenyl]-N-[(2-methylphenyl)methyl]-1-(methylsulfonyl)-3-pyrrolidinamine

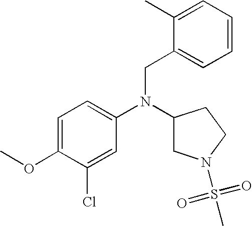

### a) 1,1-dimethylethyl 3-{[3-chloro-4-(methyloxy)phenyl]amino}-1-pyrrolidinecarboxylate

This compound was made according to procedure of example 289 part a from 3-chloro-4-(methyloxy)aniline. LC-MS (ES) m/c 326.4 [M+H]⁺.

### b) N-[3-chloro-4-(methyloxy)phenyl]-N-[(2-methylphenyl)methyl]-3-pyrrolidinamine

This compound was made according to procedure of example 1 (part b and c) from 1,1-dimethylethyl 3-{[3-chloro-4-(methyloxy)phenyl]amino}-1-pyrrolidinecarboxylate and 1-(bromomethyl)-2-methylbenzene. LC-MS (ES) m/c 331.4 [M+H]⁺.

### c) N-[3-chloro-4-(methyloxy)phenyl]-N-[(2-methylphenyl)methyl]-1-(methylsulfonyl)-3-pyrrolidinamine

This compound was made according to procedure of example 77 from N-[3-chloro-4-(methyloxy)phenyl]-N-[(2-methylphenyl)methyl]-3-pyrrolidinamine. LC-MS (ES) m/e 409.6 [M+H]⁺.

### Example 292

### (3S)—N-[(2-methylphenyl)methyl]-1-(methylsulfonyl)-N-[4-nitro-3-(trifluoromethyl)phenyl]-3-pyrrolidinamine

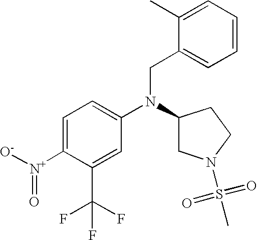

### a) 1,1-dimethylethyl (3S)-3-{[4-nitro-3-(trifluoromethyl)phenyl]amino}-1-pyrrolidinecarboxylate

1,1-Dimethylethyl (3S)-3-amino-1-pyrrolidinecarboxylate (0.534 g, 2.87 mmol) was added to an ice cold suspension of NaH (60% in mineral oil, 0.384 g, 9.6 mmol) in DMF. After stirring for 10 min, 4-fluoro-1-nitro-2-(trifluoromethyl)benzene (0.5 g, 2.4 mmol) was added and the reaction mixture was stirred at 0° C. for 2 h. The reaction was quenched with H₂O and then partitioned between H₂O and EtOAc. The organic layer was dried over MgSO₄, concentrated, and purified via HPLC to give the product. LC-MS (ES) m/e 376.6 [M+H]⁺.

### b) (3S)—N-[(2-methylphenyl)methyl]-N-[4-nitro-3-(trifluoromethyl)phenyl]-3-pyrrolidinamine

This compound was made according to procedure of example 1 (part b and c) from 1,1-dimethylethyl (3S)-3-{[4-nitro-3-(trifluoromethyl)phenyl]amino}-1-pyrrolidinecarboxylate and 1-(bromomethyl)-2-methylbenzene. LC-MS (ES) m/e 380.6 [M+H]⁺.

### c) (3S)—N-[(2-methylphenyl)methyl]-1-(methylsulfonyl)-N-[4-nitro-3-(trifluoromethyl)phenyl]-3-pyrrolidinamine

This compound was made according to procedure of example 77 from N-[3-chloro-4-(methyloxy)phenyl]-N-[(2-methylphenyl)methyl]-3-pyrrolidinamine. LC-MS (ES) m/e 458.2 [M+H]⁺.

### Example 293

### 2-chloro-4-{[(5-chloro-2-fluorophenyl)methyl][(3S)-1-(methylsulfonyl)-3-pyrrolidinyl]amino}benzonitrile

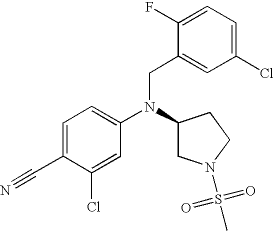

This compound was made according to general procedure of example 105 from 2-(bromomethyl)-4-chloro-1-fluorobenzene. LC-MS (ES) m/e 442.4 [M+H]⁺.

### Example 294

### 2-chloro-4-{[(2,4-dichloro-5-fluorophenyl)methyl][(3S)-1-(methylsulfonyl)-3-pyrrolidinyl]amino}benzonitrile

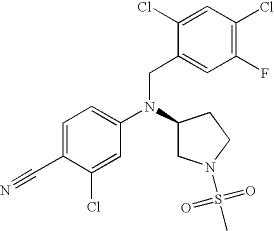

This compound was made according to general procedure of example 105 from 1-(bromomethyl)-2,4-dichloro-5-fluorobenzene. LC-MS (ES) m/e 476.0 [M+H]⁺.

### Example 295

### 2-chloro-4-{[(2-chloro-3,6-difluorophenyl)methyl][(3S)-1-(methylsulfonyl)-3-pyrrolidinyl]amino}benzonitrile

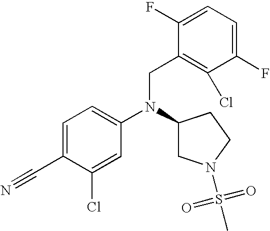

This compound was made according to general procedure of example 105 from 2-(bromomethyl)-3-chloro-1,4-difluorobenzene. LC-MS (ES) m/e 460.1 [M+H]⁺.

### Example 296

### 2-chloro-4-{[(2,3-difluoro-4-methylphenyl)methyl][(3S)-1-(methylsulfonyl)-3-pyrrolidinyl]amino}benzonitrile

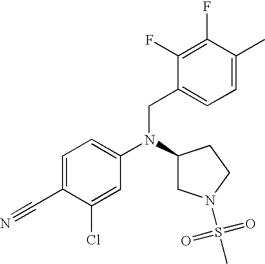

This compound was made according to general procedure of example 105 from 1-(bromomethyl)-2,3-difluoro-4-methylbenzene. LC-MS (ES) m/e 440.4 [M+H]⁺.

### Example 297

### 2-chloro-4-{[(2,3-difluorophenyl)methyl][(3S)-1-(methylsulfonyl)-3-pyrrolidinyl]amino}benzonitrile

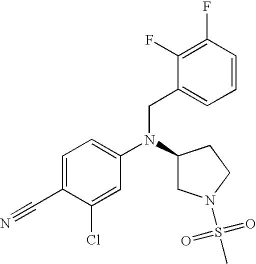

This compound was made according to general procedure of example 105 from 1-(bromomethyl)-2,3-difluorobenzene. LC-MS (ES) m/e 426.2 [M+H]⁺.

### Example 298

### 2-chloro-4-{[(3-chloro-2-fluorophenyl)methyl][(3S)-1-(methylsulfonyl)-3-pyrrolidinyl]amino}benzonitrile

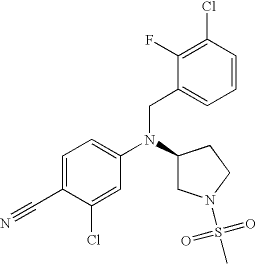

This compound was made according to general procedure of example 105 from 1-(bromomethyl)-3-chloro-2-fluorobenzene. LC-MS (ES) m/e 442.0 [M+H]⁺.

### Example 299

### 2-chloro-4-{{([3-chloro-2-fluoro-5-(trifluoromethyl)phenyl]methyl}[(3S)-1-(methylsulfonyl)-3-pyrrolidinyl]amino}benzonitrile

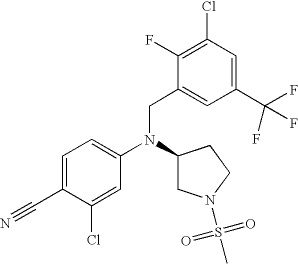

This compound was made according to general procedure of example 105 from 1-(bromomethyl)-3-chloro-2-fluoro-5-(trifluoromethyl)benzene. LC-MS (ES) m/e 510.4 [M+H]⁺.

### Example 300

### 2-chloro-4-{[(2,4-dichlorophenyl)methyl][(3S)-1-(methylsulfonyl)-3-pyrrolidinyl]amino}benzonitrile

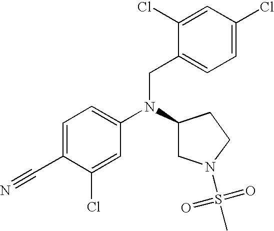

This compound was made according to general procedure of example 105 from 1-(bromomethyl)-2,4-dichlorobenzene. LC-MS (ES) m/e 458.0 [M+H]⁺.

### Example 301

### 2-chloro-4-{[(4-chloro-2-methylphenyl)methyl][(3S)-1-(methylsulfonyl)-3-pyrrolidinyl]amino}benzonitrile

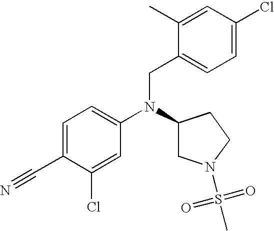

### a) 2-chloro-4-{[(4-chloro-2-methylphenyl)methyl][(3S)-3-pyrrolidinyl]amino}benzonitrile

This compound was made according to general procedures of example 1 (part b and c) from 1-(bromomethyl)-4-chloro-2-methylbenzene and used directly in the next step.

### b) 2-chloro-4-{[(4-chloro-2-methylphenyl)methyl][(3S)-1-(methylsulfonyl)-3-pyrrolidinyl]amino}benzonitrile

This compound was made according to general procedures of example 77 from 2-chloro-4-{[(4-chloro-2-methylphenyl)methyl][(3S)-3-pyrrolidinyl]amino}benzonitrile. LC-MS (ES) m/e 438.4 [M+H]⁺.

### Example 302

### 2-chloro-4-{[(3S)-1-(methylsulfonyl)-3-pyrrolidinyl][(2,3,4-trifluorophenyl)methyl]amino}benzonitrile

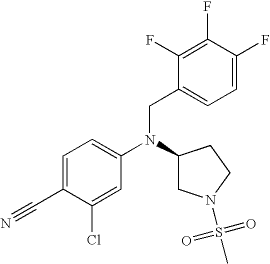

### a) 2-chloro-4-{(3S)-3-pyrrolidinyl[(2,3,4-trifluorophenyl)methyl]amino}benzonitrile

This compound was made according to general procedures of example 1 (part b and c) from 1-(bromomethyl)-2,3,4-trifluorobenzene and used directly in the next step.

### b) 2-chloro-4-{[(3S)-1-(methylsulfonyl)-3-pyrrolidinyl][(2,3,4-trifluorophenyl)methyl]amino}benzonitrile

This compound was made according to general procedures of example 77 from 2-chloro-4-{(3S)-3-pyrrolidinyl[(2,3,4-trifluorophenyl)methyl]amino}benzonitrile. LC-MS (ES) m/e 444.4 [M+H]⁺.

### Example 303

### 2-chloro-4-{[(3S)-1-(methylsulfonyl)-3-pyrrolidinyl][(2,4,6-trichlorophenyl)methyl]amino}benzonitrile

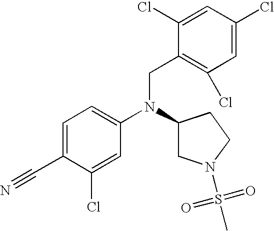

### a) 2-chloro-4-{(3S)-3-pyrrolidinyl[(2,4,6-trichlorophenyl)methyl]amino}benzonitrile

This compound was made according to general procedures of example 1 (part b and c) from 2-(bromomethyl)-1,3,5-trichlorobenzene and used directly in the next step.

### b) 2-chloro-4-{[(3S)-1-(methylsulfonyl)-3-pyrrolidinyl][(2,4,6-trichlorophenyl)methyl]amino}benzonitrile

This compound was made according to general procedures of example 77 from 2-chloro-4-{(3S)-3-pyrrolidinyl[(2,4,6-trichlorophenyl)methyl]amino}benzonitrile. LC-MS (ES) m/e 492.2 [M+H]⁺.

### Example 304

### 2-chloro-4-{[(3S)-1-(methylsulfonyl)-3-pyrrolidinyl][(2,3,5-trifluorophenyl)methyl]amino}benzonitrile

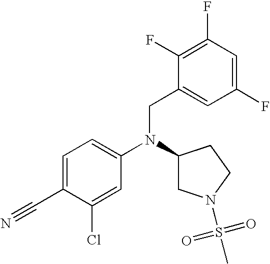

### a) 2-chloro-4-{(3S)-3-pyrrolidinyl[(2,3,5-trifluorophenyl)methyl]amino}benzonitrile

This compound was made according to general procedures of example 1 (part b and c) from 1-(bromomethyl)-2,3,5-trifluorobenzene and used directly in the next step.

### b) 2-chloro-4-{[(3S)-1-(methylsulfonyl)-3-pyrrolidinyl][(2,3,5-trifluorophenyl)methyl]amino}benzonitrile

This compound was made according to general procedures of example 77 from 2-chloro-4-{(3S)-3-pyrrolidinyl[(2,3,5-trifluorophenyl)methyl]amino}benzonitrile. LC-MS (ES) m/e 444.2 [M+H]⁺.

### Example 305

### 2-chloro-4-([(2-chloro-5-fluorophenyl)methyl]{(3S)-1-[(phenylmethyl)sulfonyl]-3-pyrrolidinyl}amino)benzonitrile

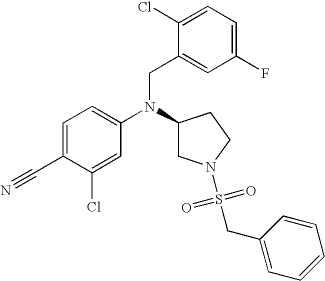

2-chloro-4-{[(2-chloro-5-fluorophenyl)methyl][(3S)-3-pyrrolidinyl]amino}benzonitrile (0.081 g, 0.22 mmol), phenylmethanesulfonyl chloride (0.106 g, 0.56 mmol), DIEA (0.086 g, 0.67 mmol) in CH₂Cl₂ (3 mL) were stirred at RT for 18 h. The reaction was concentrated and purified via column chromatography (eluted with 0% EtOAc in hexane grading to 80% EtOAc in hexane) to give the product (0.048 g, 42%) as a white powder. LC-MS (ES) m/e 518.3 [M+H]⁺.

### Example 306

### 2-chloro-4-{(cyclohexylmethyl)[(3S)-1-(propylsulfonyl)-3-pyrrolidinyl]amino}benzonitrile

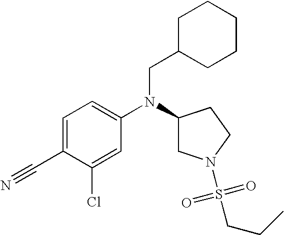

### a) 2-chloro-4-{(cyclohexylmethyl)[(3S)-3-pyrrolidinyl]amino}benzonitrile

This compound was made according to general procedures of example 1 (part b and c) from (bromomethyl)cyclohexane. LC-MS (ES) m/e 318.4 [M+H]⁺.

### b) 2-chloro-4-{(cyclohexylmethyl)[(3S)-1-(propylsulfonyl)-3-pyrrolidinyl]amino}benzonitrile

This compound was made according to general procedures of example 305 from 2-chloro-4-{(cyclohexylmethyl)[(3S)-3-pyrrolidinyl]amino}benzonitrile and 1-propanesulfonyl chloride. LC-MS (ES) m/e 424.3 [M+H]⁺.

### Example 307

### 2-chloro-4-((cyclohexylmethyl){(3S)-1-[(1-methylethy)sulfonyl]-3-pyrrolidinyl}amino)benzonitrile

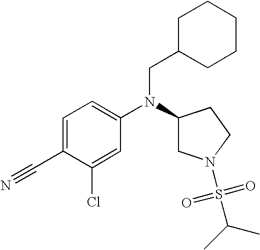

This compound was made according to general procedures of example 306 from 2-propanesulfonyl chloride. LC-MS (ES) m/e 424.3 [M+H]⁺.

### Example 308

### 2-chloro-4-{(cyclohexylmethyl)[(3S)-1-(ethylsulfonyl)-3-pyrrolidinyl]amino}benzonitrile

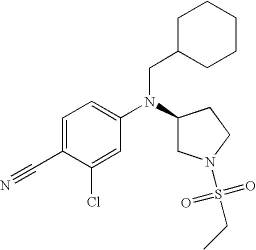

This compound was made according to general procedures of example 306 from ethanesulfonyl chloride. LC-MS (ES) m/e 390.4 [M+H]⁺.

### Example 309

### 2-chloro-4-((cyclobutylmethyl){(3S)-1-[cyclohexylmethyl)sulfonyl]-3-pyrrolidinyl}amino)benzonitrile

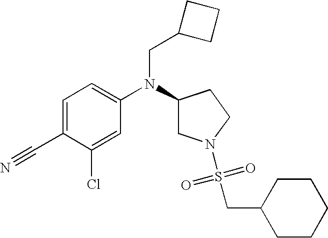

### a) 2-chloro-4-{(cyclobutylmethyl)[(3S)-3-pyrrolidinyl]amino}benzonitrile

This compound was made according to general procedures of example 1 (part b and c) from (bromomethyl)cyclobutane. LC-MS (ES) m/e 290.0 [M+H]⁺.

### b) 2-chloro-4-((cyclobutylmethyl){(3S)-1-[(cyclohexylmethyl)sulfonyl]-3-pyrrolidinyl}amino)benzonitrile

A solution of 2-chloro-4-{(cyclobutylmethyl)[(3S)-3-pyrrolidinyl]amino}benzonitrile (0.039 g, 0.135 mmol) and DIEA (0.052 g, 0.405 mmol) in DMF (1 mL) was added to cyclohexylmethanesulfonyl chloride (0.053 g, 0.27 mmol) and the reaction was stirred at RT for 18 h. MeOH (1 mL) was added and the reaction was diluted with H₂O, and extracted with EtOAc (2×). The organic extracts were washed with brine, dried over Na₂SO₄, and concentrated. The residue was purified by HPLC (40 mL/min, A: acetonitrile, B: water, A: 15 to 100% over 25 min) to give the product (0.033 g, 54%) as a brown solid. LC-MS (ES) m/e 450.3 [M+H]⁺.

### Example 310

### 2-chloro-4-((cyclobutylmethyl) {(3S)-1-[(3,3,3-trifluoropropyl)sulfonyl]-3-pyrrolidinyl}amino)benzonitrile

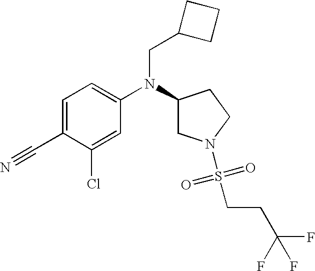

This compound was made according to general procedure of example 309 from 3,3,3-trifluoro-1-propanesulfonyl chloride. LC-MS (ES) m/e 450.4 [M+H]⁺.

### Example 311

### 2-chloro-4-{{(3S)-1-[(chloromethyl)sulfonyl]-3-pyrrolidinyl}[(2-fluorophenyl)methyl]amino}benzonitrile

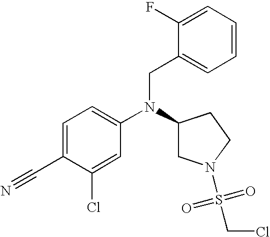

This compound was made according to general procedure of example 309 (part b) from 2-chloro-4-{[(2-fluorophenyl)methyl][(3S)-3-pyrrolidinyl]amino}benzonitrile and chloromethanesulfonyl chloride. LC-MS (ES) m/e 442.4 [M+H]⁺.

### Example 312

### 2-chloro-4-([(2-fluorophenyl)methyl]{(3S)-1-[(2,2,2-trifluoroethyl)sulfonyl]-3-pyrrolidinyl}amino)benzonitrile

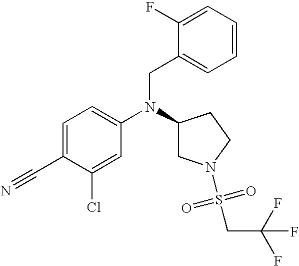

This compound was made according to general procedure of example 309 (part b) from 2-chloro-4-{[(2-fluorophenyl)methyl][(3S)-3-pyrrolidinyl]amino}benzonitrile and 2,2,2-trifluoroethanesulfonyl chloride except: after adding MeOH, the reaction was filtered and purified.

LC-MS (ES) m/e 476.1 [M+H]⁺.

### Example 313

### 2-chloro-4-([(2-fluorophenyl)methyl]{(3S)-1-[(phenylmethyl)sulfonyl]-3-pyrrolidinyl}amino)benzonitrile

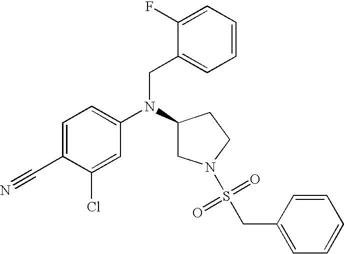

This compound was made according to general procedure of example 312 from phenylmethanesulfonyl chloride. LC-MS (ES) m/e 484.1 [M+H]⁺.

### Example 314

### 2-chloro-4-{[(3S)-1-(cyclopropylsulfonyl)-3-pyrrolidinyl][(2-fluorophenyl)methyl]amino}benzonitrile

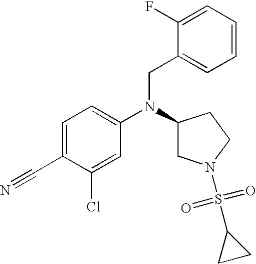

This compound was made according to general procedure of example 312 from cyclopropanesulfonyl chloride. LC-MS (ES) m/e 434.0 [M+H]⁺.

### Example 315

### 2-chloro-4-([(2-fluorophenyl)methyl]{(3S)-1-[(3,3,3-trifluoropropyl)sulfonyl]-3-pyrrolidinyl}amino)benzonitrile

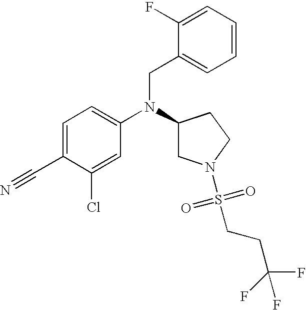

This compound was made according to general procedure of example 312 from 3,3,3-trifluoro-1-propanesulfonyl chloride. LC-MS (ES) m/e 490.1 [M+H]⁺.

### Example 316

### 4-{[(3S)-1-(butylsulfonyl)-3-pyrrolidinyl][(2-fluorophenyl)methyl]amino}-2-chlorobenzonitrile

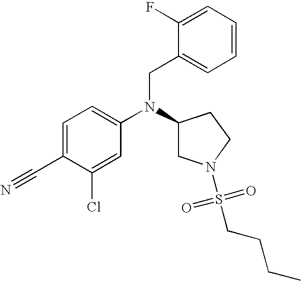

This compound was made according to general procedure of example 312 from 1-butanesulfonyl chloride. LC-MS (ES) m/e 450.1 [M+H]⁺.

### Example 317

### 2-chloro-4-{[(2-fluorophenyl)methyl][(3S)-1-(propylsulfonyl)-3-pyrrolidinyl]amino}benzonitrile

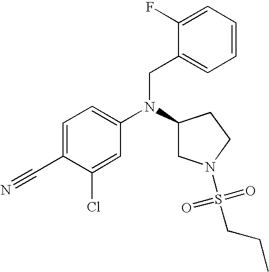

This compound was made according to general procedure of example 312 from 1-propanesulfonyl chloride. LC-MS (ES) m/e 436.1 [M+H]⁺.

### Example 318

### 2-chloro-4-((cyclohexylmethyl){(3S)-1-[(phenylmethyl)sulfonyl]-3-pyrrolidinyl}amino)benzonitrile

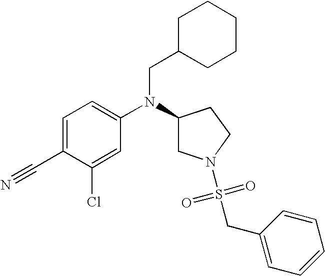

This compound was made according to general procedure of example 312 from 2-chloro-4-{(cyclohexylmethyl)[(3S)-3-pyrrolidinyl]amino}benzonitrile and phenylmethanesulfonyl chloride.

LC-MS (ES) m/e 472.1 [M+H]⁺.

### Example 319

### 2-chloro-4-{(cyclohexylmethyl)[(3S)-1-(cyclopropylsulfonyl)-3-pyrrolidinyl]amino}benzonitrile

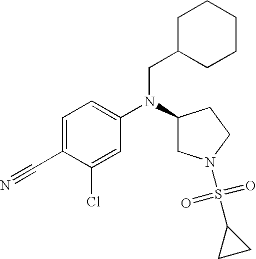

This compound was made according to general procedure of example 312 from 2-chloro-4-{(cyclohexylmethyl)[(3S)-3-pyrrolidinyl]amino}benzonitrile and cyclopropanesulfonyl chloride.

LC-MS (ES) m/e 422.1 [M+H]⁺.

### Example 320

### 2-chloro-4-((cyclohexylmethyl){(3S)-1-[(2,2,2-trifluoroethyl)sulfonyl]-3-pyrrolidinyl}amino)benzonitrile

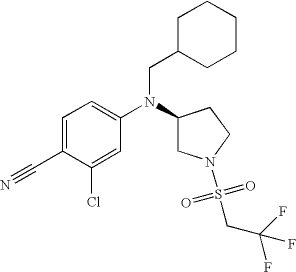

This compound was made according to general procedure of example 312 from 2-chloro-4-{(cyclohexylmethyl)[(3S)-3-pyrrolidinyl]amino}benzonitrile and 2,2,2-trifluoroethanesulfonyl chloride. LC-MS (ES) m/e 464.1 [M+H]⁺.

### Example 321

### 2-chloro-4-((cyclohexylmethyl) {(3S)-1-[(3,3,3-trifluoropropyl)sulfonyl]-3-pyrrolidinyl}amino)benzonitrile

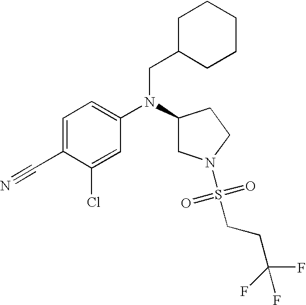

This compound was made according to general procedure of example 312 from 2-chloro-4-{(cyclohexylmethyl)[(3S)-3-pyrrolidinyl]amino}benzonitrile and 3,3,3-trifluoro-1-propanesulfonyl chloride. LC-MS (ES) m/c 478.1 [M+H]⁺.

### Example 322

### 4-[[(3S)-1-(butylsulfonyl)-3-pyrrolidinyl](cyclohexylmethyl)amino]-2-chlorobenzonitrile

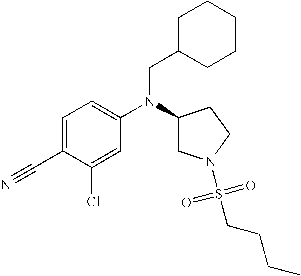

This compound was made according to general procedure of example 312 from 2-chloro-4-{(cyclohexylmethyl)[(3S)-3-pyrrolidinyl]amino}benzonitrile and 1-butanesulfonyl chloride. LC-MS (ES) m/e 438.1 [M+H]⁺.

### Example 323

### 2-chloro-4-({(3S)-1-[(phenylmethyl)sulfonyl]-3-pyrrolidinyl{}[2-(trifluoromethyl)phenyl]methyl}amino)benzonitrile

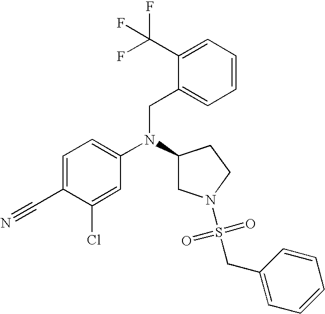

This compound was made according to general procedure of example 312 from 2-chloro-4-((3S)-3-pyrrolidinyl{[2-(trifluoromethyl)phenyl]methyl}amino)benzonitrile and phenylmethanesulfonyl chloride. LC-MS (ES) m/e 534.1 [M+H]⁺.

### Example 324

### 2-chloro-4-({[2-(trifluoromethyl)phenyl]methyl}{(3S)-1-[(3,3,3-trifluoropropyl)sulfonyl]-3-pyrrolidinyl}amino)benzonitrile

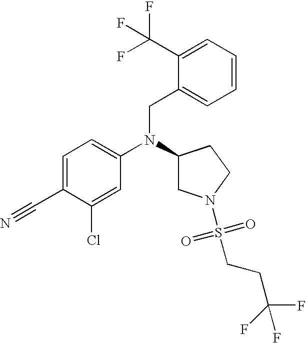

This compound was made according to general procedure of example 312 from 2-chloro-4-((3S)-3-pyrrolidinyl{[2-(trifluoromethyl)phenyl]methyl}amino)benzonitrile and 3,3,3-trifluoro-1-propanesulfonyl chloride. LC-MS (ES) m/e 540.1 [M+H]⁺.

### Example 325

### 2-chloro-4-({(3S)-1-[(2,2,2-trifluoroethyl)sulfonyl]-3-pyrrolidinyl}{[2-(trifluoromethyl)phenyl]methyl}amino)benzonitrile

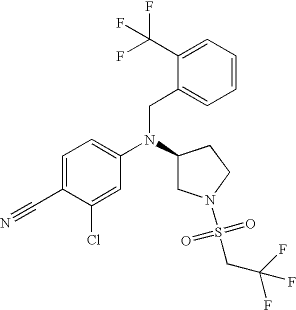

This compound was made according to general procedure of example 312 from 2-chloro-4-((3S)-3-pyrrolidinyl{[2-(trifluoromethyl)phenyl]methyl}amino)benzonitrile and 2,2,2-trifluoroethanesulfonyl chloride. LC-MS (ES) m/e 526.1 [M+H]⁺.

### Example 326

### 2-chloro-4-([(3S)-1-(cyclopropylsulfonyl)-3-pyrrolidinyl]{[2-(trifluoromethyl)phenyl]methyl}amino)benzonitrile

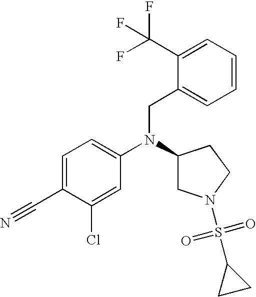

This compound was made according to general procedure of example 312 from 2-chloro-4-((3S)-3-pyrrolidinyl{[2-(trifluoromethyl)phenyl]methyl}amino)benzonitrile and cyclopropanesulfonyl chloride. LC-MS (ES) m/e 484.1 [M+H]⁺.

### Example 327

### 2-chloro-4-([(3S)-1-(propylsulfonyl)-3-pyrrolidinyl]{[2-(trifluoromethyl)phenyl]methyl}amino)benzonitrile

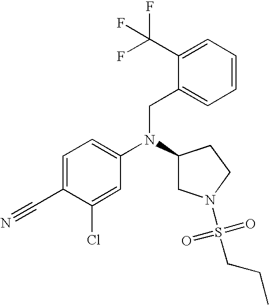

This compound was made according to general procedure of example 312 from 2-chloro-4-((3S)-3-pyrrolidinyl{[2-(trifluoromethyl)phenyl]methyl}amino)benzonitrile and 1-propanesulfonyl chloride. LC-MS (ES) m/e 484.1 [M+H]⁺.

### Example 328

### 4-([3S)-1-(butylsulfonyl)-3-pyrrolidinyl]{[2-(trifluoromethyl)phenyl]methyl}amino)-2-chlorobenzonitrile

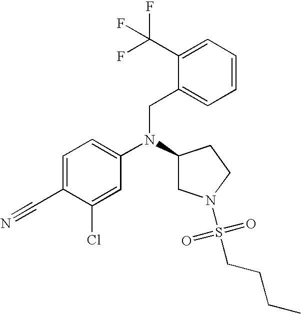

This compound was made according to general procedure of example 312 from 2-chloro-4-((3S)-3-pyrrolidinyl{[2-(trifluoromethyl)phenyl]methyl}amino)benzonitrile and 1-butanesulfonyl chloride.

LC-MS (ES) m/e 500.1 [M+H]⁺.

### Example 329

### 4-[[(3S)-1-(butylsulfonyl)-3-pyrrolidinyl](phenylmethyl)amino]-2-chlorobenzonitrile

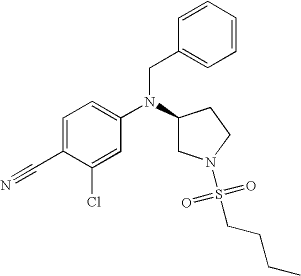

This compound was made according to general procedure of example 312 from 2-chloro-4-{(phenylmethyl)[(3S)-3-pyrrolidinyl]amino}benzonitrile and 1-butanesulfonyl chloride. LC-MS (ES) m/e 432.1 [M+H]⁺.

### Example 330

### 2-chloro-4-[[(3S)-1-(ethylsulfonyl)-3-pyrrolidinyl](phenylmethyl)amino]benzonitrile

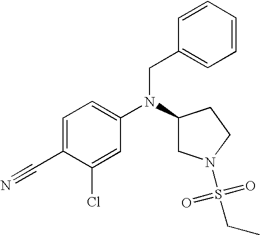

This compound was made according to general procedure of example 312 from 2-chloro-4-{(phenylmethyl)[(3S)-3-pyrrolidinyl]amino}benzonitrile and ethanesulfonyl chloride. LC-MS (ES) m/e 404.1 [M+H]⁺.

### Example 331

### 2-chloro-4-[[(3S)-1-(cyclopropylsulfonyl)-3-pyrrolidinyl](phenylmethyl)amino]benzonitrile

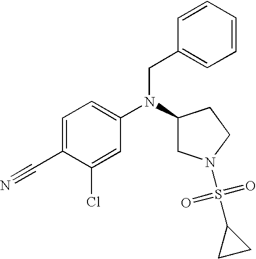

This compound was made according to general procedure of example 312 from 2-chloro-4-{(phenylmethyl)[(3S)-3-pyrrolidinyl]amino}benzonitrile and cyclopropanesulfonyl chloride. LC-MS (ES) m/e 416.1 [M+H]⁺.

### Example 332

### 2-chloro-4-((phenylmethyl){(3S)-1-[(3,3,3-trifluoropropyl)sulfonyl]-3-pyrrolidinyl}amino)benzonitrile

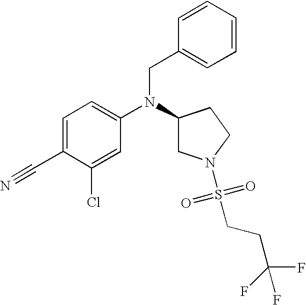

This compound was made according to general procedure of example 312 from 2-chloro-4-{(phenylmethyl)[(3S)-3-pyrrolidinyl]amino}benzonitrile and 3,3,3-trifluoro-1-propanesulfonyl chloride. LC-MS (ES) m/e 472.0 [M+H]⁺.

### Example 333

### 2-chloro-4-((phenylmethyl) {(3S)-1-[(phenylmethyl)sulfonyl]-3-pyrrolidinyl}amino)benzonitrile

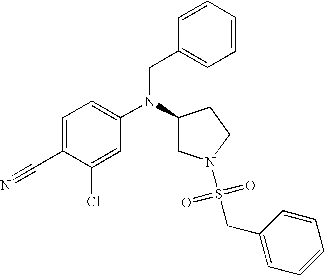

This compound was made according to general procedure of example 312 from 2-chloro-4-{(phenylmethyl)[(3S)-3-pyrrolidinyl]amino}benzonitrile and phenylmethanesulfonyl chloride. LC-MS (ES) m/c 466.1 [M+H]⁺.

### Example 334

### 2-chloro-4-[{(3S)-1-[1-methylethyl)sulfonyl]-3-pyrrolidinyl}(phenylmethyl)amino]benzonitrile

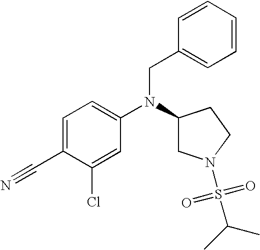

This compound was made according to general procedure of example 312 from 2-chloro-4-{(phenylmethyl)[(3S)-3-pyrrolidinyl]amino}benzonitrile and 2-propanesulfonyl chloride. LC-MS (ES) m/e 418.1 [M+H]⁺.

### Example 335

### 2-chloro-4-((phenylmethyl){(3S)-1-[(2,2,2-trifluoroethyl)sulfonyl]-3-pyrrolidinyl}amino)benzonitrile

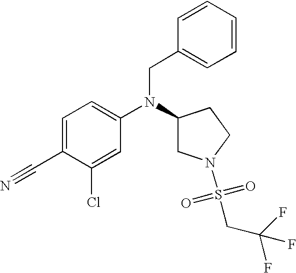

This compound was made according to general procedure of example 312 from 2-chloro-4-{(phenylmethyl)[(3S)-3-pyrrolidinyl]amino}benzonitrile and 2,2,2-trifluoroethanesulfonyl chloride.

LC-MS (ES) m/e 458.0 [M+H]⁺.

### Example 336

### 2-chloro-4-{(phenylmethyl)[(3S)-1-(propylsulfonyl)-3-pyrrolidinyl]amino}benzonitrile

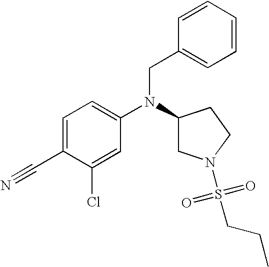

This compound was made according to general procedure of example 312 from 2-chloro-4-{(phenylmethyl)[(3S)-3-pyrrolidinyl]amino}benzonitrile and 1-propanesulfonyl chloride. LC-MS (ES) m/e 418.1 [M+H]⁺.

### Example 337

### 2-chloro-4-{[(3S)-1-(cyclopropylsulfonyl)-3-pyrrolidinyl[](2,5-dichlorophenyl)methyl]amino}benzonitrile

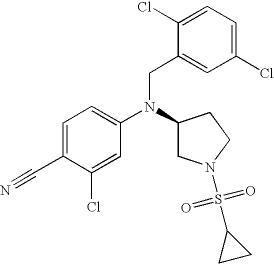

This compound was made according to general procedure of example 312 from 2-chloro-4-{[(2,5-dichlorophenyl)methyl][(3S)-3-pyrrolidinyl]amino}benzonitrile and cyclopropanesulfonyl chloride.

LC-MS (ES) m/e 486.0 [M+H]⁺.

### Example 338

### 2-chloro-4-{[(2,5-dichlorophenyl)methyl][(3S)-1-(propylsulfonyl)-3-pyrrolidinyl]amino}benzonitrile

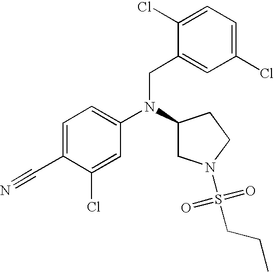

This compound was made according to general procedure of example 312 from 2-chloro-4-{[(2,5-dichlorophenyl)methyl][(3S)-3-pyrrolidinyl]amino}benzonitrile and 1-propanesulfonyl chloride.

LC-MS (ES) m/e 486.0 [M+H]⁺.

### Example 339

### 2-chloro-4-([(2,5-dichlorophenyl)methyl]{(3S)-1-[(phenylmethyl)sulfonyl]-3-pyrrolidinyl}amino)benzonitrile

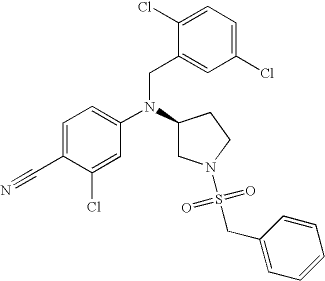

This compound was made according to general procedure of example 312 from 2-chloro-4-{[(2,5-dichlorophenyl)methyl][(3S)-3-pyrrolidinyl]amino}benzonitrile and phenylmethanesulfonyl chloride. LC-MS (ES) m/e 534.1 [M+H]⁺.

### Example 340

### 4-{[(3S)-1-(butylsulfonyl)-3-pyrrolidinyl][(2,5-dichlorophenyl)methyl]-amino}-2-chlorobenzonitrile

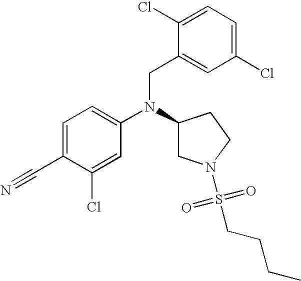

This compound was made according to general procedure of example 312 from 2-chloro-4-{[(2,5-dichlorophenyl)methyl][(3S)-3-pyrrolidinyl]amino}benzonitrile and 1-butanesulfonyl chloride.

LC-MS (ES) m/e 500.0 [M+H]⁺.

### Example 341

### 2-chloro-4-([(2,5-dichlorophenyl)methyl]{(3S)-1-[(2,2,2-trifluoroethyl)sulfonyl]-3-pyrrolidinyl}amino)benzonitrile

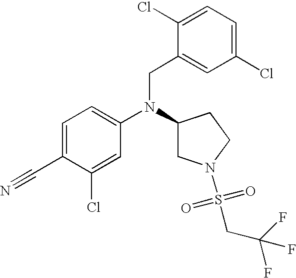

This compound was made according to general procedure of example 312 from 2-chloro-4-{[(2,5-dichlorophenyl)methyl][(3S)-3-pyrrolidinyl]amino}benzonitrile and 2,2,2-trifluoroethanesulfonyl chloride. LC-MS (ES) m/e 526.0 [M+H]⁺.

### Example 342

### 2-chloro-4-([(2,5-dichlorophenyl)methyl]{(3S)1-[(3,3,3-trifluoropropyl)sulfonyl]-3-pyrrolidinyl}amino)benzonitrile

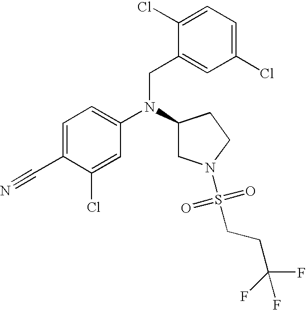

This compound was made according to general procedure of example 312 from 2-chloro-4-{[(2,5-dichlorophenyl)methyl][(3S)-3-pyrrolidinyl]amino}benzonitrile and 3,3,3-trifluoro-1-propanesulfonyl chloride. LC-MS (ES) m/e 540.0 [M+H]⁺.

### Example 343

### 2-chloro-4-{[(3S)-1-(cyclopropylsulfonyl)-3-pyrrolidinyl][(2,5-difluorophenyl)methyl]amino}benzonitrile

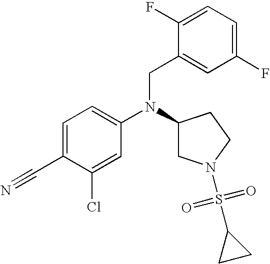

This compound was made according to general procedure of example 312 from 2-chloro-4-{[(2,5-difluorophenyl)methyl][(3S)-3-pyrrolidinyl]amino}benzonitrile and cyclopropanesulfonyl chloride.

LC-MS (ES) m/e 452.0 [M+H]⁺.

### Example 344

### 4-{[(3S)-1-(butylsulfonyl)-3-pyrrolidinyl][(2,5-difluorophenyl)methyl]-amino}-2-chlorobenzonitrile

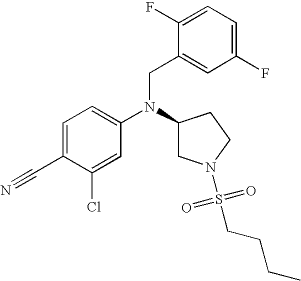

This compound was made according to general procedure of example 312 from 2-chloro-4-{[(2,5-difluorophenyl)methyl][(3S)-3-pyrrolidinyl]amino}benzonitrile and 1-butanesulfonyl chloride.

LC-MS (ES) m/e 468.1 [M+H]⁺.

### Example 345

### 2-chloro-4-{[(2,5-difluorophenyl)methyl][(3S)-1-(propylsulfonyl)-3-pyrrolidinyl]amino}benzonitrile

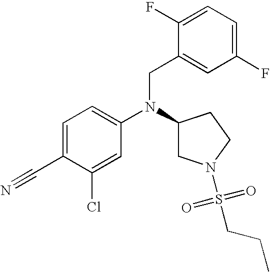

This compound was made according to general procedure of example 312 from 2-chloro-4-{[(2,5-difluorophenyl)methyl][(3S)-3-pyrrolidinyl]amino}benzonitrile and 1-propanesulfonyl chloride.

LC-MS (ES) m/e 454.0 [M+H]⁺.

### Example 346

### 2-chloro-4-([(5-fluoro-2-methylphenyl)methyl]{(3S)-1-[(2,2,2-trifluoroethyl)sulfonyl]-3-pyrrolidinyl}amino)benzonitrile

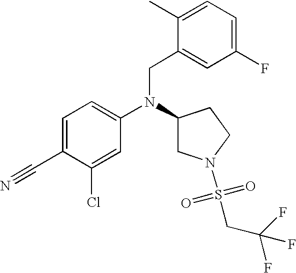

This compound was made according to general procedure of example 312 from 2-chloro-4-{[(5-fluoro-2-methylphenyl)methyl][(3S)-3-pyrrolidinyl]amino}benzonitrile and 2,2,2-trifluoroethanesulfonyl chloride. LC-MS (ES) m/e 490.1 [M+H]⁺.

### Example 347

### 2-chloro-4-([(5-fluoro-2-methylphenyl)methyl]{(3S)-1-[(3,3,3-trifluoropropyl)sulfonyl]-3-pyrrolidinyl}amino)benzonitrile

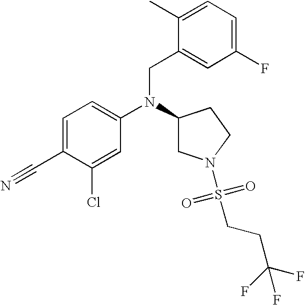

This compound was made according to general procedure of example 312 from 2-chloro-4-{[(5-fluoro-2-methylphenyl)methyl][(3S)-3-pyrrolidinyl]amino}benzonitrile and 3,3,3-trifluoro-1-propanesulfonyl chloride. LC-MS (ES) m/e 504.1 [M+H]⁺.

### Example 348

### 2-chloro-4-{[(3S)-1-(cyclopropylsulfonyl)-3-pyrrolidinyl][(5-fluoro-2-methylphenyl)methyl]amino}benzonitrile

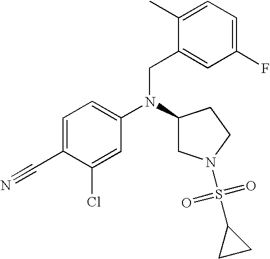

This compound was made according to general procedure of example 312 from 2-chloro-4-{[(5-fluoro-2-methylphenyl)methyl][(3S)-3-pyrrolidinyl]amino}benzonitrile and cyclopropanesulfonyl chloride. LC-MS (ES) m/e 448.1 [M+H]⁺.

### Example 349

### 2-chloro-4-{[(5-fluoro-2-methylphenyl)methyl][(3S)-1-(propylsulfonyl)-3-pyrrolidinyl]amino}benzonitrile

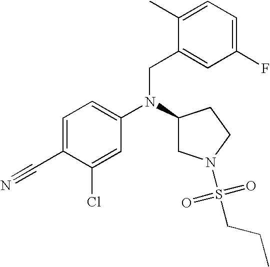

This compound was made according to general procedure of example 312 from 2-chloro-4-{[(5-fluoro-2-methylphenyl)methyl][(3S)-3-pyrrolidinyl]amino}benzonitrile and 1-propanesulfonyl chloride. LC-MS (ES) m/e 450.1 [M+H]⁺.

### Example 350

### 2-chloro-4-([(5-fluoro-2-methylphenyl)methyl]{(3S)-1-[(phenylmethyl)sulfonyl]-3-pyrrolidinyl}amino)benzonitrile

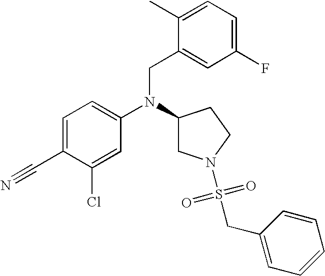

This compound was made according to general procedure of example 312 from 2-chloro-4-{[(5-fluoro-2-methylphenyl)methyl][(3S)-3-pyrrolidinyl]amino}benzonitrile and phenylmethanesulfonyl chloride. LC-MS (ES) m/e 498.1 [M+H]⁺.

### Example 351

### 2-chloro-4-{[(2-methylphenyl)methyl][(3S)-1-(2-pyridinylsulfonyl)-3-pyrrolidinyl]amino}benzonitrile

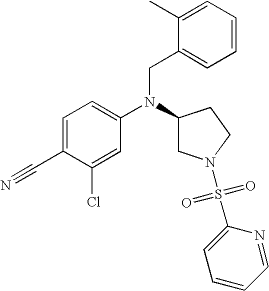

This compound was made according to general procedure of example 77 from 2-pyridinesulfonyl chloride. LC-MS (ES) m/e 467.4 [M+H]⁺.

### Example 352

### 2-chloro-4-([(3S)-1-(2-pyridinylsulfonyl)-3-pyrrolidinyl]{[2-(trifluoromethyl)phenyl]methyl}amino)benzonitrile

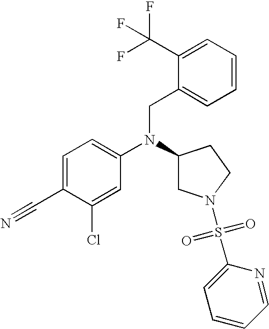

This compound was made according to general procedure of example 77 from 2-chloro-4-((3S)-3-pyrrolidinyl{[2-(trifluoromethyl)phenyl]methyl}amino)benzonitrile and 2-pyridinesulfonyl chloride. LC-MS (ES) m/e 521.4 [M+H]⁺.

### Example 353

### 2-chloro-4-{(2-methylpropyl)[(3S)-1-(methylsulfonyl)-3-pyrrolidinyl]amino}benzonitrile

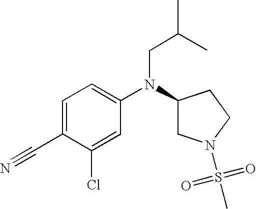

This compound was made according to procedure of example 121 from 2-chloro-4-{(2-methyl-2-propen-1-yl)[(3S)-1-(methylsulfonyl)-3-pyrrolidinyl]amino}benzonitrile. LC-MS (ES) m/e 356.4[M+H]⁺.

1. A compound represented by the following formula: 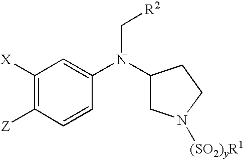
or a pharmaceutically acceptable salt thereof;
wherein Z is Cl, NO₂, OCH₃, or CN;
X is H, F, Cl, Br, or CF₃;
R¹ is H, C₁-C₆-alkyl, CF₃, C₁-C₆-alkoxycarbonyl-C₁-C₆-alkyl, C₃-C₆-cycloalkyl, heterocycloalkyl, C₁-C₆-alkyl-heterocycloalkyl, heteroaryl-(R³)ₙ, phenyl-(R^{3′})ₙ, or —CH₂R⁴;
y is 0 or 1, with the proviso that when R¹ is H, y is 0; and
R² is C₁-C₅-alkyl, C₁-C₆-alkoxycarbonyl, C₁-C₆-alkoxycarbonyl-C₁-C₅-alkyl, C₁-C₅-alkyloxy-C₁-C₅-alkyl, C₃-C₆-cycloalkyl, heterocycloalkyl, C₁-C₆-alkyl-heterocycloalkyl, C₂-C₄-alkenyl, naphthyl, heteroaryl-(R³)ₙ, or phenyl-(R^{3′})ₙ, where n is 0, 1, 2, or 3;
each R³ is independently CH₃, F, Cl, Br, CF₃, C₁-C₆-alkoxy, C₁-C₆-alkoxycarbonyl, dimethylamino, C₂-C₄-alkenyl, or CN, or where 2 of the R³ groups, together with the heteroaryl ring to which they are attached form a fused bicyclic ring;
each R^{3′} is independently C₁-C₆-alkyl, F, Cl, Br, CF₃, C₁-C₆-alkoxy, dimethylamino, amido, C₂-C₄-alkenyl, nitro, —COO—C₁-C₆-alkyl, OH, COOH, or CN, or where 2 of the R^{3′} groups, together with the phenyl ring to which they are attached form a fused bicyclic ring; and
R⁴ is F, Cl, Br, C₁-C₆-alkyloxy-C₁-C₆-alkyl, CF₃, CH₂CF₃, COOH, CH₂CN, CN, C₁-C₆-alkylcarbonyl, heterocycloalkyl, C₁-C₆-alkyl-heterocycloalkyl, heterocycloalkyl-CH₂—, aminocarbonyl, aminocarbonyl-CH₂—, di-C₁-C₆-alkylaminocarbonyl, C₂-C₄-alkenyl, hydroxy-C₁-C₆-alkyl, naphthyl, heteroaryl-(R³)ₙ, phenyl-(R^{3′})ₙ, or CH₂— phenyl-(R^{3′})ₙ.

2. The compound of claim 1 or a pharmaceutically acceptable salt thereof wherein X is Cl and Z is CN.

3. The compound of claim 2 or a pharmaceutically acceptable salt thereof which is represented by the following formula: 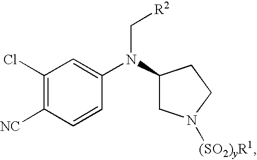
wherein:
R¹ is H, isopropropyl, C(CH₃)₂C(O)OCH₂CH₃, C₃-C₆-cycloalkyl, or CH₂R⁴, R⁴ is H, C₁-C₅-alkyl, 2,2,2-trifluoroethyl, methoxymethyl, CN, C₃-C₆-cycloalkyl, phenyl-(R^{3′})ₙ, pyridinyl, oxidopyridinyl, dihydrobenzodioxinyl, thienyl, methylthienyl, furanyl, methylfuranyl, imidizolyl, methylimidazolyl, isooxazolyl, methylisooxazolyl, piperidinyl, methylpiperidinyl, pyrazinyl, morpholino, 1,3-dioxolan-2-yl, CO₂CH₂CH₃; CO₂-t-butyl, CH₂CO₂-t-butyl, C(O)NH₂, C(O)N(CH₃)₂, C(O)CH₃, C₂-C₄-alkenyl, or hydroxy-C₁-C₆-alkyl; and
R² is C₁-C₅-alkyl, 2-propenyl, benzothiadiazolyl, isooxazolyl, methylisooxazolyl, phenylisooxazolyl, 1,3-benzodioxolyl, bromo-1,3-benzodioxolyl, piperidinyl, methylpiperidinyl, pyrazinyl, morpholino, 1,3-dioxolan-2-yl, furanyl, trifluoromethylfuranyl, naphthyl, thienyl, methylthienyl, methoxypyridinylthienyl, bromothienyl, furanyl, methylfuranyl, C₃-C₆-cycloalkyl, phenyl-(R^{3′})ₙ, dimethylamino, C₂-C₄-alkenyl, or CN; where each R^{3′} is independently CH₃, F, Cl, Br, CF₃, or OCH₃; and n is 0, 1, 2, or 3.

4. The compound of claim 3 or a pharmaceutically acceptable salt thereof which is represented by the following formula: 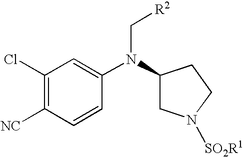
wherein
R¹ is CH₂R⁴ where R⁴ is H or C₁-C₅-alkyl; and
R² is phenyl-(R^{3′})ₙ, where each R^{3′} is independently CH₃, F, Cl, Br, or CF₃.

5. The compound of claim 1 or a pharmaceutically acceptable salt thereof wherein y=1.

6. The compound of claim 3 or a pharmaceutically acceptable salt thereof wherein y=0; and R¹ is C(CH₃)₂C(O)OCH₂CH₃, C₃-C₆-cycloalkyl, or CH₂R⁴, 2,2,2-trifluoroethyl, methoxymethyl, CN, C₃-C₆-cycloalkyl, phenyl-(R^{3′})ₙ, pyridinyl, oxidopyridinyl, dihydrobenzodioxinyl, thienyl, methylthienyl, furanyl, methylfuranyl, imidizolyl, methylimidazolyl, isooxazolyl, methylisooxazolyl, piperidinyl, methylpiperidinyl, pyrazinyl, morpholino, 1,3-dioxolan-2-yl, CO₂CH₂CH₃, CO₂-t-butyl, CH₂CO₂-t-butyl, C(O)NH₂, C(O)N(CH₃)₂, C(O)CH₃, C₂-C₄-alkenyl, or hydroxy-C₁-C₆-alkyl.

7. The compound of claim 4 or a pharmaceutically acceptable salt thereof which is represented by the following structure: 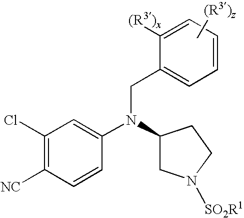
where x is 0 or 1 and z is 0, 1, or 2, with the proviso that when x is 0, z is 0.

8. The compound of claim 1 or a pharmaceutically acceptable salt thereof which has an IC₅₀ of less than 10 μM.

9. A method comprising administering to a patient in need thereof an effective amount of the compound of claim 3 or a pharmaceutically-acceptable salt thereof to treat endometreosis or uterine fibroids.

10. A method comprising administering to a patient in need thereof an effective amount of the compound of claim 2 or a pharmaceutically-acceptable salt thereof to treat endometreosis or uterine fibroids.

11. A composition comprising 1) the compound of claim 2 or a pharmaceutically-acceptable salt thereof; and 2) a pharmaceutically acceptable carrier therefor.

12. A compound selected from the group consisting of:
2-chloro-4-{[(2-methylphenyl)methyl][(3S)-1-(methylsulfonyl)-3-pyrrolidinyl]amino}benzonitrile;
2-chloro-4-{[(2-chlorophenyl)methyl][(3S)-1-(methylsulfonyl)-3-pyrrolidinyl]amino}benzonitrile;
2-chloro-4-{[(2-fluorophenyl)methyl][(3S)-1-(methylsulfonyl)-3-pyrrolidinyl]amino}benzonitrile;
2-chloro-4-([(3S)-1-methyl-3-pyrrolidinyl]{[2-(trifluoromethyl)phenyl]methyl}amino)benzonitrile;
2-chloro-4-{[(2-chloro-5-fluorophenyl)methyl][(3S)-1-(methylsulfonyl)-3-pyrrolidinyl]amino}benzonitrile;
2-chloro-4-{[(5-fluoro-2-methylphenyl)methyl][(3S)-1-(methylsulfonyl)-3-pyrrolidinyl]amino}benzonitrile;
2-chloro-4-{[(2,3-difluorophenyl)methyl][(3S)-1-(methylsulfonyl)-3-pyrrolidinyl]amino}benzonitrile;
2-chloro-4-{[(2,5-difluorophenyl)methyl][(3S)-1-(methylsulfonyl)-3-pyrrolidinyl]amino}benzonitrile;
2-chloro-4-{[(2,4-difluorophenyl)methyl][(3S)-1-(methylsulfonyl)-3-pyrrolidinyl]amino}benzonitrile; and
2-chloro-4-{[(2,5-dichlorophenyl)methyl][(3S)-1-methyl-3-pyrrolidinyl]amino}benzonitrile;
or a pharmaceutically acceptable salt thereof.

13. The compound of claim 12 which is 2-chloro-4-{[(2-chlorophenyl)methyl][(3S)-1-(methylsulfonyl)-3-pyrrolidinyl]amino}benzonitrile or a pharmaceutically acceptable salt thereof.

14. The compound of claim 12 which is 2-chloro-4-{[(2-methylphenyl)methyl][(3>S)-1-(methylsulfonyl)-3-pyrrolidinyl]amino}benzonitrile or a pharmaceutically acceptable salt thereof.

15. The compound of claim 12 which is 2-chloro-4-([(3S)-1-methyl-3-pyrrolidinyl]{[2-(trifluoromethyl)phenyl]methyl}amino)benzonitrile or a pharmaceutically acceptable salt thereof.

16. The compound of claim 12 which is 2-chloro-4-{[(2-fluorophenyl)methyl][(3S)-1-(methylsulfonyl)-3-pyrrolidinyl]amino}benzonitrile or a pharmaceutically acceptable salt thereof.

17. The compound of claim 12 which is 2-chloro-4-{[(2,3-difluorophenyl)methyl][(3S)-1-(methylsulfonyl)-3-pyrrolidinyl]amino}benzonitrile or a pharmaceutically acceptable salt thereof.

18. The compound of claim 12 which is 2-chloro-4-{[(2,5-difluorophenyl)methyl][(3S)-1-(methylsulfonyl)-3-pyrrolidinyl]amino}benzonitrile or a pharmaceutically acceptable salt thereof.

19. The compound of claim 12 which is 2-chloro-4-{[(2,4-difluorophenyl)methyl][(3S)-1-(methylsulfonyl)-3-pyrrolidinyl]amino}benzonitrile or a pharmaceutically acceptable salt thereof.

20. The compound of claim 12 which is 2-chloro-4-{[(2,5-dichlorophenyl)methyl][(3S)-1-methyl-3-pyrrolidinyl]amino}benzonitrile or a pharmaceutically acceptable salt thereof or a pharmaceutically acceptable salt thereof.
